SEQUENCE LISTING (105 sequences)

SEQ ID NO: 1 - protein, 326 aa, Bernardia pulchella
MEEEKMKKKEEGLRVINARDVYKTNMFHSLLSLMLWIGSIHFNFFLVFISFIFLPVPKFLFVVGLLLVLMFIPINPRSNFGLRLCRYMSRHACSYFPITLHVEDMNAFRSDRAYVFGYEPHSVFPLGVAILSDHMGFMPLPKIKVLASSTIFRTPFLRHIWTWCGLAPATRKNFTSLLASGYSCIVIPGGVQETFYMKHGSEIAFLKTRRGFVRLAIEMGKPLVPVFCFGQTNVYRWWRPGGKLVMKFARIIRFAPLFFWGVLGSPLPLPHPVHVVVGRPIELTQNPQPTMEEVAEVHSKFVAALRDLFERHKDRVGCGDIALEIH

SEQ ID NO: 2 - protein, 550 aa, Bernardia pulchella
MTILETPDTLISSSPTATAGVITSDLNLSLRRRRWTSSNSDSAIAELASKIDDKEENGGLIDEVKSKEERRENLSMNPIASTAAVTELETLTSNAKESVVNNNNDNDDNDSSNKFENSENHERGIDIKFTYRPSVPAHRSHKESPLSSDLIFKQSHAGLFNLCIVVLVAVNGRLIIENLMKYGWLIKTGFWFSSRSLRDWPLFMCCLSLPVFPLAAYLVEKMAYRKCISAPIVIFLHVIITSAAILYPVSVILSCESAVLSGVTLMLFACIVWLKLVSYAHTNYDMRAVADSVDKGHASNTLSAEYSHDVSFKSLVYFMVAPTLCYQTVYPRTASIRKGWVFRQFVKLIIFTGFMGFIIEQYINPIVQNSQHPLKGNLLYAIERVLKLSVPNLYVWLCMFYCFFHLWLNILAELLRFGDREFYKDWWNAKTVEEYWRMWNMPVHKWMVRHIYFPCLRHKIPKEVALVIAFFVSAVFHELCIAVPCHIFKLWAAFGIMFQIPLVLITKYLQNKFRSSMVGNMIFWFFFCILGQPMCVLLYYHDLMNRKGKN

SEQ ID NO: 3 - protein, 329 aa, Bernardia pulchella
MSVIGGSSKVLLRPRKANKVSSNCFFRDNGYLNYYYNYNEGVVRCGGDCSKSIKKKLKLVKSLTKDLSMISDMADFQLHQAQVTSLQDASRALMQQLEELKAKEKELERQKKEEKKTNRKPIETMMMDSESSTTSSSSSSESDCGDEEVIRMSRLNYDSIAVAEPVSTLTMLPNKQEQTCDCGDEEVIRMSRLKYDSIAVAEPVSTLTMLPNKQEQTCVISSNKMNKPNGLPVGELTEKKIEVCMGNKCKKSGSVALMDEFQRVMGSESVVCGCKCMGKCRDGPNVRVVDSAASSVSSLCIGVGLGDVGDIANRILGKEKVLGLAIASP

SEQ ID NO: 4 - protein, 154 aa, Bernardia pulchella
MENYIYQSVAYVILLSFFSSSNHAVALNIGVQTANSAITLSKECSRKCESEFCSVPPFLRYGKYCGLLYSGCPGEKPCDGLDACCMKHDACVQAKNNDYLSQECSQTFINCMNSFKKTGGHTFQGNQCQVDEVIEVISLVMEAALLAGRYLHKP

SEQ ID NO: 5 - protein, 511 aa, Euphorbia lagascae
MSILKRRSSKVRSSSDLSDFQNDDDDNKKEREKPKRRQTGRGRRGKKWTCLDSCCWFIGFICSMWWFLLFLFNAMPSSFPQYVTEAITGPSSDPPGVKLKKEGLRVKNPVVFVPGIVTGGLELWEGHQCADGLFRKRLWGGAFGEVYRRPLCWVEHMSLDNETGLDPPGVRVRPVSGLVAADYFAPGYFIWAVLIANLARLGYEEKTMYMAAYDWRLSFQNTEVRDQTLSRIKSNIELMVATNGGEKVVVIPHSMGALYFLHFMKWVEAPAPMGGGGGADWCAKHIKAVMNIGGPFLGVPKAVSGVFSNEARDIAAARAYAPVFLDKDVFGLQTLQHLMRMTRTWDSTMSMLPKGGDTIWGGLDWSPEGIYNCGAKKPKNNTTNSVGQTGKGTSSFKDGVNYGRIISFGKDVAELHSTKIDRKDFRDVFRGNKDTNSCDIWTEFHEMDIDALKAVTDYKVYTADSILDLLHFVAPKLMARGDVHFSHGIADNLDDPKYGHYKYWSNPLETK

SEQ ID NO: 6 - protein, 682 aa, Bernardia pulchella
MPLIRRKKPTKETIEDSTASKEEEKGKEQEEEKEEEDKNNKKKYPNKRNSQIKPKWSCVDNCCWFVGCICISWWVLLFLYNAMPASLPQYVTEAITGPLPDPPGVKLRKEGLKAKHPVVFVPGIVTAGLELWEGHECADGLFRKRLWGGTFGEVYKRPLCWVEHMSLDNETGLDPSGIRVRPVSGLVAADYFAPGYFVWAVLIANLARIGYEEKTMYMASYDWRLSFQNTEVRDQTLSRMKSNVELMVATNGGNKAVIVPHSMGVLYFLHFMKWVEAPAPMGGDGGPDWCARHIKAVMNIGGPFLGVPKAVAGLFSAEARDIAVARAIAPSFLDNDIFRLQTLQHIMRMSRTWDSTMSMIPRGGDTIWGDLDWSPEEGYVTRKRKQKKSTADYANQDGDESESSQRKCVKYGRMISFGKDVAEAPSSDIERIDFRGAVKGHSVVNSTCCDVWTEYHEMGYGGIKAVAEYKAYTAASIIDLLQFVAPKMMERGSAHFSYGIADNLDDPKYKHYKYWSNPLETTLPNAPEMEIFSMYGVGIPTERAYVYKLYPASECYIPFQIDTSAEGGDEDDCLRDGVYTVDGDETVPVLSTGFMSAKAWRVKTRFNPSGIQTYNREYNHSPPANLLEGRGTQSGAHVDIMGNFALIEDIMRVAAGATGEELGGDQVYSDIFKWSDKINLPL

SEQ ID NO: 7 - protein, 389 aa, Bernardia pulchella
MGYIGKHGEAALRRYKYSGEDHSYVAKYILQPFWTRFVRFFPLWMPPNMITLTGFMFLVVSALLGYVYSPHLDTPPPRWVHFAHGLLLFLYQTFDAVDGKQARRTNSSSPLGELFDHGCDALACAFESLAFSSTAMCGRDAFWFWVISAVPFYGATWEHFFTDTLILPAINGPTEGLMLIYVAHFFTATVGAEWWVQHFAKSFPFLSWVPFISEIQTYRAVLYLMTVFGVIPTAACNMSNVYKVVKARNSSMFLALAMLYPFAVLMGGMLLWDYLSPSDLMWDYPHLVVLGTGLAFGFLVGRMILAHLCEEPKGLKTNMCFSLLYLPVAIANALTARLNNGVPLVNEFWVLLGYCLFTGGLYLHFATSVIHEITTSLGINCFRITRKEA

SEQ ID NO: 8 - protein, 459 aa, Bernardia pulchella
MGLEMDSMASAIGVSVPVLRFLLCFVATIPVSCMHRVVPGRLGKHAYAALSGVLLSYLSFGFSSNLHFLVPMLLGYASMVLFRSHCGILAFILGFGYLIGCHAYYMSGDAWKEGGIDATGALMVLTLKVISCAINYNDGLLKEEELRESQKKNRLIKLPSLIEYIGYCLCCGSHFAGPVYEVKDYLEWTEKKGIWTHTPSPYGATVRAILQAGICMAIYLYLVPHYPLSRFNDPLYQEWGFLKRLSYQYMSGFTARWKYYFIWSISEASMIISTLGFSGWTGSSPPKPRWDRAKNVDILGVEFAKSAAELPLMWNIQVSTWLRHYVYDRLVPKGKKAGFVQLLATQTTSAVWHGLYPGYIIFFIQSALMIEGSKVIYRWQQAIPPKKVLFKKMLVFINMAYTLLVLNCSCVGFMVLSFHETIAAYGSVYYVGTIVPIVIFLLGFIIKPARSVKSKGRKD

SEQ ID NO: 9 - protein, 466 aa, Bernardia pulchella
MDLHMESMASSIGVSIPVLRFLLCFVATIPVSFVHRVIPGRLGKHVYAALSGAVLSYLSFGFSSNLHFLVPMLLGYGSMVLFRSHCGIMTFALGFGYLIGCHVHYMSGDAWKEGGIDATGALMVLTLKVISCAMNYNDGLLKEEDLRESQKKNRLIKLPSLIEYFGYCLCCASHFAGPVYEMKDYLDWTERKGIWARTEKGPSPSPYWATVRAVMQAAICMGIYLYLVPYYPLSRFTDSVYQEWGFWKRLSYQYMSGFTARWKYYFIWSISEASIIISGLGFSGWAPTDPPKPRWDRAKNVDILGVEFAKSAAELPLVWNIQVSTWLRHYVYDRLVPKGKKAGFVQLLATQTTSAVWHGLYPGYIIFFIQSALMIAGSKVIYRWQQATPPTKSFIKKIFVFMNFAYTVLVLNYSCVGFMVLSLHETISAYGSVYYIGTIVPILFFLLGYIIKPTRSARSSKARKDL

SEQ ID NO: 10 - protein, 318 aa, Bernardia pulchella
MGSHASKQVARRKAISTEKKTLCDLQGSCGEAYPGSDYHPPDRKNWMSGLGPEKLHINKIVWPGTHDSATNKIGFPLISRPFAQCQSLSIYKQLCLGARVVDIRVEENRRICHGILKTYSVDVVMNDVKKFLSETQSEIIILEIRTEFGHEDPPDFDKYLEEQLGEYLIHQDDHVFEKTVAELLPKRVICVWKPRKSPQPKHGSSLWSAGYLKDNWIDTDLPSTKFESNMKHLSEQAPVTSRKYFYRVENTVTPQADNPVVCVKPVTNRIHGYARLFITQCFAKGCADKLQIFSTDFIDEDFVDACAGVTYARIEGKA

SEQ ID NO: 11 - protein, 421 aa, Bernardia pulchella
MFACFADYRSLCRAHLAIGYLYLLLSSSSFIIISHAQVLESCTAATNCGAGLFCGNCPALGKNQPICTRGQAVIPTTVIDGLPFNKYTWLVTHNSFSIVDAPPLPGVQRLTFYNQEDSVTNQLRNGVRGLMLDMYDFNNDIWLCHSFRGQCFNFTAFGPAIDTLREVEAFLSENPSEIVTIIIEDYVHTPKGLINLFTNAGLDKYWFPVSKMPKKGEDWPTVTQMRQDNHRLLVFTSIASKETEEGIAYQWRYMLENESGDPGVKPGSCPHRKESKSLNSKAASLILENYFPTYPVESEACKEHSTPLAQMVGTCYKAAGNVMPNFLAVNFYMRSDGGGVFDALDRMNGQTLCGCSTVMACQAGMPFGSCKNIAAPSTSPVITTTGSFTGSVQFSKSASTILSPCYLLLYLLSFSSMAFLL

SEQ ID NO: 12 - protein, 398 aa, Bernardia pulchella
GCRLTAPVELIKCLRVIKENAFIASEYPVVITFEDHLTADLQAKVAKMETVKQTYGDMETLFCPKTDQMETENFPSPQQLKNKVLISTKPPKEYLEAKDKNSQHKSRSDDDKEEEQEHVPDEDEEGTVSEYRNIIAIHAGKPKGSSENMETLIVHPNKVRRLSLSEQELENTVKTQGDEIIRFTQRNFLRVYPKGLRLDSSNYNPFIGWAHGAQMETVAFNMETQGYGKHLWVLQGMETFKANGGCGYLKKPDFLLDPNCHPTKKILKVKVYMETGEGWDLDFHRTHFDRYSPPDFYVKIWVVGSPADKAKKKTRVIEDDWLPVWNEEFEFELTVYELAVLRIEVCEYDTSGKPDFGGQTCLPVSQLRTGIRAVPLHDSKGVQLKHVRLLMETNFQIL

SEQ ID NO: 13 - protein, 110 aa, Bernardia pulchella
ILMHFSPPDFYARVGIAGVPDDTIMKKTKTLEDNWIPVWNEEFEFPLTVPELAVLRLEVHEYDMSEKDDFGGQTCLPVSELRKGIRAVPLHDRKGVKYDSVKLLIRFDFV

SEQ ID NO: 14 - protein, 807 aa, Bernardia pulchella
MEPKLLHGTLHATIYEVDKLHGEGGHFFRKLVENIEETVGFGKGVTKLYATIDLEKARVGRTRILENEQSNPRWYESFHIYCAHEASNVIFTVKDDNPIGTTVIGRAHLPVDEIINGEEVDRWVEILDEQGEPLRHGSKIHVKLQYFGVDKDRNWGRGIWSPKYPGVPYTFFSQRQGCKVSLYQDVHIPDKFVPKIPLAGGKYYEPHRCWEDVFDAITNAKHLIYITGWSVYAEIALIRDSRRPKPGGDITLGELLKKKASEGVRVLMLVWDDRTSVGLLKKDGLMATHDEETEHYFQNTDVHCVLCPRNPDDGGSFVQDLQISTMFTHHQKIVVVDSALPSGDPERRRIVSFVGGIDLCDGRYDTPFHSLFRTLDTSHHDDFHQPNFAGASIEKGGPREPWHDIHSRLEGPIAWDVLFNFEQRWRKQGGKDLLVQLRELEDVIIPPSPVLYPDDFEAWNVQLFRSIDGGAAFGFPETPEDATRAGLVSGKDNIIDRSIQDAYINAIRRAKNFIYIENQYFLGSSFGWAPDGIKPEDINALHLIPKELSLKIVSKIKAGERFTVYVVVPMWPEGIPESGSVQAILDWQKRTMEMMYKDIIKALKEEGIMADPRNYLTFFCLGNREVKKSGEYEPSEKPEPDSDYIRAQQARRFMIYVHTKMMIVDDEYIIIGSANINQRSMDGARDSEIAMGGYQPHHLSTRQPARGQIHGFRMSLWYEHLGMLDDSFLIPQDEECVRKVNQMADKYWDLYSSETLEHDLPGHLLRYPIGVASEGDVTELPGMEFFPDTKARVLGAKSDYLPPILTT

SEQ ID NO: 15 - protein, 501 aa, Bernardia pulchella
MSQTKPARNFPSISECTGSSYESIAADLDGTLLLSSSSFPYFMLVAVEAGSLLRGLVLLLSLPFIIISYFFISEALGIQILIFISFAGVKIRDIELVSRAVLPRFYAADVRKDSYEVLDRCKRKVVVTANPTIMVEPFVKDFLGGDKVLGTEIEVNPKTKRATGFVKKPGVLVGKWKKLAVMKEFGDESPDLGIGDRKTDHDFMSICKEGYMVRRTKSAVSLPREQLKSRIIFHDGRFVQRPDPLNALVTYIWLPFEFILSIIRVYFNLPLPERIVRYTYELLGIHLIIRGTPPPPPSRGTQGNLYVCNHRTALDPIVIAIALGRKVSCVTYSVSRLSRFLSPIPAVALTRDRAADAARITSLLQKGDLVVCPEGTTCREEFLLRFSALFAEMSDRIVPVAVNCKQSMFYGTTVRGVKFWDPYWFFMNPRPTYEVRFLDRLPEEMTVKAGGKSSIEVANYVQKVLGDVLGFECTGLTRKDKYLLLGGNDGKVESMHTSKSK

SEQ ID NO: 16 - protein, 459 aa, Bernardia pulchella
MASPNSVKILEVHHVSPATVSAESSTESSLPLTFYESNWLKLPPTQNLFFYKLTDSTSFHSTIFPTLKHSLARTLDHFRPVAGHLTWATDEPRPMIQFSPNHGVSLTLAESSNTNFNQYISAKIHEAETARHFVPELHISDTYAWTMSVQITLFPNKGYCIGVTTHHAIFDGKSALMFLQAWAYITNQVINNADTFCLATGLAPSFDRTVITDPGEFESLYLNHWLTINKLESRSNPKCLKVSNFFLGIPNDFVRSSFYLSPENIKKIKERVVKFKPTQQSNVSTFVITCAYALVCMVRSVRARNQKVAFVFAVDCRNRSVINPKIPENYFGNALILHDVIVEAEDFMGENGVATVAKKISEYINGLEKGLLDGIKDRMERLSRVGDDLLKFGIAGGTRLAFYKMDFGWGNPVKVEIPSINVNALSIMEGRDGHGVEIGLGLKKHEMDEFASLFAQGLN

SEQ ID NO: 17 - protein, 502 aa, Bernardia pulchella
MDSGGEIRIRQTRRLPDFLQSVNLKYVKLGYHYLISNLLTLCFIPLIIITSIEASQMNLDDLRHLWLHLQYNLVSIIICSAFLVVGLTVYIMTRPRPVYLVDYSCYRAPDALKAPFDRFMEHSKLTGDFDESSLEFQRKILERSGLGEETYVPEAMHYIPPRPSMAAAREEAEQVMFGALDNLFANTNVNPKNIGILIVNCSLFNPTPSLSAMIVNKYKLRGNIRSFNLGGMGCSAGVISLDLAKDLLQVHRNTYAVVVSTENITQNWYFGNKKSMLIPNCLFRVGGAAVLLSNRSRDRRRAKYRLVHVVRTHRGADDKAFRCVYQEQDDVGKTGVSLSKDLMAIAGEALKANITTLGPLVLPISEQLLFFATLVVKKLFNKKMKPYIPDFKLAFDHFCIHAGGRAVIDELEKNLQLLPAHVEAFRMTLHRFGNTSSSSIWYELAYVEAKRRMRKGNRVWQIAFGSGFKCNSAVWEALQNVKPSHSGPWEDCIDKYPVTLSV

SEQ ID NO: 18 - protein, 440 aa, Bernardia pulchella
MADNHKNEVNITCKSHVKPNRKIGRKECQLVTFDLPYLAFYYNQKLLLYKKSDEHVFEDVVEKLKDGLRVVLEDFHQLAGKLGKDEEGVFRVEYDDDMEGVEIIEAVANDISIDDLIVDEGTTSFKDLIPYNGILNLEGLHRPLLAVQLTKLKDGIAMGCGFNHAILDGTSTWHFMSSWAEICRGSTSVSVPPFIERTKARDTRVKLDLTLPTDSSSNGEPNPVPQLREKVFKFSETAIDKIKSMVNVNPPSDGSKPFSTFQSLAVHIWRHVTNARELKPEDITVFTVFADCRKRVDPPMPESYFGNLIQAIFTATAAGLLTMQPPEFGASVIQKAIESHNAKAIDERNKEWESAPKIFQFKDAGVNCVAVGSSPRFPVYDVDFGFGKPESVRSGSNNRFDGMVYLYQGKNGGKSIDVEISLEAGVMEKLEKDKDFLIQL

SEQ ID NO: 19 - protein, 461 aa, Bernardia pulchella
MDKAINRQQIILDHLRPSSSSHNYESSLSASACLAGDSAAYQRTSVYGDDVVIVAAHRTALCKSKRGGFKDTYADDLLAPVLKALIEKTNLNPSEVGDIVVGTVLAPGSQRASECRMAAFYAGFPEIVPVRTVNRQCSSGLQAVADVAAAIKAGFYDIGIGAGLESMTINPMSWDGDVNPKVKAFEQAQNCLLPMGVTSENVAHRFGVTRQEQDQAAVESHRKATAATAAGKFKDEIIPVSTKIKDPKTGEEKHITVSVDDGFRPNASLSDLAKLKPVFKKDGTTTAGNSSQVSDGAGAVLLMKRSVAMRKGLPVLGVFRTFAAVGVDPAIMGVGPAVAIPAAVKAAGLELKDIDLFEINEAFASQFVYCRKKLELDPEKINVNGGAMAIGHPLGATGARCVATLLHEMKRRGRDCRFGVVSMCIGTGMGAAAVFERGDACDDLCNARKVEANNLLSKDAI

SEQ ID NO: 20 - protein, 508 aa, Bernardia pulchella
MSTQNSNHNLPNFLLSVKLKYVKLGYHYLITNAMYLLLVPVLAIFSAHLSTLTVSDFVQLWNQLKFNFVSVTVCSGLMVFLSTLYFTSRPRKIYLVNFSCYKPEDSRICTREMFMERSKLAGKFTEENLNFQKKILERSGLGQKTYLPEAVMRVPPNPCMAEARKEAEMVVFSAIDELLEKTGVKAKDIGILVVNCSLFNPTPSLSAMIINHYKLRGNILSYNLGGMGCSAGLISIDLAKQLLQVQPNSYALVVSMENITLNWYFGNDRSMLVSNCLFRMGGAAILLSNKSSDRRRSKYQLIHTVRTHKGSDDKCYNCVFQKEDQSEQKTIGVSLSKDLMAVAGEALKTNITTLGPLVLPMSEQLLFFATLVARKVFKLKIKPYIPDFKLAFEHFCIHAGGRAVLDELEKNLELSEWHMEPSRMTLYRFGNTSSSSLWYELAYSEAKGRIRKGDRTWQIAFGSGFKCNSAVWKALKNINPDNEKNPWIDEIDEFPVHVPRLVSIFTSS

SEQ ID NO: 21 - protein, 195 aa, Bernardia pulchella
TDTSAATLEWAMSLLVMHPEVLRKARAELDRVVGEERLVEESDYSKLPYLKNIIDETFRLYPTAPLLVPHESSDDCVIGGYNVRKGTMLLVNAWAIHRDPTLWKEPTSFRPERFDGEETDTYKLIPFGIGRRSCPGAGLAIKLVSVTLAALIQCFEWETVGEQIIDMNEGTGLTMPKAQPLEAMCRARESMINVP

SEQ ID NO: 22 - protein, 383 aa, Bernardia pulchella
MGAGGRMSVPPSPKNVESDILKRVPHSKPPFALGQIKKAIPPHCFKRSIPHSFSYVVQDLTIAFLFYYIATNYFHLLPHPLSFVAWPIYWAVQGCVLTGVWVIAHECGHHAFSDYQLLDDIVGLVLHSCLLVPYFSWKHSHRRHHSNTGSLERDEVFVPKQKSSIRWYSKYLNNPFGRVLTLTVTLTLGWPLYLAFNVSGRPYDRFACHYDPYGPIYNDRERIEIFISDVGILAVTYGLYRLAVAKSLAWVICVYGVPLLVVNAFLVLITFLQHTHPSLPHYDSSEWDWLRGALATVDRDYGILNKVFHNITDTHVAHHLFSTMPHYHAMEATKAIKPILGEYYQFDGTPFIKAMWREAKECVYVEQDDGDQSKGVFWYNNKF

SEQ ID NO: 23 - protein, 312 aa, Bernardia pulchella
NYFHLLPSPLCYIAWPVYWVFQGCVLTGVWVIAHECGHHAFSDYQLVDDIVGLILHSALLVPYFSWKISHRRHHSNTGSLDRDEVFVPKPKSKIAWYFKYLNNPLGRMLTLVTTLIGGWPLYLTLNASGRHYDRFACHYDPYSPIYSERERALICISDIGIFITSFVLYQVFMLKGLSWVICIYGVPLLIVNAFLVMITYLQHTHPALPHYDSSEWDWLRGALSTADRDYGVLNKVFHNITDTHVAHHLFSTMPHYHAMEATKVIKPILGEYYRFDGTPIYKALWREAKECMFVEPDEGTRDPGVFWYRNKF

SEQ ID NO: 24 - protein, 383 aa, Bernardia pulchella
MGAGGRMSVPPSPKKVESDVLKRVPHSKPPFTLGQIKKAIPPHCFKRSIPHSFSYVVQDLTIAFLFYYIATNYFHLLPHPLSFVAWPIYWAVQGCVLTGVWVIAHECGHHAFSDYQLLDDIVGLVLHSCLLVPYFSWKHSHRRHHSNTGSLERDEVFVPKQKSSILWYSKYLNNPFGRVLTLTVTLTLGWPLYLAFNVSGRPYDRFACHYDPYGPIYNDRERIEIFISDAGVLAVTYGLYRLAVAKSLAWVICVYGVPLLVVNAFLVLITFLQHTHPSLPHYDSSEWDWLRGALATVDRDYGILNKVFHNITDTHVAHHLFSTMPHYHAMEATKAIKPILGEYYQFDGTPFIKAMWREAKECVYVEQDDGDQSKGVFWYNNKF

SEQ ID NO: 25 - protein, 57 aa, Bernardia pulchella
YKMDFGWGNPVKVEIPSINVNALSIMEGRDGHGVEIGLGLMKHEMDEFTSLFAHGLN

SEQ ID NO: 26 - protein, 84 aa, Bernardia pulchella
FGTRERLSRVGDDLLKFGIAGGTRLAFYKMDFGWGNPVKVEIPSINVNALSIMEGRDGHGVEIGLGLMKHEMDEFTSLFAQGLN

SEQ ID NO: 27 - protein, 79 aa, Bernardia pulchella
MDKAINRQQVILDHLRPSSSSHNFESSLSASACLAGDSAAYQRTSVYGDDVVIVAAHRTAICKSKRGGFKDTYADDLLA

SEQ ID NO: 28 - protein, 168 aa, Bernardia pulchella
MDSGGEIRIRQTRRLPDFLQSVNLKYVKLGYHYLISNLLTLCFIPLIIITSIEASQMNLDDLRHLWLHLQYNLVSIIICSAFLVVGLTVYIMTRPRPVYLVDYSCYRAPDALKAPFDRFMEHSKLTGDFDESSLEFQRKILERSGLGEETYVPEAMHYIPPRPSMAAA

SEQ ID NO: 29 - protein, 195 aa, Bernardia pulchella
NRPSCGYMPLTKTSPGSSAAFTIPFLRHIWSWGGLTPASRQNFANLLASGYSVIVIPGGVQEMFYMKHGSEIVFVKSRRGFVRLAIEMGKPLVPVFCFGQSNAYRWWKPQGKTVLRIARAMKFTPILLGGIFRGPLPLRHPMHVVVGKPIEVEPNPQPTVEEVAEVHNQFVTALKDLFERHKARVGYPDLTLEIF

SEQ ID NO: 30 - protein, 516 aa, Bernardia pulchella
MAAAAATTRSSPKFLIVDPKKGKKRDIFKYLVENDVKSGMNFLDSSEDGVKGGAAVDHRSLLLVSIIIIRILSFLEIPLKLLGYIVDFFLNFISQNGGFFGIFINFLHGKLVIPKRGSDNFISTIGQLDGRIHLYKTPILSEQVDDSIATDNSNIKSGLGNRYLMDLSIMAAKLAYENAKVVQTVVDRHWKMHFEVFYNCWNENQKQYNTQAFIFCDKPKDANLIVVSFRGTEPFNAQDWNTDFDFSWYEIPKVGKIHIGFLEGLGLGNRSDARSFETHLQKQHQVPPGWHSEGTAIEWAKRSAYYAVAIKLQSLLQEHKNAKFVVTGHSLGGALAILFPSILVIQEETEMLQRLLNIYTFGQPRVGDEKLGNFMESHLNYPVTRYFRVVYCNDLVPRVPFDDKIFAFKHFGTCLYYDSHYFGRFMDEEPNRNFFGLSHIIPMRMNALWEILRSFMIGHTHGPEYQESWFCTVARVAGLLLPGVSAHLPVDYVNSVRLGKERVPPLESLKSFARQL

SEQ ID NO: 31 - protein, 261 aa, Bernardia pulchella
MAATTAENSKNESDVPPSFLIVDPKKGRKRDIVKYFVKKDAKSGMSFLDSSSEGIKGSAAIDHRWILLVSIVLRRILALIATPLKYLGYVIDFFLNLISQNNGLSGIFTNFLHGKLRVPQRGSDKFLSTIGQLDGRIDLYRTVILAEQVDNSIANDPNIRSELGNRYLMDLCIMAAKLVYENENVVTNIVNNHWKMNFEAFYDCWNPGQNESNTQVFMFTDKPKDANMIVISFRGTEPFNALDWSTDFDFSWYWIAKIGRI

SEQ ID NO: 32 - protein, 38 aa, Bernardia pulchella
GCCSWYSAHSPIDYVNSVRLGKERVAPLASLKSFTRNL

SEQ ID NO: 33 - protein, 95 aa, Bernardia pulchella
MAPARCMSLNLAVRYADVINSVVLQDDFLPRTTTALEDVFKSLFCLPCLLCLMCLKDTCTFEEKMLRDPRRLYAPGRLYHIVERKPFRIGRFPPI

SEQ ID NO: 34 - protein, 119 aa, Bernardia pulchella
MFVQSYAYPSRCGLTKEFMLKFMLICSFCEQGKEFKIKHKHINHHLPVYSHTLAKILVEYASAVYMSDLNELFTWTCSRCCDMTEGCEIIELIVDVEHCLQESHDMVYKVVQFGCVPYA

SEQ ID NO: 35 - protein, 372 aa, Bernardia pulchella
MFRELKTMPFHSSKAPTLFTRFLVMVHILAWTNKTTALVKLPANVTAPPAVIVFGDSIVDAGNNNNIKTLIKCNFPPYGLHFYGGIPTGRFSDGKIPSDIIAEELGIKATLPAYLDPNLLPQDLITGVTFASGGCGYDPITPKLVSVISLDDQLNHFNDYKEKVKAIVGEERANFIITNSLFLVVAGSDDIANTYFDLRARKAQYDVPAYTDLMVDSASTFVQNLYKMGARRVGVFGAPPIGCVPSQRTLAGGPKRECASNYNEAAILFNSKLATQLESLTATLPQSKIVYVDIYQPLLDFIQNPQQYGFEVADKGCCGTGVLEVAVLCNQVTPVTCANVFNHLFWDSYHPTERAYNILITQLLVKYVQKFF

SEQ ID NO: 36 - protein, 280 aa, Bernardia pulchella
YTASPSSQSSRVSRAISLAKNDAHKTGWTVYLLSWILFPLRFMLLLPFHLCGLFYKRRSTAPSMRRSHKPLRVHSIRRIYNVKDNVIHRTTDRRRGVIEDLHLAIEIVIEAIFGYFHKAAHFFLSPSEAFRVVHKWFSSQSSYNEEIQNGAYDASVPTATLGENDPAITEKNTTFNHLLNTDARTCQDVITELGYPYEVIRVITSDGYVLLLERIPRRDSRKVVYLQHGILDSSMGWVSNGIVGSPAFAAYDQGFDVFLGFSGFSFQEPVDRTYLPEILE

SEQ ID NO: 37 - protein, 160 aa, Bernardia pulchella
HEAHPHTMPPESTPPNFWGDMPEDEYYASQGVINSQSYFQTVNGKLFTQSFIPLDQKVKATVYMTHGYGSDTGWLFQKICISYATWGYAVFAADLFGHGRSDGLRCYMGDMEKIAATSLSFFKHVRYSDPYKDLPAFLFGESMGGLATMLMYFQSEPNMW

SEQ ID NO: 38 - protein, 182 aa, Bernardia pulchella
MGGAVTLKVHLKQPNAWDGMILVAPMCRIAEDVKPPPPILKAVTILSRFLPKAKLVPQKDLEEVFIRDLKTRKMADLNVIGYNGQMRLKTAVELLKATEEIEAQLEKVSSPLLILHGAADKVTDPQVSQFLYERASRKDKTIKLYEEGYHCILEGEPGDRIFGIFDDMISWLDLRSSTLSRK

SEQ ID NO: 39 - protein, 131 aa, Bernardia pulchella
NPRLFFTHHLLLLPLLLSSPLLFFFFFFFFFFFFHCHERRHSHFFFFHCHERRHSHFFFFFFLFRFICFTFWLRASPFGCWFVCFGLTVPLVREFDRDDNLNNDVSEELSDEKTSVPYEPVYSDEQARLNI

SEQ ID NO: 40 - protein, 160 aa, Bernardia pulchella
MESVLKKWCWVVCVVLCLVLDVGGQQVPCYFIFGDSLVDNGNNNQLQSLARANYMPYGIDFPGGATGRFSNGKTTVDEIAVQLGFTNFIPPYATARGQQILGGVNYASAAAGIREETGQQLGGRISFRGQVRNYQNTVSQFVNLLGDEDSAANYLKQCIF

SEQ ID NO: 41 - protein, 144 aa, Bernardia pulchella
MGDSRCASRIISFTLLLFACSCNAQNLADCRFEAMYQFGDSLSDTGNSIVEVPQAYHSRLPYGMTIGQATGRPSDGYLMIDYFALSAGLPLIQPFENPKSNFTYGADFSVAGVTALPWQTLSKLGLALGYSNSSVPVQIEWFKK

SEQ ID NO: 42 - protein, 175 aa, Bernardia pulchella
MATNKSFFFNIFSVLFIFLFFSQSHSIDFNFPAVFNFGDSNSDTGNLIAAGFESINPPYGQNYFQKPSKRYCDGRLTIDFLLDAMDLPFLNPYLESIGLPNFHRGCNFAAAGSKILPATASSVSPFSFGIQVNQFLRFRFRALQLLAKGKKLERYLPAEDYFQKGLYILDIGQND

SEQ ID NO: 43 - DNA, 1443 bp, Bernardia pulchella
gccgccacattttcgctggatttcggtgaggtagaaatcgccatggcaccgtgagaggccatctatatctatgtggttaattatactaatgtttagaaaggaaggaagaaagaagcagacggaaaaccaattcagataaaataagaatcgaaacaaacgacttccgattgtcaaaaccgttgatggttattgaggtgagagtattgattcattagaggaggagcagtaagcatggaggaggagaaaatgaagaagaaagaagagggattgagagtgattaacgctagagatgtttataaaacaaacatgtttcactccctcttatcgctcatgctatggatcggctccatccatttcaatttcttccttgtcttcatctcttttatcttccttcccgttcccaaatttctcttcgtcgtaggattgcttttggttttaatgttcattcccattaacccacgcagtaatttcggccttcgtctctgcaggtacatgagtagacatgcttgctcgtattttcccatcactctccatgtcgaagacatgaatgcatttcgttctgatcgtgcttacgtttttggctatgagcctcattctgtttttcctcttggtgttgccatactttctgatcacatgggtttcatgcctctacctaaaattaaagttcttgcaagtagcactattttccgcacaccatttttaagacatatatggacatggtgtggtcttgctccagctacgaggaaaaattttacttcccttctagcatctggttatagttgcatcgtgattcctggtggagttcaagagactttttatatgaagcatggttctgagattgcgtttcttaagacaaggcgaggatttgtccgactagctattgagatgggcaaacccttggttccagttttttgttttggtcagacaaatgtgtacaggtggtggagacctggtggcaagttagttatgaaatttgctcgaatcattagatttgctccactttttttttggggtgttctcggatctcctttacctttgcctcatcctgtgcatgttgttgtgggtaggcccattgagctgacgcaaaatccacagcctacaatggaagaggtcgcagaagtgcacagtaaatttgttgcagcgcttagagatctttttgagaggcataaagatcgagttggctgcggagacattgcacttgaaattcattgatagtagactttgtttggtttattgtacccacttttatattcatcgccaatccttcacccaagtgtttgaaatgtgagtattgactattcgattttttttttcattatttgcatgcagaaataagttttgaccagaatatatcaacttaaattttgttttagaaaagaaatgttatcccttatctataatataataaaacaaatggaaattatgaaaaaaaaaaaaaaaaaa

SEQ ID NO: 44 - DNA, 2028 bp, Bernardia pulchella
ctctatctttgcatgacataacactattttgaactccttctttctccacttatacgcaccgtttctgatctccaatgacgattctcgaaacaccggacacacttatatcttcatcaccgacagcgacggctggcgtcatcacttccgatcttaatctctcacttcgtcggagaagatggacgtcatctaactccgacagtgcgattgctgaattagcttcgaagattgacgacaaagaagaaaacggtggcctcatcgacgaggtgaaatctaaagaagaacgtagagaaaatttaagcatgaatcctattgcatcgacggcggcggttacggaattagaaacgttaacaagcaatgcaaaagagtccgtcgttaataataataatgataatgatgataatgatagtagtaataaatttgaaaatagtgaaaatcatgaaagaggaatcgatattaaatttacttacagaccttctgttccagctcatcggtctcataaagagagtccacttagctctgatctaatatttaaacaaagtcatgcaggtctcttcaatctttgtatagtggttcttgttgctgttaatggcagacttatcattgagaatctaatgaagtatggttggttaattaagaccggcttttggtttagttctagatcattgagagattggcccctttttatgtgctgtctttctctcccagtattccctcttgctgcctatctagttgagaagatggcatatcgaaaatgtatatctgcacctattgttattttccttcacgtgatcattacctcagcagctattttatacccggtttctgtgattctcagttgtgaatctgctgttttatctggtgtcacattgatgctgtttgcttgtattgtgtggttgaaattggtttcttatgcacatacaaattatgacatgagagcagttgccgattctgttgataagggacatgcatctaatactttgagtgcagagtattcccatgatgtgagcttcaagagtttggtgtacttcatggtagctcccacactatgttaccagacagtttatcctcgcacagcctctattcggaagggttgggtgtttcgtcagtttgttaaactgataatattcacaggtttcatggggtttatcatagaacaatatatcaatcctattgtccaaaattcacaacaccctttaaaggggaatctcttatatgccattgagagggttctgaagctctcagttccaaatttatatgtgtggctctgtatgttctactgcttctttcacctatggttaaatatacttgctgagctacttcgctttggtgatcgagaattctacaaagattggtggaatgcaaaaactgttgaagagtattggaggatgtggaatatgcctgttcataagtggatggttcgccatatctatttcccatgcctacgccataaaatacctaaggaggtggctctagttattgctttctttgtttcagctgtatttcatgagttgtgcattgctgttccctgccacatatttaagctttgggctgcttttgggataatgtttcagattcctctagttctgatcaccaaataccttcaaaataagttcagaagttcaatggtgggaaatatgattttctggttcttcttctgcatccttggccagcctatgtgtgtacttctgtattaccatgacctaatgaatcgcaaaggtaaaaactgaatcaatatgaaagcaatccatggcagcttgagatcagtttggatatagataggggtttgcttccgattcgggccgtaccgcattattgccattactacacctgatttgacaagagtcggtttaagttagtcaggctctggctacatgcaattttgaaagtacaaacccttggcagagcatggtgtgatattgtaatgtaaatgtacggactctgtcgatcaagtgagaaactgtaaatgtatcatgttcatttttattaatggtatttcatttgatttttattaaaaaaaaaaaaaaaaaa

SEQ ID NO: 45 - DNA, 1188 bp, Bernardia pulchella
gaagcataaatatttcacaggagacgacgacgacgacgacgaggacgacgagagagagagagagtgatagagatgtcggtgatcggaggttcatctaaggttctattgagaccgagaaaagctaataaggtttcgagtaactgtttttttagagataacggatatttgaattattattataattacaacgaaggagttgtgagatgtggaggcgattgcagcaaatcgatcaagaaaaaattgaagttagtaaaatcgttaacgaaggatttatcgatgatttctgatatggcggattttcaacttcatcaagctcaggtcacgtctcttcaggatgcttctcgagcgttgatgcagcagcttgaagaattgaaagcaaaagagaaggaattagagagacagaagaaagaagaaaagaaaactaaccgtaaaccaattgaaacgatgatgatggattctgaatcatctacgacgtcatcttcctcatccagcgaaagcgattgcggtgatgaagaagtgattcgtatgagccgattgaattacgactccattgctgttgcagaaccagtttcaaccctaacgatgctaccaaacaagcaagaacaaacctgcgattgcggtgatgaagaagtgattcgtatgagccgattgaaatacgactccattgctgttgcagaaccagtttcaaccctaacgatgctaccaaacaagcaagaacaaacctgtgtcatttcaagcaataaaatgaacaaaccaaatggattacctgtaggagaattaactgaaaagaaaattgaggtatgtatgggtaataagtgtaagaaatcaggtagtgtggcgttgatggatgagttccaaagggttatgggttcagagtctgtggtttgtgggtgtaaatgtatgggtaaatgtagagatgggcctaatgtgagagttgtggattctgctgcaagttcagtgagctctctgtgtattggcgttggtttgggggatgtgggtgatattgcgaataggattttggggaaggagaaagttttaggcctcgccatagcgtcgccgtaggctcgtgttgtattattagccgatgctgctgttatataagcaaacttggtatcgataacgttcatgctactgagaatgaaaatgtgtatatattagatatttgaataaaaaaaaaaaaaaaaaaaa

SEQ ID NO: 46 - DNA, 734 bp, Bernardia pulchella
cttcttctctccataattgatcggacgcttttttgcttgcaaaaccaaagcttctttcattgacaagatcatggagaattatatataccagtcggtggcttatgttatcttactttccttcttttcttcttctaatcatgctgttgcccttaacattggtgtccaaacagcaaattcagctatcactttgagtaaagaatgcagtaggaaatgcgagtcagaattttgttcagtgcctccatttttgaggtatgggaagtactgtggactgctatacagcggatgtccgggagagaaaccatgtgatggacttgatgcatgttgtatgaagcatgatgcttgtgttcaagccaaaaacaatgactatttaagtcaagagtgtagtcaaacattcataaactgcatgaacagttttaagaagactggaggccatactttccaaggaaaccaatgccaggttgatgaagttattgaggttatttctttagtcatggaggctgctttattggctggtagataccttcataagccttaattattatttcaaatttgttaatttgaataattaggataagttactcctatcttccctcttttactttccatttttcataatttaatgatccttcccttttgtaatttcatcttactactttaggatgttcaactgttcttcattttttattgacttataaaatcacactcaatgtttatcaaaaaaaaaaaaaaaaaaa

SEQ ID NO: 47 - DNA, 2083 bp, Euphorbia lagascae
ctcgttttccctcgtcgtccatagcctagccggctcatttcgcgctctctgctctcccttccccgccgttttgttctgcttaaccatacaagaatgtccgactcttatttggggttagggtttggctctatactctgcttgtagtcgacgatcacactcggtctctcttcatgaaagaaataataattaagaaatacagcagctcttcgttctttcacttattcaatcctttttctttctttgttttattagcgactactccgagatgtcaattttgaaacggaggtcatcgaaagtacggagttcttcagatttatctgattttcagaacgacgacgatgataataaaaaagagagggagaagccgaaaaggaggcaaactggaagagggaggagagggaagaaatggacatgtttggacagttgttgttggttcatcggttttatatgctcgatgtggtggtttttactttttttgttcaatgcaatgccttcttctttccctcagtacgtcacagaggctatcacggggccttcgtcggatccgcccggcgtgaaattgaagaaggaaggcctacgtgtgaagaacccggtggtttttgttcctggaattgttacaggtggccttgaactgtgggagggacaccagtgtgctgatggactgttcaggaagcgactttggggtggggcatttggtgaagtttacaggagaccattatgctgggttgagcatatgtcactggacaatgaaactggactggaccctccaggtgtcagggttaggcctgtatctggacttgtggcagctgattattttgcgcctggttattttatctgggcagttttaattgctaatttggcccgccttggatatgaggagaagaccatgtatatggccgcatatgattggagactatcatttcagaacaccgaggtcagggaccaaactctaagcaggataaaaagtaatatagaactaatggtggctactaatggtggggaaaaggttgttgttattccacattcaatgggtgctctatattttctgcatttcatgaaatgggttgaagcacctgctccgatgggtggtgggggtggagcagattggtgtgctaagcatataaaggctgtcatgaatattggtgggccctttttaggtgtgcctaaagcagtgtctggggttttctctaatgaagccagggatattgctgctgccagggcttatgcgcctgtgtttttggacaaggatgtctttggtcttcaaactcttcaacatttaatgcggatgactcgcacatgggattcgaccatgtccatgttaccaaaaggtggggatacgatctggggtgggcttgattggtcacctgaaggaatctataactgtggtgctaagaaaccgaagaacaatacaacaaactctgtaggtcaaaccggaaaagggacttcaagttttaaagacggagtgaactatggccgaattatttcatttgggaaggatgtggctgagcttcattcaaccaaaattgaccggaaagatttcagggacgtctttaggggtaataaagatacaaacagctgcgacatctggacagagttccatgaaatggatattgatgctctgaaagctgttacagattacaaagtttatactgctgattcaatattggatcttcttcattttgttgctcccaaacttatggcacgaggagatgttcatttttcacatgggattgcagacaatttggatgatccaaaatatggacactataaatattggtcaaaccccttggaaacaaagtgagagtttccaccttcacttttcatgactttttgcttttgatttaccccttggaaacaaagtgagagtttccaccttcatttttcacgactttttgcttttgatttagtgcattaggatttggttctatttttctgccctgggtacaagttccatgaaccaaacaaactcatgtattctacttccagaaaatgcttctagttagctatttgtatggatttccttcatacctagattataataatcatcacaggaagaaactcaaaaaaaaaaaaaaaaaaaaaa

SEQ ID NO: 48 - DNA, 2665 bp, Bernardia pulchella
cttttatttttctagagaaagaattttcctttttctaagctgtttttaaattttatttcttctttattttatacagaataaaaaatttgtgttttgtaattcttgattagatcatcaattagcttgctgggtctgatttagtttaatttactttaattttctgcattaaagccttgaaagctacaagtttttggtttaaactcagaaatgccattaatcaggagaaaaaaacccactaaggaaacgattgaagattcaacagcaagcaaggaagaagaaaaagggaaagagcaagaagaagaaaaagaagaagaagacaaaaacaataaaaagaaatacccaaataaaaggaacagtcaaatcaagccaaaatggtcgtgtgtagataactgttgttggtttgttggttgtatatgcatatcatggtgggttttactgtttctttataatgcaatgcctgcatctttacctcagtatgtaaccgaggctattaccggtcctttacctgacccacctggtgttaagctgagaaaagaaggattgaaagctaaacatccagtggtttttgttcctggaattgttactgctggccttgaattatgggaaggccatgaatgcgctgatgggttgtttaggaaaaggctatggggtggtacttttggagaagtttataagcggcctctttgttgggtagagcatatgtcactagacaatgaaactggattggatccttctggtataagggtgaggcctgtctctggacttgtagcagctgactactttgctcctggctattttgtgtgggcagttctgatcgctaacttggcacgaattggatatgaggagaaaacaatgtacatggcctcatatgattggagactttcatttcagaatactgaggtacgtgatcaaacactaagccgaatgaagagtaacgtagaacttatggttgccacaaatggtgggaataaggcagttattgttccacattccatgggtgttttatactttctgcattttatgaaatgggtcgaggcaccagctccaatgggaggagatggtggaccagactggtgtgctcggcatatcaaggcagtcatgaatattggaggtccatttttaggcgtcccaaaagctgttgctgggctcttctcagctgaagcaagagatattgcagttgccagagccattgcaccaagttttctggacaatgatatattccgtttacaaacattgcaacacatcatgcggatgtctcggacatgggattcaaccatgtcaatgataccaagaggtggggacactatttggggtgatcttgattggtcacctgaagaaggttatgttactagaaagaggaagcaaaaaaaaagtactgctgattatgcaaaccaagatggggatgaaagtgagagttcacaaagaaaatgtgtgaaatatggaagaatgatatcttttgggaaagatgtagcagaggcaccttcttctgatattgagaggatcgacttcaggggtgctgttaagggtcatagcgttgtaaacagtacctgttgtgatgtgtggacggagtatcatgaaatgggatatggaggcattaaagctgttgcagagtacaaggcttatactgctgcttctattatagacctgcttcagtttgtcgcaccaaaaatgatggagcgaggtagtgctcatttctcttatggaattgctgacaatttggatgatccaaagtacaagcattacaaatattggtcaaatcccctagaaacaacgctgccaaatgccccggaaatggaaatattttccatgtatggagttggcataccaactgaaagagcttatgtttacaagctataccctgcttcggaatgctacattccatttcagattgatacgtcagccgaggggggagatgaagatgactgtttaagagatggagtctacacggttgatggggatgagactgttcctgttttaagtacagggttcatgtctgcaaaagcttggcgtgtgaaaacacgttttaatccttcaggaattcaaacgtacaatagagagtataaccattctcctccagccaatttgctagaagggcgtggcacgcagagtggtgctcatgtcgatataatgggaaattttgcattgatcgaagatatcatgagggttgctgctggagcaacaggagaagagttgggaggtgatcaagtttattcagatatctttaagtggtctgataagatcaacttgccactatgaatcgcaaggtttcggttctcacgtaaaccatagtattgtcaagtctgattctgttcttgcatgcatcaccagctgtatgagcattcgatcctataatcttctgtcacttctgaaacttggtcattccatggtatgaaagtgattccataaaagaaaaggtttctgttcgtgactgaatttcttatcagaaagtgagtatgaagaaagcgtcagagctgactgcatgatgacaagaattacctgtaatgtagtactgcactagagttgttgtcttagctctttgctatacgattcatttgataatatttgtgcaactcttattatcccgaacatcaataattgaaaattaccgtatatttttctcaaaaaaaaaaaaaaaaaaaaaaaaaaaaaaaaaaaaaaaaaaaaa

SEQ ID NO: 49 - DNA, 2121 bp, Bernardia pulchella
atgaagacgataatacaccacaaccatgcgattgccaaatcatgagatcaattagaattagtgcagatttgtcaccgaaacaacactaaaatcaattacacacctgtaatctaaaaaaattcataagcttattcgcattcaagacccttaattcccagtcgtcgataaaaataaatcgaaacccaaatctcaattgtcaaaaagtcgccgcaacattgatgagtctcgacctgttagtgagtttggttaagttcatttttgcgtggtgtaatcagcgggagacaggggacaatgaaaagggtatgcaacagagaaattaagcggcaatggcgaagcagctgtcatataaataattggttgaatttcaagtgaattgagctcaaaaaaacttataatggaaaatgtgtaagcataataaagttaagggcaaaaatgagtatttttcccatttgaacggttacgcaggaataaaagagcaaaaacgaagaggaaaactaagaagtgaaggggaatatggggtacatagggaaacatggagaagcagcacttcgcagatacaaatacagtggagaagatcattcttatgttgctaagtatattcttcaacctttctggactcgttttgttcgcttcttccccctctggatgcctccgaacatgattacgcttacaggatttatgtttctagttgtttctgctctgcttggctatgtgtactcacctcacttggatacacctccaccaagatgggtccattttgcacatggattacttctgttcttatatcagacttttgatgctgttgatggtaaacaagcacggcggacaaactcctcaagtccattgggagaactttttgaccatggatgtgatgcgcttgcatgtgcgtttgaaagcttggctttcagtagcaccgccatgtgtggaagggatgctttctggttttgggttatttcagctgttccattctatggtgcaacatgggaacacttctttacagatacccttatcctgcccgctattaatgggcctacagaggggttgatgctaatatatgtggctcatttttttacagcaacagtgggtgctgagtggtgggttcaacactttgctaagtcttttccttttttaagttgggtgccattcattagtgaaatccaaacatatagagcagtgctgtatctaatgacggttttcggtgttataccaacagctgcatgcaacatgtccaatgtctacaaggttgttaaggcgaggaacagtagcatgtttctggcattagccatgctttatccttttgctgtgcttatgggaggcatgttattgtgggattatctctctccatctgatttaatgtgggattatcctcatttggtagtccttggaactggacttgcgtttgggtttcttgtgggtaggatgattctggcacacttgtgtgaggaacccaagggtttaaaaactaacatgtgcttttctttgttgtatctgccagttgccattgcaaatgcccttacagcgaggttgaataatggagttcctttggtcaatgagttctgggttctacttggttactgtttattcacagggggactctatttacactttgcaacatcagtcattcatgaaatcaccacttccttgggaatcaattgcttcaggataactagaaaggaagcttgaaagtgcaggttttggattttggtttgaattgctttgaagggacataaattatgtactattagttatcaaattcccagcaattcaagagcaatggaggacatttaatagttcgtagtggagttgtaacttgatcagggtaatttagtccaattatgttgttacagatttcatcaaaaaattatttaggcaatcaaagcacgtaaatcatgaataaatatatttgttttagattcttaagatggttgttgaagcttcatacaactatgatgagaacttgtagggtgggtttggagtttgtgtcattgtaaggacaaatctttgttggaaccacccacgggtgaggcatgtcatcaacgcatcatatgatatcataaaaattttggaaacacattttaggatcctcgactataaaaaaaaaaaaaaaaaaaaaaaaaaaaa

SEQ ID NO: 50 - DNA, 1622 bp, Bernardia pulchella
ctctgtgatttaacagagtttactcgttaaaacctctgattttttaagtaaaaaagaatgggactagagatggattcaatggcatcggcgataggcgtatcggtgccagtattgagatttttactctgtttcgtggcgacgattccagtgagttgcatgcatcgagtcgtacctggtagactcggtaaacatgcttatgctgctctatcaggtgttttattgtcatatttatcatttggcttctcttcaaatctccattttttggtacccatgttactgggctatgcttccatggtattgtttcgctctcactgtggaattttggcgtttattctgggctttggttatcttatcggctgccatgcttattacatgagtggagatgcatggaaggaaggaggcattgatgctactggggccttgatggtattaacactgaaagtcatatcatgtgcaataaattacaatgatggactattaaaagaggaggaattacgagaatcacagaagaaaaaccgattgattaaacttccgtctttgattgagtacattggttattgcctctgctgtggaagtcattttgctggtcctgtatatgaagtgaaggattaccttgagtggaccgaaaagaaggggatatggactcacaccccatcaccttatggggcaacggttcgagctattcttcaagctggtatttgcatggctatttatctatacctggtgccgcactatcctttatcccgcttcaatgatcctctgtaccaagaatgggggtttttgaaacggttgagttaccagtatatgtctggttttacagcacggtggaaatactatttcatatggtcaatttctgaggcctctatgattatctctacacttggtttcagtggttggactggtagttctcctccaaagccacgttgggatcgtgcaaaaaatgttgacatactaggtgttgagtttgcaaagagtgcagctgagttgccacttatgtggaacattcaagtcagcacatggcttcgtcattatgtttatgaccgacttgttccaaaggggaagaaagcaggtttcgttcagttgttggccacccagacaaccagtgctgtttggcacggattgtatccaggatacattatattctttattcagtcagcactaatgattgagggttcaaaagtcatatacagatggcaacaagctatacctccaaagaaggttcttttcaagaaaatgcttgtctttataaacatggcctacacgcttttggttttgaactgttcttgtgttggtttcatggtgctgagcttccatgaaactattgcagcatatggtagtgtatattacgttgggaccattgtgcctatagtaattttcctacttggattcataatcaaaccagcaagatctgtcaagtctaaaggccggaaggattagtgaggttgaaatctttgcttcacttgtacaacgggtcgcaaataatatcagtactttgtagctgtgtaaatttggaacctgttctttcatttccgaactgtaataggaaccaagttttaatgtgatcctcgattgtgctcgctttcagctatcatttttcaaaaaaaaaaaaaaaaaaaaaaaaa

SEQ ID NO: 51 - DNA, 1732 bp, Bernardia pulchella
caaatcttgaaatcagagcgtctttgtctaacttctcctcgaaccagagagagagagaactcgtttattaactcagtgggttaagcgagtcaactcattctgatcgtaatcattaaacaaaaccctgtgaatccaaacatggatttacacatggaatcaatggcgtcatcgatcggggtctcgatcccagtactgagatttctgctctgtttcgtagcgacgattccagtgagttttgtacaccgagtcattcctgggagactcggtaaacatgtgtacgctgcattgtcaggggctgttttgtcgtatttatcatttgggttctcttcaaatcttcattttttggtacccatgttgttgggttatggttctatggttttgtttcgttctcactgtggaattatgacatttgctttgggttttggttacctcattggttgccatgtgcattacatgagtggagatgcatggaaagaaggtggcattgatgctactggggctctaatggtgttaacattgaaagtcatatcatgcgcaatgaattacaatgatggattattgaaagaagaggatttaagggagtctcagaagaaaaataggttgattaaattgccatcattgattgagtactttggttattgcctctgctgtgcaagccactttgccggtcctgtttatgaaatgaaggactatcttgactggactgaaagaaaaggaatatgggctcgcacagagaaaggaccctcaccgtcaccttattgggcaacggttcgagctgttatgcaagctgcaatttgcatgggcatttatctatatcttgtgccatactatcctttatcccggttcactgattctgtatatcaagaatggggcttttggaaacggttgagttatcagtatatgtctggttttacagcacgttggaagtactatttcatctggtcaatttctgaggcttctattattatctctggactgggtttcagtggttgggcgccaactgatccaccaaagccacgttgggatcgtgctaaaaatgtagacattcttggtgttgagtttgcgaagagtgccgctgagttgccacttgtgtggaacatacaagtcagcacatggcttcgccactatgtttatgatagacttgtcccaaaggggaagaaagctggtttcgtacagttgttagctactcagactaccagcgctgtttggcatggattatatccaggatacattatattctttatccagtcggcattaatgattgcaggttcaaaagtcatatacagatggcaacaagctacccctccaactaagtcttttataaagaaaatattcgtgttcatgaacttcgcatacacagttttggttctgaattactcctgtgttggtttcatggtattaagcttgcatgaaaccatatcagcatatggcagtgtatactacattggcaccattgtgcctatattatttttccttcttggctacataatcaaacccacgaggtctgctagatcatcgaaagctcgcaaggatctgtgaggtttttgtcacttaagctctgcaacccggttgagtttcttcaagttataatctgaatccttacaaacatgatatggtaggacctcacatatatcacacacctttgaattgagaaataacatcaaacatttgttgtgaccatatttaataagctctaataaatatatttccatcaaaaaaaaaaaaaaaaaaa

SEQ ID NO: 52 - DNA, 1152 bp, Bernardia pulchella
tccatgcataaatgggttctcatgcttctaaacaagtagccagaagaaaagcaatttcaacagagaaaaaaactctctgtgatcttcaaggaagttgcggtgaagcttatccaggttctgattatcacccacctgataggaaaaattggatgagtggtctgggtcctgaaaagcttcatatcaacaaaattgtgtggcctgggactcatgattctgctactaataagattggttttcctctgatctctagaccatttgcacagtgtcaatctctctccatctataaacagctttgtctgggtgctagagttgttgatatcagggtggaggaaaatcgtcgaatctgtcatggaattttgaagacatatagtgtggatgttgttatgaatgatgtcaagaaatttttgtcagaaacacagtctgagatcataattcttgaaatcaggacagagttcggacacgaagatccgcctgattttgacaagtacttggaagaacaactcggcgagtacctgatccatcaggatgatcatgtgtttgagaaaactgttgcggaattgctgccaaagagagtgatttgtgtttggaagccgagaaaatcaccacagccaaaacatggaagctctttatggagtgcagggtatttgaaagataattggattgatacagatttgccatcaacaaaattcgagagcaatatgaagcatttgagtgagcaagcaccggtgacatctaggaagtacttttacagggtggaaaacacggttacaccacaggcagataacccggttgtatgtgtgaagcctgtgaccaatcggattcatgggtatgctaggctgttcataactcagtgcttcgctaaaggatgtgcagataagctccagatcttctctactgattttattgatgaggattttgttgatgcttgtgctggggtaacgtatgccaggattgaagggaaggcttgattgatcttttcatttattttgaagttgcttcctgtttatgtacattgccgctacattgatttgtgatctctgttatcatctatttgtttgctgtatctatttatttgtgtttcttcatatgtgatgtgtataaaaccgtaacggaatggaagattttgggttttgcacaaaaaaaaaaaacaaa

SEQ ID NO: 53 - DNA, 1748 bp, Bernardia pulchella
agttattgagctctgcataagcattaactggcaatgtttgcgtgcttcgcggactatcgtagcttgtgcagagcccatttagcaattgggtatctctatctcttactctcgtcatcatctttcattataatatcacatgcacaggttctggaatcatgtacagcagctacaaattgtggtgctggtttattttgtggaaattgtcctgctttaggcaaaaatcaacccatttgcacaagagggcaagctgtaattcctactactgttattgatgggttgccttttaataagtatacttggttagtgactcataactcttttagcattgttgatgcacctcctttgcctggtgttcaaagacttactttttataatcaagaagattctgttactaatcaattaaggaatggtgtaaggggactaatgctggatatgtatgattttaataatgatatttggctttgtcattcatttcgcggacaatgtttcaacttcacagcctttggacctgcaattgacaccttgagggaagtggaagctttcttgagcgaaaatccatctgagattgtgaccattataattgaggactatgtgcatactccgaaagggttgataaatttgttcactaatgctggtttggataagtattggtttcctgtctctaagatgcccaagaagggcgaagattggcccactgtgacacagatgaggcaagataatcaccgacttctggttttcacttctatagcttctaaagagacagaggaaggcattgcttatcagtggagatatatgttggagaatgagtctggagatcctggggtaaaaccgggctcatgtcctcatagaaaagagtcaaagtctctaaattccaaagctgcatctctaatcctagagaattacttcccaacatatcctgttgagagtgaagcttgtaaagagcattcaactccgcttgctcagatggttggtacctgttataaagcggcaggcaatgtgatgcccaattttctggcagttaacttttacatgagaagtgatggtggtggtgtttttgatgctttggacagaatgaatggtcagacactttgcggctgtagtaccgtgatggcctgccaggctggaatgccttttggatcttgcaaaaatattgcagcaccctctacgagtcccgtaattactactacaggaagctttacaggatcggttcagttttcaaaatctgcatcaacaatcctttctccatgttacttgcttctctacttgctttcattttcgtccatggcatttttactatgaacgggataggtgttttcctctttaggcagtgaaggttctactgcattagttagatagcaagctaccgtttgcgtagaggctgttagtgtcagcacgtattgttcagttcacatctctgccttttcagggaaggttataacataacatacttgctagaggtatgaagtgtcaatgtgaacaaagaacttttatgattcttacatagaacgaaaagtaaagacttgcaatttgcaaagttatacgagaaatatagcgtcgaggatgtatcagcattcggttcaggtacgcaatccacttgtaggcaagattgtatgcatcttttaggattgatgtttcatctcttgctctgtccaaatagatatagcaatctttaatttctatttcatgtgttttgaatattgatcaggtgaataactttatactttttgtcaaaaaaaaaaaaaaaaaaa

SEQ ID NO: 54 - DNA, 1224 bp, Bernardia pulchella
cgggctgcaggctcacagcgcctgtggagctcataaaatgtttgcgtgtcattaaagaaaacgcatttatcgcgtctgagtatcctgtggtgataaccttcgaagatcacttgacggcagatcttcaggccaaagttgctaagatggtgaagcagacatatggagatatgctattctgtccaaaaacagaccagatggaaaattttccttcaccacaacaactgaagaataaggttttgatttcaactaaaccaccaaaagagtatcttgaagccaaagataaaaactctcaacataagtcaagatcagatgatgacaaggaggaggagcaagaacacgttccagatgaagatgaagaggggacagtttcagaatacagaaatataatagccattcatgcaggaaaaccaaaaggttcatcagaaaatatgctaatcgttcatccaaataaagttagacgtctcagcttaagtgagcaagaacttgaaaatactgtaaagacacagggagacgaaattatcaggttcactcagcgaaatttcctgagagtatatcccaagggactgcgacttgattcctctaactataatccattcatcgggtgggcgcatggagctcaaatggtggcttttaatatgcagggttatgggaagcacctttgggtgttgcaaggaatgtttaaagctaatggtggctgcggttacttgaaaaaacctgacttcttattggatccaaactgccatcctacaaagaaaattctgaaggtcaaagtatacatgggtgaaggttgggatctggattttcaccggacacatttcgatcgctactccccgcctgatttttatgtcaaaatttgggttgttgggtccccagctgataaggctaaaaagaaaacaagagtaatagaagatgattggttaccagtgtggaatgaggaatttgagtttgagctgacagtttatgaattggctgtgctgaggattgaagtgtgtgaatatgacacatcagggaaacctgattttggaggccaaacttgtttacctgtttcccaattgagaactgggattcgagctgttcctttacacgattctaaaggagtgcaactcaaacacgtcagacttctcatgaattttcagattctatgatcccatcagtaccataatctttgatgattctacatatatatcacttaatattgtacttccaaaaaaaaaaaaaaaaaaa

SEQ ID NO: 55 - DNA, 583 bp, Bernardia pulchella
cattttgatgcatttttcccctccagatttttatgcaagagtagggattgctggagtcccagatgatactataatgaaaaagacaaagacacttgaggacaactggatacctgtgtggaatgaggaatttgagttcccattaactgtcccagaattggctgtgcttcggcttgaggtacacgaatatgacatgtctgaaaaggatgattttggaggtcaaacatgccttccagtctccgagttaaggaaagggatccgagcagttccactccatgaccgtaaaggagttaaatacgattctgtaaagcttctcatccgttttgattttgtttgagattgaacagagcgatgtgtgctaacctctgcagatgtatagatttgcaaagctttttcaatatgttccatgtgagtttgtgcgctattatacatttcacattgttggcgtgtgacataatatctgtttgtgtagtctatgggatgctgtggggaactcgcattacacattgcgctgtaaaaagtgactatcaattattagatatataaatgtgattatatgttgtaaaaaaaaaaaaaaaaaaaaaaa

SEQ ID NO: 56 - DNA, 2791 bp, Bernardia pulchella
gaattcggcacgaggatactctctgacggagctaacatcggatcggatcataaagttatcgtctctcattctcagatctctcgcttactttttctcttaatccaaaattcaagaaaagtgaaaaatggagccgaaattgttgcacggtactttacatgctacaatctatgaggttgataagcttcatggagaaggtgggcacttctttcgcaagctcgtggaaaatattgaagagacagttggttttggcaaaggtgtcactaaactttatgcaactattgacttggagaaggccagagttgggagaaccagaatactggaaaatgaacaatctaatcccaggtggtatgagtctttccacatttactgtgctcatgaggcttcaaatgtcatattcacagtgaaggatgacaaccctattgggacaactgtaattggccgggcacatttaccggtggatgagatcataaatggagaagaggtagataggtgggttgagatattggatgaacagggagaacctcttcgtcatggttccaaaatccatgtcaaactgcaatattttggagttgataaggaccgtaactgggggcgaggtatctggagtccaaaatatcctggagtaccttatacattcttctcacagagacaaggatgcaaagtttctctataccaagatgttcatataccagataaatttgttcctaaaattcctcttgctgggggcaagtactacgagcctcatagatgctgggaagatgtttttgatgcaattaccaatgcaaaacacttgatctacatcactggatggtctgtttatgctgaaatagccttgataagagactcgaggaggccaaagcctggaggagacatcactcttggtgagctgcttaaaaagaaggcaagtgaaggtgttagagtcctcatgctggtgtgggatgatagaacctctgttggattattgaaaaaggatggacttatggccactcatgatgaggaaactgaacattacttccagaacactgacgtgcattgtgttctgtgtcctcgaaatcctgatgatggtggaagctttgttcaggatcttcaaatatctactatgttcactcatcaccagaagattgttgtggtggacagtgcattgcctagtggagatcccgagagaaggagaattgtgagctttgttgggggtattgatctctgtgatgggagatatgatactccattccattcccttttcaggactctggacacatcacatcatgatgatttccatcagcccaattttgctggtgcttcaattgaaaaaggtggcccaagagaaccttggcatgacattcactccagacttgaaggacctattgcctgggatgtcttgtttaattttgagcagagatggagaaaacaaggtggtaaagatttgcttgttcagctgagagagctggaagatgtcatcatccctccatctcctgttttgtaccctgatgactttgaggcatggaacgtccagttgtttagatcaattgatggtggggctgcatttggttttcctgagacacctgaagatgctaccagagctgggcttgtcagtggaaaggataacattatagatagaagtattcaggatgcttatatcaatgccattcgaagggcaaagaatttcatttacattgaaaatcagtatttccttggaagttcttttggttgggctcctgatggtattaagcctgaggatattaatgcgctgcatctgattcccaaggaactttcactcaaaatagtcagcaagattaaggcaggggagagatttacagtttatgttgttgtccctatgtggcctgagggtatcccggagagtggatcagttcaggctatattagattggcagaagaggacaatggaaatgatgtataaagacattatcaaggcacttaaagaagagggcattatggcggatcctcggaattacttgacatttttctgccttggtaaccgtgaagtcaaaaaaagtggcgaatatgagccttcagaaaaaccagagcctgattcagattatataagagcacagcaagcccggcgtttcatgatttatgttcatacaaagatgatgatcgtggatgatgagtacataataattgggtctgccaacatcaaccagagatcaatggatggtgccagggactcggaaatagccatgggagggtaccaaccacatcacttgtcaaccaggcagccagcacgaggtcagattcatggtttccgcatgtccctatggtacgaacacctgggcatgctggatgattcgttcctcattccacaagatgaagaatgcgtcaggaaggtgaaccagatggcagacaaatattgggacctttattcaagcgagacgcttgaacatgacctacctggtcatttgctcaggtaccccattggtgttgctagtgaaggagatgtcacagagctccctggaatggagttcttccccgacacaaaggctcgagttctcggtgccaaatctgattacctccctcctatccttacaacttaattccaactccatgcgacagtttttctaataattacctatgttgccatccagtttatgttaccagttagcctaaaaataaatcatgttttacccagttctgcctgtattgtttttatgccagggtctatcacgatttacagatgtcataatgctttggtgtgctgtgatactgactgcgttgaacctttattttttttgacgttttgctatctgatccatgtatgtttttagagaaagaaaaaa

SEQ ID NO: 57 - DNA, 1718 bp, Bernardia pulchella
gcttgtacataacacacacggccgagtcatgtcgcaaacgaaacccgcacgaaactttccctcaatatcggaatgtaccggttcatcttacgaatccatagcggctgatctcgacggaacgttactcttgtcgagcagctcgtttccttactttatgctcgttgctgttgaagctggaagtttactgcgtggtttagtgttgcttttgtcgttaccattcattattatctcttattttttcatatctgaagcccttggaattcaaattctcatcttcatttctttcgccggtgttaagatccgcgatattgagctcgtttcccgcgctgttttgcctaggttttatgctgcggacgtgaggaaagatagttatgaggtgttggataggtgcaagaggaaagtggtggtgacggcaaatccaacgatcatggttgagccatttgtgaaggattttcttggaggagataaagttttaggcacagagattgaagtaaaccctaaaacaaagagagctacgggatttgtgaagaagccaggcgtgttagtgggaaaatggaagaaattagccgttatgaaagagttcggagatgaatcacctgatcttggtattggtgatcgtaaaaccgatcatgatttcatgtcgatttgcaaggagggttatatggttcgtcgcaccaaatcagcagtgtcactaccacgagaacagctgaaaagccgtataatcttccacgacggtcgtttcgttcaacgaccagacccactgaatgctctcgtcacatacatttggttaccatttgaattcatcctctccatcattcgcgtgtacttcaatctcccactccctgaacgaatcgtacgttacacgtacgaattattaggcatccacctcatcatacgtgggaccccacctccacctccatcacgtgggacccaaggaaatctctacgtgtgcaatcatcgcacagcgctggacccaatagtcatagccatagcacttggacgtaaagtctcatgcgtcacctacagtgtaagtcgtctctctagatttctatctccaatcccagccgtggctttaacacgtgatcgtgcggctgatgcagcacgaattacgtctttacttcaaaagggtgatcttgttgtgtgtcctgaaggaacgacatgccgtgaggagtttttgttgaggtttagtgctttgtttgctgaaatgagtgatagaattgtgcctgtggctgtgaattgcaagcaaagtatgttttatgggacaacagtacgtggggttaaattctgggacccatattggttttttatgaacccaagaccaacatatgaagtaaggtttcttgatcgtttgcctgaagagatgacggtaaaggccggtggaaaatcgtcgatcgaggttgcgaattatgtgcaaaaagttttgggtgatgtgttgggatttgagtgtactgggttaactaggaaagacaagtacttgttactaggcggaaatgatgggaaagtggagtccatgcatacctccaaaagtaagtgagattttttttctaattattattattattagtatgttgggtttcaaaaacggatcattgttagttccattgcgcttcgtttggagatgttttggtgaatgtatgactatatgtataccttgtaatttggagatttttagaattgaaacatcatattatttaactcttaaaaaaaaaaaaaaaaaa

SEQ ID NO: 58 - DNA, 1553 bp, Bernardia pulchella
gttctccgtcgcaatggcatcacccaactcggtaaaaatactcgaggtccaccacgtttctccggcaaccgtctcagctgagtcatcaaccgagtcatcactcccactcactttctacgagtccaattggctcaaattgccaccgacacaaaaccttttcttctacaaactcactgactcaacttcctttcactcaaccattttccccacactcaaacactcactcgctcgaacgctcgaccacttccggccagtcgccggtcacctgacgtgggccacagatgaacccagacccatgattcagttttctccaaatcacggtgtttctctcacactagccgagtcgtcaaacactaacttcaaccagtatatcagtgctaaaatccatgaagcagaaacggcacgtcattttgtacctgagctgcatatttcagatacatatgcatggacaatgtcggtccaaataacgttgtttcctaacaaagggtattgcattggtgtaacaacgcaccatgcaatatttgacggcaaaagtgctttaatgtttcttcaagcgtgggcttatatcactaatcaagttataaacaatgcagatacattctgtttggctacaggactagctccatcctttgacaggacagttatcacagaccccggtgagtttgagtctctttacttaaaccactggttaaccattaataaattagaatcacgttcgaatcccaaatgtttaaaagtgtcgaattttttcttaggcataccaaatgatttcgtgcggtcatcattttatctcagtcctgaaaacattaagaaaatcaaagaaagggtagttaaatttaagcctacacaacaatctaatgtatctacatttgtgattacgtgcgcatatgcgttagtatgcatggtaagatcggttagggcacggaaccaaaaggttgcgtttgtatttgcagtggattgtcgaaatcggtctgtcataaacccgaaaattcctgaaaattatttcgggaatgcgctcattctacatgatgtgattgtagaagcagaagattttatgggagaaaacggagtcgcgaccgtcgcgaaaaaaattagcgagtatataaatggattagaaaaaggactgctagatggtataaaagacagaatggaaaggttgtctagagtgggagatgatttgttgaaatttggtatagctggaggaacaaggctggcgttttataaaatggattttggatggggaaatcctgtgaaggttgagattccttcgatcaatgtgaatgctctttctataatggaaggtagagatggccatggagttgagattggtttgggtttaaagaagcatgaaatggatgagtttgcttcattatttgctcaaggattgaactaaactaaatgtgtttgtgtgtatgtaactcttttttaataattcctttttaatatgaatgtgtgatgcaataataaagacgatttgagttttttttaaaaaaaaaaaaaaaaaaaaaaaaaaaaaaaaaaaaaaaaaaaaaaaaaaaaaaaaaaaaaaaaaa

SEQ ID NO: 59 - DNA, 1646 bp, Bernardia pulchella
ggaaatacacacaaatctctccctctctctctctctctctctcttcattttctgcttaattcaattttttttacgtttagtaaatcacttaaaccgccatggactccggcggcgagattcggattcgtcaaacacgacgactacccgattttttacagagcgtgaatctcaagtacgttaaacttggttatcattatctaatctctaatttattaaccttatgttttattcctttaattataatcacttcaattgaagcatctcaaatgaatctcgacgatcttcgtcatctatggctacaccttcagtataatctagtcagcataatcatttgctccgcttttctcgtggtcggtttgaccgtttacattatgacccgacccagacccgtttatttagtcgattattcttgctatcgtgctcctgatgctcttaaagctccatttgaccgattcatggagcactctaaactcaccggcgatttcgacgagtcatcgctcgagtttcaaaggaagattctggaacgctctggactcggtgaagaaacttacgtgcctgaagctatgcattatattcctcctcggccgtcgatggcggcggctagggaagaggcggagcaggttatgtttggtgcgttggataatttgtttgctaatacgaatgtaaaccctaaaaatattgggattcttattgttaattgtagtttgtttaatcctacgccttcactctctgctatgattgttaataagtataaattgaggggtaatattaggagttttaatttaggaggtatgggttgtagtgctggagttatatcccttgatttagctaaagatttgttgcaagttcataggaatacttatgctgttgttgttagtactgagaatattacgcagaattggtattttggtaataagaaatctatgttgatacccaattgtttgtttcgtgtcggtggtgctgctgttttgctttcgaataggtctagggatagaaggcgagctaagtataggcttgttcacgttgtgaggactcatcgtggtgctgatgataaagcatttagatgtgtttatcaagaacaagatgatgttgggaaaactggtgtttctttgtctaaagatttgatggctattgctggtgaagctttaaaagctaatattactacattgggtcctttagtcttaccaataagcgagcagcttttgttctttgctacccttgttgtaaagaagctgtttaataaaaagatgaagccttatataccggattttaagttggcgtttgatcatttttgtattcatgctggagggagagctgtgattgatgaacttgaaaagaatcttcagcttctacctgcacatgtagaagcatttaggatgacacttcataggtttggaaatacttcatcaagctcgatttggtatgaattggcttacgttgaggccaagaggaggatgcgcaaaggtaaccgggtatggcagattgcttttggaagcggttttaaatgtaacagtgcagtgtgggaggcacttcaaaatgtcaaaccgtctcacagtggcccatgggaagattgtatagataagtatcctgtgacactatctgtttaattcaagaaataaatgcaattgaaaaaaaaaaaaaaaaaa

SEQ ID NO: 60 - DNA, 1569 bp, Bernardia pulchella
actactgtaactactgatcttcataaatctttttcgggtggatttcgggtcggagattttctttccaaaaatggcagataatcataaaaatgaagtgaatattacatgcaaatctcacgtgaagcctaataggaagataggaagaaaagaatgtcagcttgtgacttttgatcttccatacttagcgttttactacaatcagaagctgttgttgtacaaaaagagtgacgaacatgtgtttgaagatgtagtggagaagcttaaagatgggttgagggtagttttggaagattttcatcagttggccggaaaattagggaaagacgaagagggtgtttttagagttgaatatgacgatgacatggaaggtgttgagataattgaagccgtcgctaatgatatcagtatcgacgatctcattgttgatgaaggtacaacttcttttaaagacttgattccttataatgggatcttgaatttagaagggcttcataggccattgttggcagttcagctaactaaacttaaagatggcatcgcaatgggctgcgggttcaaccacgccattttagacggtacgtccacgtggcatttcatgagttcatgggccgagatttgcagaggctcgacttcagtttccgttccaccatttatcgaacgcacgaaggcgcgtgacactcgagtgaagctcgatctcacgcttcccacagattcatcatccaacggtgaaccaaatcctgtcccacaactaagagaaaaagtctttaaattctccgaaactgcaatcgacaagatcaagtcaatggtcaacgtaaatcctccatccgacggttcaaaaccattctccacatttcaatctttagcagttcatatttggcgccacgtaaccaatgcacgtgaactcaaacctgaagacatcacagtcttcactgtcttcgccgactgccgaaaaagagtcgatccaccgatgccggaaagttacttcggaaacctgattcaagctattttcaccgcaacagctgccggattgctaacgatgcagccaccggagtttggtgcgtcggttatacaaaaggcgatagaatcacataacgcaaaagcgatcgacgaacgtaacaaagaatgggaatctgcaccgaagatttttcaatttaaggacgccggagttaactgcgtcgctgtcggaagttcgccgagatttccggtttatgatgtggatttcggatttggaaagcctgaaagtgtacggagtggaagcaataataggttcgatggaatggtgtatttgtatcaagggaagaatggaggtaagagtattgatgttgagatcagtttggaagctggagttatggagaaattggagaaagataaggatttccttatccagctttagaataacattaacatggaaagggttgagcttttgggagtgtttggatttgtgttgattttaatttgttttgtttatttttagtttgatgatgttggtatgatctgattcattgatatgaatgcagaaatcttaaaaataaacatataattgatttcatcttgtgaaaaaaaaaaaaa

SEQ ID NO: 61 - DNA, 1553 bp, Bernardia pulchella
aagctctctcctcggtttcacgtgaagaaaaaaatggacaaagcaatcaataggcaacaaattattctagatcatctccgtccttcttcatcttcacataattacgagtcctctctctccgcatctgcttgcttagctggagatagcgccgcttatcaacggacttcggtttatggagacgatgttgtgattgtagcagcacatagaactgccttgtgcaaatccaagcgtggtggtttcaaggatacttatgccgatgatttacttgctcctgtattgaaggcattgatagagaaaacaaatttgaacccaagtgaagttggggatattgttgtgggaacagtattggcaccaggatctcaaagagcaagtgaatgtaggatggctgcattctatgctggttttcctgaaattgtgcctgtgaggactgttaacaggcagtgttcatctgggcttcaggcagttgctgatgtagctgctgctatcaaagcagggttttacgacattggtattggagctggattggaatccatgacaattaatccaatgtcttgggatggagatgtgaacccaaaagtaaaggcttttgagcaagcccaaaactgccttctgcccatgggtgttacttccgaaaatgttgctcatcgttttggagtgactagacaggagcaggatcaggctgcagttgagtctcatagaaaggctactgctgctactgctgctggcaaattcaaggatgaaattatcccagtgtctaccaagatcaaggacccaaaaactggtgaggagaaacatatcactgtctctgttgatgatgggtttcgccctaatgcatcactctctgatctagctaaacttaaacctgtgtttaagaaagatggaaccaccactgctggtaattctagccaggtcagtgatggtgctggggctgtgttgcttatgaagagaagtgttgccatgcgtaaaggtctccccgtacttggagtattcaggacttttgctgctgttggtgtggatcctgccatcatgggagttggcccagctgttgcaattcctgctgcagtgaaggctgccggtctagaacttaaggacattgatctttttgagataaatgaggcctttgcttcccaatttgtttattgcagaaagaagttagagctagatcctgagaagatcaatgttaacgggggtgcaatggccattggacatcctctgggtgcaacaggtgcccggtgtgttgcaactctattgcatgagatgaaacgcagaggaagagactgtcgttttggagtagtgtcaatgtgcataggcacaggaatgggagcagcagctgttttcgaaagaggagatgcatgtgacgatctctgcaatgcccgcaaagtagaagccaataatttgttatcaaaggatgctatttaaaccaaggccttgtattggttattgtagaacttgaaatatataatggcacattgcgatggcaaggagttgccacttctgaaaatgaaaatgttggtgtcaaaaaaaaaaaaaaaaaaaaaaaaaaaaaaaaaaaa

SEQ ID NO: 62 - DNA, 1870 bp, Bernardia pulchella
cccattctctctttttcttccatttcttcatcacacttcacaaaatgtcaacacaaaactcaaatcataatctgcccaattttcttctatccgttaagctgaaatacgtaaaattaggttaccattatctcatcactaacgctatgtatcttttactcgttccagttttagcaattttttcagctcatctctcaacactcacagtttcagattttgttcagttatggaaccaattaaagttcaatttcgtatctgtcactgtatgttccggtctcatggtctttctatcgacgctttacttcacaagccgtccgagaaaaatatacttggttaatttctcttgttacaagcctgaagattctagaatatgcacgagagaaatgtttatggagaggtcgaaattggcgggaaaatttacagaagagaatttgaattttcaaaagaagattctagaaaggtctggattaggtcaaaagacttatttacctgaagctgttatgagggtcccaccgaatccttgtatggctgaagcgaggaaagaagctgagatggttgtgtttagtgccattgatgagttgttagagaaaactggcgttaaagctaaggatattgggattcttgtagtgaattgcagtttgttcaatccgacgccgtctttgtctgcaatgataatcaatcattacaagctcagagggaatattttgagctataatcttggtggtatgggttgcagtgctggacttatttccattgatctcgctaaacagctcttacaggttcagccaaactcctatgctcttgtagtcagcatggaaaacattacactaaactggtattttggaaatgaccgatcaatgctcgtctcaaattgcctattccgaatgggaggagcagcgattcttctttcaaacaaatcatcggatcgccgtcgatcaaagtaccaattgatccacaccgttcgtacccacaaaggctccgatgataaatgttacaactgtgtattccaaaaagaagaccaaagtgaacaaaaaacaatcggtgtctcattatctaaagacctcatggccgtcgcaggagaagctctcaaaacaaacataaccacactaggcccattagtcttaccaatgtctgaacaattattgttttttgcaacattagttgcaagaaaagttttcaaattgaaaatcaaaccatacattcctgattttaagttagcttttgaacatttttgcattcatgctggtggaagagctgtgttggatgaattagagaagaatctggaactttctgagtggcacatggaaccttctagaatgacactttatagatttggtaatacttctagcagttctttgtggtatgagttggcgtactctgaagctaaaggaagaattagaaaaggtgataggacgtggcagattgcgtttggttcagggtttaagtgtaacagcgctgtttggaaagcattgaagaatattaatccggataacgagaagaatccatggatcgatgaaattgatgaatttccggttcatgtgccaagattagtctcaatttttacttcgagttgattgaaccattagcatttggtttagtaaaacatgggtgagttttatttattaattttgttttgttgattgttggtggaagcctcttgggtaggtcaaaacatacaagaggaatacaaattcaattatcttttaccagaatttgtttgataattttttttctatgtattcttataaattgtacatctttgggatatgatttacttccttttaagggattaaatgtattgttattaatagttttgaattataaacataccaaaaataacacacattcgtaataaaaaaaaaaaaaaaaaaaa

SEQ ID NO: 63 - DNA, 637 bp, Bernardia pulchella
acggacacgtcagcagcaacattggagtgggctatgtcgcttttggtaatgcaccctgaagttctgagaaaggctagagcagagctagacagagtggttggtgaagagcgattagtagaggagtctgattattcgaagctgccttatttgaaaaacatcatagacgaaacgtttcggttgtacccaacagcaccactgttagtgccccatgaatcatcggatgattgcgttattgggggatataatgtacgcaaaggaaccatgttgctggtcaatgcgtgggctattcacagagacccaacgctgtggaaagaaccaacaagttttagacctgagagatttgacggtgaagaaactgacacgtataagttgattccttttgggattgggaggagatcctgtcctggtgctgggcttgcgattaaactggtgagcgtgacgttggctgcattaattcagtgctttgaatgggaaacagttggtgaacagataattgatatgaatgagggaacagggctcactatgccgaaagctcagccattggaagccatgtgcagagcccgtgagtcaatgattaacgtcccttgaaaagttgtaggtaattgatgtttgactgcgtgtttcccgaaataataat

SEQ ID NO: 64 - DNA, 1492 bp, Bernardia pulchella
cttcactctccttcctcatttatacaaaatacagagaatttatacaaaatacagagagagagagaattcttaagggaaaggaaactctttccttcaatacctcaggttgtggaacgatgggtgctggtggcagaatgtctgttcctccttcacctaagaatgttgaatctgacatcttaaagcgagttcctcattcaaagccgccattcgccctcggtcagataaaaaaagctatcccacctcactgtttcaaacgatctattccgcactcgttctcatatgtagtccaggatctgaccattgcgtttctcttctactacattgctaccaattacttccacctcctccctcatcctctctcttttgttgcctggccaatttactgggctgtccaaggctgtgttctcacaggtgtttgggttatagcacatgaatgtggtcatcatgccttcagtgactatcagttgcttgatgatattgttggccttgtcctccattcttgccttcttgtaccttacttctcatggaaacacagccatcgccgtcaccattcaaacacagggtccttggaacgtgatgaagtattcgttcccaagcagaaatccagcatccgttggtattctaaatatcttaacaacccattcggtcgtgtcttaacactcactgttacactcactcttggatggcctctatacctagcgttcaatgtttcaggcaggccatatgatcggtttgcctgccactatgacccatacggtcctatctacaatgaccgtgaacgaattgagatattcatttctgatgttggtattcttgctgtcacttatggtctctaccgactcgccgtagccaagagtcttgcttgggttatttgcgtctatggagtacctctattggtagtgaatgcgttccttgttctaatcacattcttgcagcacactcatccttcattaccacactacgattcgtctgagtgggattggctaagaggagctctagcaactgttgacagagactatggaatcttgaacaaagtgttccataacataacagatactcatgtagctcatcatttgttttcaaccatgccacattaccatgcaatggaggctacaaaagcaattaaaccaatcttaggagaatactaccaattcgacgggactccttttattaaggcgatgtggagagaggctaaggaatgtgtttatgtagagcaagatgacggtgatcagagcaagggcgtgttctggtataacaacaagttttgatcacattgctaatcaaatcttggaagctctggtgttgcttgttagaattctagcctttgttgttctcggtaatgtaggctcttcacctgtcatgaacagaacttcagtctctgtcttgctctttttttcctcttaacaactatttctgctcatgattgtctagctcagtgctcttctactactatgaatgcatcctatcgatcacaaaaaaaaaaaaaaaaaaa

SEQ ID NO: 65 - DNA, 1122 bp, Bernardia pulchella
aactacttccatcttctaccgtctccactctgctacattgcttggcctgtctattgggttttccagggttgtgttcttacaggggtttgggtcattgctcatgaatgtggtcaccatgcttttagtgactatcaattggttgatgatattgttggcctcattctccattctgcacttctagttccttacttttcatggaaaattagccaccgtcgccaccattcaaacacaggttctctcgatcgtgatgaagtgtttgttccaaaaccaaaatcaaaaattgcatggtactttaagtacttaaacaacccgcttggtagaatgttaacgcttgtaaccacacttattggtggttggcctttatacttgacccttaacgcctcgggcagacattacgatcgctttgcttgtcactatgatccctacagtccaatatattctgaaagagaaagggctctgatatgcatttctgatattgggatatttattacaagtttcgtgctttatcaagttttcatgttgaaagggttgagttgggtgatatgcatctatggagtaccattgcttatagttaacgcttttcttgttatgatcacatacttgcaacacactcaccctgcactaccacactacgattcatcagaatgggattggttaagaggcgcattgtcaacagccgacagagactacggagtgttgaacaaggtatttcacaacatcacagacactcatgtagctcatcatctcttctccacaatgccacattatcatgcaatggaggctaccaaagtcattaaaccgatattgggcgagtattatcggtttgatggtaccccaatttacaaggcattgtggagagaggcgaaagagtgcatgtttgtagagccggacgaaggaactcgcgacccaggtgtgttctggtataggaacaagttttaagacattgaatatgtaacattggcgtgtgtggtaataatggtgttgaagcatgtatttactagtacagtagccttcttacaaggaaggtgaatatgttaagtcaagtgtgttgatcaaactctttcaatattgtgataacgacagtttcgatttcttatcgtgtgcaaaaaaaaaaaaaaaaaa

SEQ ID NO: 66 - DNA, 1433 bp, Bernardia pulchella
gcaaaatacagagagagcgagagagagggataagaagagagagagagagagagttcttaagggaaaggaaacatctttctctttccttcaatacctcaggttgtggaacgatgggtgctggtggcagaatgtctgttcctccttcacctaagaaggttgaatctgacgtcttaaagcgagttcctcattcaaagccgccattcaccctcggtcagatcaaaaaagctatcccacctcactgtttcaaacgatctattccgcactcattctcatatgtagtccaggatctgaccattgcgtttctcttctactacattgctaccaattacttccacctcctccctcatcctctctcttttgttgcctggcccatttactgggctgtccaaggctgtgttctcacaggtgtttgggttatagcacatgaatgtggtcatcatgctttcagtgactatcagttgcttgatgatattgttggccttgtcctccattcttgccttcttgtaccttacttctcatggaaacacagccatcgccgtcaccattcaaacacagggtccttggaacgtgatgaagtattcgttcccaagcagaaatccagcatcctttggtattctaaatatcttaacaatccattcggtcgtgtcttaacactcactgttacactcactcttggatggcctctatacctagcgttcaatgtttcaggcaggccatatgatcggtttgcctgccactatgacccatacggtcctatctacaatgaccgtgaacgaatcgagatattcatttctgatgctggtgttcttgctgtcacttatggtctctaccgacttgccgtagccaagagtcttgcttgggttatttgcgtctatggagtacctctattggtagtgaatgcgttccttgttctaatcacattcttgcagcacactcatccttcattaccacactacgattcgtccgagtgggattggctaagaggagctctagcaactgttgacagagactatggaatcttgaacaaagtgttccataacataacagatactcatgtagctcatcatttgttttcaaccatgccacattaccatgcaatggaagctacaaaagcaattaaaccaatcttaggagaatactaccaattcgacgggactccttttattaaggcgatgtggagagaggctaaggaatgtgtttatgtagagcaagatgacggtgatcagagcaagggcgtgttctggtataacaacaagttttgatcacattgcaaatcaaatcttggaagctctggtgttgcttgtttgaattctatcctttgttgttctcggtaatgtaggctcttcacctgtcatgaactgaacttcagtctctgtcttgctcttttttttcctcttaataattatttctgccaaaaaaaaaaaaaaaaaaaa

SEQ ID NO: 67 - DNA, 423 bp, Bernardia pulchella
tttataaaatggattttggatgggggaatcctgtgaaggttgagattccttccatcaatgtgaatgctctttctataatggaaggtagagatggccatggagttgagatcggtttgggtttaatgaagcatgaaatggatgagtttacttcattatttgctcatggactaaactaaatatgtttgtgtatgtatggttccttttttttttcaagaatttttttttggaagtgtgatataataaggtttggatatttttttacggaaattataaatgtaaaagtgaaagtgtgatataataagagtttgtataaaattaaaaattgatataaaattttaaaagtaaagtttaaatgttaaagtttcacttttataaaaaaaaaaaaaaaaaaaaaaaaaaaaaaaaaaaaaaaaaaaaaaaaaa

SEQ ID NO: 68 - DNA, 465 bp, Bernardia pulchella
aattcggcacgagggaaaggttgtctagagtgggagatgatttgttgaaattcggtatagctggaggaacaaggctggcgttttataaaatggattttggatgggggaatcctgtgaaggttgagattccttccatcaatgtgaatgctctttctataatggaaggtagagatggccatggagttgagatcggtctgggtttaatgaagcatgaaatggatgagtttacttcattatttgctcaaggactaaactaaatatgtttgtgtatgtatgcttccttttctttctcaagaattttcttttggaagtgtgatataataagctttgcatattttcttactgaaattatacatgtaaaagtgaaagtgtgatataataagatttgtataaaattaaaattgatataaattttaatataaaatttaatgttgaatttcattttaaaaaaaaaaaaaaaaaaaa

SEQ ID NO: 69 - DNA, 315 bp, Bernardia pulchella
gtaaaatctgagtcgaagctctcctggcgtttcacttaacttccgacaaccaccaacgaatagaaagaaattaaaaatggacaaagcaatcaataggcagcaggttattctagatcatctccgtccttcttcatcttcacataatttcgagtcctctctctccgcatctgcttgcttagctggagatagcgccgcatatcaacggacttcggtttatggagacgatgttgtgattgtagcagcacatagaactgccatatgcaaatccaagcgtggcggtttcaaggacacttatgccgatgatctacttgctcc

SEQ ID NO: 70 - DNA, 597 bp, Bernardia pulchella
ataacacaaatctctccctctctctctctctctctctcttcattttctgcttaattcaattttttttacgtttagtaaatcacttaaaccgccatggactccggcggcgagattcggattcgtcaaacacgacgactacccgattttttacagagcgtgaatctcaagtacgttaaacttggttatcattatctaatctctaatttattaaccttatgttttattcctttaattataatcacttcaattgaagcatctcaaatgaatctcgacgatcttcgtcatctatggctacaccttcagtataatctagtcagcataatcatttgctccgcttttctcgtggtcggtttgaccgtttacattatgacccgacccagacccgtttatttagtcgattattcttgctatcgtgctcctgatgctcttaaagctccatttgaccgattcatggagcactctaaactcaccggcgatttcgacgagtcatcgctcgagtttcaaaggaagattctggaacgctctggactcggtgaagaaacttacgtgcctgaagctatgcattatattcctcctcggccgtcgatggcggcggct

SEQ ID NO: 71 - DNA, 604 bp, Bernardia pulchella
aatcggccgagttgtggttatatgcctcttactaaaacaagtcctggaagcagcgcggctttcacgataccattcctaaggcatatatggtcatggggtggtcttacaccagcatcaaggcaaaattttgcaaacctcctggcttctggttacagtgtgattgttattcctggtggtgtccaagagatgttttatatgaagcatggttctgagatcgttttcgtcaagtcaagaagaggatttgttcgactggcgatagagatgggtaaacctttggttccagttttttgcttcggtcagtccaacgcttacaggtggtggaaacctcaaggcaagacggtcctgagaattgctagagccatgaagttcacccctatacttttggggggcattttcagaggtcctttacccttgaggcatccaatgcatgttgtggtgggtaaaccgattgaggtcgagccaaatccacagcctaccgtagaagaggttgcagaagtgcacaaccagtttgtgacggcactgaaagatttgtttgagaggcataaagcacgggttggctatccagacctaactctagaaattttttgacaaaaaatgaatttga

SEQ ID NO: 72 - DNA, 1769 bp, Bernardia pulchella
gccaaaagccaatcaaatggcagcagcagcagcaaccactagatctagtccaaagttcttgatagttgatccaaagaagggaaagaagagagatatatttaagtatcttgttgaaaacgatgtcaagagtggaatgaattttttggatagttcagaggatggagttaagggtggtgcagctgttgatcatagatcgcttttgttggtttctatcatcataataaggattcttagtttcttggagatacccttgaagttgcttggctatattgttgatttttttctcaatttcatttctcaaaatggtggcttctttggcattttcatcaactttctccatggaaagttggtgataccaaagagaggaagtgataattttataagcacaatcggacaattggatggacgtatacacctgtataagactccaatcctatcagagcaagtagatgactccattgccactgataatagcaatattaaatctggacttggcaaccgatatctcatggatctttctattatggctgccaaattagcttatgaaaatgccaaagttgttcaaactgttgtcgaccgtcactggaagatgcattttgaggttttctacaactgctggaatgagaatcaaaaacaatacaacacccaagctttcattttctgcgacaagccaaaagatgcaaacttgatagttgtcagcttcagaggaacagagccattcaatgcacaagattggaacactgatttcgatttctcatggtatgagatcccaaaagttggaaaaatccatatcggatttcttgaaggtttaggtttaggaaacagaagtgatgctcgctccttcgaaactcacctccagaaacaacatcaggttcctccgggttggcattctgaaggcacagccattgaatgggccaaaaggagcgcatattatgccgtagcaataaaacttcaaagcttgttacaagaacataagaacgcaaaatttgtggttacagggcatagcttaggtggagctctggcaatattgtttccgtcaattcttgtaattcaggaggagacagagatgttacagaggttgctgaatatatacacattcggtcagcccagagttggagacgagaagcttggaaattttatggaatcacatttgaattatcctgtcacaagatatttcagggttgtttactgcaatgatttagtgcctagagtgccttttgatgacaagatctttgctttcaagcattttggaacttgtctttactatgatagccattacttcggtcgatttatggacgaggaaccaaacagaaacttcttcggattgagccatataattccaatgagaatgaatgcattatgggaaatactgcgaagttttatgatagggcacacacatggaccagaataccaggaaagttggttttgcactgtggccagggttgcaggattactgcttcctggtgtttctgctcatcttcctgttgattatgtcaattcggttcgccttggaaaggagagagtgcctccattagaatccttgaaaagcttcgctcgccagttataaagtagattcacaaggctcaagtccttgttataattttccaggacccatcttaattatatttctagctacatctctatccttattgttgtgcgttgttttccatgtcttcagaataatgataatgattccgtgttggatttcactgtttatttacacagatatttcaataatatatgttatctataaaaaaaaaaaaaaaaaa

SEQ ID NO: 73 - DNA, 674 bp, Bernardia pulchella
gttggtgataccaaagagaggaagtgataattttataagcacaatcggacaattggatggacgtatacacctgtataagactccaatcctatcagagcaagtagatgactccattgccgctgataatagcaatattaaatctggacttggtaaccgatatctcatggatctttctattatggctgccaaattagcttatgaaaatgccaaagttgttcaaactgttgtcgaccgtcactggaaggcaagcactagttgcatcttctcgttttagtaatttttcttttgatatatcatttttagcttctaatttaatcttattctcaacttctgatgtgaaaatacagatgaattttgaggttttctacaactgctggaatgagaatcaaaaacaatacaacactcaagctttcattttctgcgacaagccaaaagatgcaaacttgatagttgtcagcttcagaggaacagagccattcaatgcacaagattggaacactgatttcgatttctcatggtatgagatcccaaaagttggaaaaatccatataggatttcttgaagctttaggtttaggaaacagaagtgatgctcgctccttcgaaactcacctccagaaacaaccatcagggttcctccgggttggcaattctgaaggcacagccattgaatgg

SEQ ID NO: 74 - DNA, 918 bp, Bernardia pulchella
aaaagataagcttttctctttcatgccatttatacctataaatttggtaattctgcttgctcaaccagtgtcaaagatactagtgaattcactagcgggaaaaaagggtagtgatcatcaggaaccaaaagccatggctgcaactactgctgaaaacagtaagaatgagtctgacgttcctccaagcttcttgattgttgaccctaaaaagggtagaaaaagagatatagtgaagtactttgtgaaaaaagatgcgaagagtggaatgagtttcttggatagttcctcagaaggaatcaagggtagtgcagcaattgaccatcggtggatcttattggtttctattgtacttcgaaggattcttgcacttatcgcaacgcctttgaagtaccttggttatgtcattgatttctttctcaacttaatttctcagaacaatggactttctggcattttcactaatttcctccatggaaagttgagggtaccgcaaagaggatcagacaaatttctaagcacaatcggacaattagatgggcgaatagacttgtaccgaacagttatcttggcggagcaagtagataatagtattgctaacgatcccaatataagatcagagttgggaaatagatatctgatggatctttgtatcatggcagccaagcttgtttacgagaacgagaacgttgttacaaatattgtcaacaatcattggaagatgaactttgaggctttctacgactgctggaatccgggtcaaaacgagagcaacacccaagtgttcatgttcactgacaagcctaaagatgcaaacatgatagtgataagcttcagaggaacagaaccattcaacgcactagattggagtacagattttgatttctcgtggtattggatcgcaaaaattggaaggatcca

SEQ ID NO: 75 - DNA, 328 bp, Bernardia pulchella
ggatgctgctcctggtattctgcacatagtcctattgattatgttaattctgtcaggcttggaaaagagagagttgctcctttagcctccttgaaaagcttcactcgcaacttataagataatttgcatttccaatgcttcttcaacatcatggatcagtatatcactcaatgttgtaggtttcaccatcaataagtaatccagaataaattcagtgtattttcttttatcataaaagttcaagtagatgttcaactaaaaaaaaaaaaaaaaaaaaaaaaaaaaaaaaaaaaaagaaacaaaaaacacaaaaaaaaaaaaaaaaaaa

SEQ ID NO: 76 - DNA, 484 bp, Bernardia pulchella
gactcttgaaggctgccaaatgggtttttgatacagagtgtgaggttctaagggatttgattgagatgtatccggattacaaattgacatttgtaggacattctttaggggcaggattggtgtcactgatgacaatatatgctattcagaatcaagatagattggggaacattgagagaggcggatcagatgctttgcgatggctcctgctagatgcatgtcactgaatttggctgtgagatatgctgatgtcattaattctgttgtgcttcaggatgatttcctacctcgtacaaccactgctctggaagatgttttcaaatctcttttctgtttaccttgtctgctatgcctaatgtgcttgaaggacacttgcacttttgaagagaaaatgcttagagacccaagacgactttatgctccaggccgcctataccatatagttgagagaaagcccttcaggataggacgatttcctccaatt

SEQ ID NO: 77 - DNA, 646 bp, Bernardia pulchella
ccttctccgattcttcgatctcctgtttcttcgattcttcactctactctttctttgattcttcactctcctctttcttcgatcttcttattcttatttttttctccaagatcttagctattatttcaataggtatcaattaggaatcatcagaatcaacttagcaagtaagataattaacaccgatatatggatctgcattccaggggatactattctcaatatgttcgttcagagttatgcttatccatctagatgtggtctgaccaaggagtttatgttaaaattcatgttaatttgtagcttctgtgaacagggaaaggaattcaagatcaaacacaagcacataaatcatcatcttcctgtttatagtcatactcttgcgaaaatactggtggaatatgcttctgcagtgtacatgtctgatttgaatgagttgtttacatggacatgctcaagatgttgtgatatgactgaggggtgtgagattatagagctgattgttgatgttgagcactgcttacaggaatctcacgatatggtgtataaagttgtccaatttggctgtgttccttatgcttaacatcattcatggtaatgcttgggctttacaaataaagctgagagttagtaaataagcttcggt

SEQ ID NO: 78 - DNA, 1290 bp, Bernardia pulchella
gaactagaggttacatgtttagagaattgaaaacgatgccgtttcattcgtccaaggctcccactttatttactcggtttcttgttatggttcacattcttgcatggactaataaaacaacagctttggttaagctgccggcgaacgtcacggcacctccggcggttatagtttttggagattcaatagttgatgcaggaaataataacaatatcaaaactcttataaagtgcaacttccctccttatggacttcatttctacggtggaatccccaccggaagatttagcgacggcaaaattccatctgacattatagcggaagaattgggaataaaagctactctgccagcttatttggacccaaatttgttgcctcaggatctcataacaggtgtgacctttgcttcgggtggttgcggatatgatcctattacaccaaaattagtgtcagttatatcgctagatgatcaattgaatcacttcaatgactacaaagagaaggtgaaagctattgttggagaagaacgagcaaatttcattataaccaatagtctatttctagtagtagcgggcagcgatgacatagccaacacttattttgatctgcgtgctagaaaagcacaatacgatgttcctgcatacactgatcttatggttgattcagcttctacctttgttcagaatttatataaaatgggagcaagaagggttggtgtatttggtgcaccaccaatcggatgtgtaccatcacaaagaacattggcaggggggcctaaaagagaatgtgcaagcaattacaatgaagcagccattctattcaactccaaattggccacacagcttgaatcccttacagccactctgcctcaaagtaaaattgtttacgtagacatctaccaacccctcctcgatttcatccaaaatcctcaacaatatggatttgaagttgcagataaaggatgttgtggaacaggagttttagaagttgcagtattatgtaatcaagtgacaccagtaacatgtgctaatgtttttaatcatttattttgggatagttatcatcctactgaaagagcttacaacattcttatcacacaacttcttgttaagtatgttcagaaatttttctgaagattggaatcttgatcttgttatatttcatcttcctttgtttaaattcatttatcttgtattactcgatgttttgttattctggagagagaaaaaatgcgaataataagaaccagagtctttaatcaaaaaaaaaaaaaaaaaaaaaaaaaaaaaa

SEQ ID NO: 79 - DNA, 842 bp, Bernardia pulchella
tatactgcatcaccatcatctcaaagttcaagagtatcccgagcgatttctcttgcaaaaaatgacgcgcacaaaacaggatggactgtatacttgttatcctggatactgtttcctttgcgttttatgttgttgctgcctttccatctttgtggtttgttttacaagaggaggtcaactgccccatcgatgagaagaagccataagcctttgcgagtacattctatcaggagaatatacaatgtgaaggataatgtcattcatcgcaccacggacaggagacgtggagttattgaggatcttcatctggcaattgagatagtcatagaagctatatttggttattttcataaagcggcacatttttttctttcgccgtcagaagctttcagagttgtacataaatggttttcatctcaaagcagttacaatgaagaaattcagaatggtgcctatgatgcatctgtccctactgcgactcttggagaaaatgatcctgctattacagaaaagaacactacttttaatcacttgttaaatacagatgcacgaacttgtcaggatgtcataacagaacttgggtatccatatgaagttattcgggtgattacttctgatggatatgttcttcttcttgaaagaattcctagacgggattcacggaaagttgtttatcttcagcatgggatcttggattcgtccatgggttgggtatctaatgggattgttggctctccggcatttgcagcatatgatcaagggtttgatgtcttcttgggattttcggggtttagtttccaggaacccgttgacagaacatatcttcccgagatattggaatt

SEQ ID NO: 80 - DNA, 482 bp, Bernardia pulchella
ggcacgaggcacatccccacaccatgccgccggaatccacgccgcccaacttctggggtgatatgcctgaggatgaatattacgcctcacagggagtcattaattctcagtcctactttcaaacggtcaatggcaaattgttcacgcagagcttcataccgttggatcaaaaagtcaaagctacggtctacatgactcacggctatggatcagatactggttggttgtttcaaaagatctgcatcagttatgctacttggggctacgctgttttcgccgctgatctttttggtcacggtcgatccgacggtctacgttgctatatgggtgacatggagaaaatcgcagccacgtccttgtcattcttcaagcatgtccgatacagcgacccatataaagatttaccagcattcttatttggtgaatcaatgggtggcctagcaactatgctaatgtattttcagtcagaacccaacatgtgg

SEQ ID NO: 81 - DNA, 1222 bp, Bernardia pulchella
  misc_feature (369)..(369): n is a, c, g, or t
gttttgttgatgtcattcagcagcttgatcatcctttgtttaagtgggctcctgctggaatcagaacacaagagtggtatgaaaggaattcaaaaggtttagaaatctttttcaagagctggatgccatcaccagatgttacaatcaaaggtagtttgtttttctgccatggatatggtgatacttgcactttcttctttgaaggtgttgcgaggcgaattgctgcatcaggatatgttgtttatgctttggatcatcctggttttggcctctctcaaggattgcatggcttacattccaaagtttcgatttacttagcttgacaactgtcatcgaacagatttgcaaataattaaagtgagaccggangttaaaagcgactgccttgtcttcttacttgtggcagtcaatgggtggagctgttaccctaaaggttcatttaaagcaaccaaatgcatgggatggaatgattcttgttgctcctatgtgtagaattgcagaggacgtgaagccaccaccgccaattttgaaggcagtaaccattttgtctaggttcctgccaaaggcaaaacttgttcctcagaaagatctagaagaggtgtttattagagacttgaagacaagaaagatggctgatctcaatgtaattggttacaacggtcaaatgcgacttaaaactgccgtggaacttctcaaagctacagaggaaatcgaggctcaactggagaaggtttcgtctccattgttgatccttcatggagctgctgataaggtgacagaccctcaggtgagccagttcctgtatgagagagcttcccgtaaagataagacgataaaactttacgaagaaggttatcattgtatacttgaaggggaacctggtgacagaatttttggcatttttgatgatatgatttcatggcttgatttacggagctcgactctgtcgagaaaataattttcccatggtagcttcatttttttggtttttgcctttttagatgtgccaaaaaagtttcaagatttataatttgtatacaaaccctaatccatgttgaggatcatgggaaatatgaagcctcatatagcagaatagcagctaaagcagatgtgctttgatgcagcaattacagaggggaatcagaagtagtataataaaattcattattttgtttgagccttaaaaaaaaaaaaaaaaaaaaaaaaaaaaaaaaaaaaaaaa

SEQ ID NO: 82 - DNA, 637 bp, Bernardia pulchella
aatccccgactgtttttcacgcaccatcttcttcttcttcctcttcttctatcgtctcctcttctcttcttcttcttcttcttcttcttcttcttcttcttccattgccatgaacgacgccattctcacttcttcttcttccattgccatgaacgacgccattctcacttcttcttcttcttcttcttatttcggtttatatgctttaccttttggttgagagcttccccttttggttgttggtttgtttgttttgggttgacagttcctttggttcgagaatttgatagagatgataatttgaacaatgatgtgagtgaagagctctcagatgagaagacttccgttccatatgaaccagtttatagtgatgagcaagcaaggttaaatatttaggtgaggagggagggcagtaaccatttgtaatccatgagatctcttgattgctgatagatttatatgtcgaagactttgcaaaagttctatgatagagctctaacaattggactttaaggatatgcaaaattgtacttcaaggttttcgagatagaagtttctgtgaaagattaactcttgcatcaagattcaaagctatccgatcaagacaaccaaattgatcgattaatgacctttatgt

SEQ ID NO: 83 - DNA, 723 bp, Bernardia pulchella
  misc_feature (63)..(63): n is a, c, g, or t
ggctgctggagcagcagtggtatactgaatccgtcccttagcatggactctgttctctcacgnttacacatccgcgtgaaataaatgcagctaaccaacttctcaaccatacaaacaaaactattcaagccactactatatatattgtatcaccgtaaataccaccattacacacatacagtactgcccatttccccatttttgttttaattccgtaagttcttctggcatttagattatagcatggagagtgtgttgaagaaatggtgttgggtagtttgtgtggttctgtgtttggtgttggatgtaggaggtcaacaggtgccgtgttactttatctttggagattcattggtagataatgggaataataatcagcttcagtctctggctagagctaattacatgccgtacggaattgattttccaggtggtgctactggaagattttccaatggtaaaaccactgttgatgagattgctgtgcagcttggtttcacgaacttcattcctccgtatgcaactgcaaggggtcaacaaatacttggtggagtaaattatgcatctgcagctgctggaattagagaggaaacaggacagcaactgggaggtcgtattagtttccgtggtcaagtaagaaactaccagaacactgtttctcaatttgttaacctgcttggagatgaagattcagctgcaaattacttgaaacaatgcatattt

SEQ ID NO: 84 - DNA, 480 bp, Bernardia pulchella
gcacgaggagaggaggtaaacctctgtaacttcttcccttttaagccatgggtgattcaagatgtgcctctcgtataataagcttcacattgcttctctttgcatgttcatgcaatgcacaaaacttagcagactgtagatttgaagctatgtatcagttcggcgattctctttcggacactgggaactccattgttgaagttcctcaagcttatcattccagattaccttatggtatgaccattggccaagcaaccggtagaccttcagatggatatctcatgatcgactatttcgcactatcagctggtcttccactgattcaaccttttgaaaatccaaaatcaaacttcacttatggagcagatttttcagttgctggagtcacagctttgccttggcaaactttatccaaattaggtcttgcacttggatactcaaacagttctgtcccggtacaaattgaatggttcaaaaagg

SEQ ID NO: 85 - DNA, 587 bp, Bernardia pulchella
tcggcacgaggaatacaatcatcattcttcttcttcttctttcttcctttgtgtctctgcaatggcaaccaacaagagctttttcttcaaatatgttctctgttttgtcatctttttgttcttctctcaatcacattctatagactttaattttcctgctgttttcaattttggtgattcaaattctgatactggtaacctgattgctgctggttttgaaagtattaatcctccttatggtcaaaattactttcaaaaaccttctaagagatactgtgatggtcgtctcactattgatttcctcttggatgcaatggatctacctttcttaaatccatatttggagtcaattgggttaccaaattttcacagaggatgcaactttgcagcagcaggatctaagattcttccagcaactgcatcatcagtcagccctttttcatttggaatccaagtgaatcagttccttcgatttaggttccgagctcttcaattgctagcaaaaggtaagaaacttgagaggtatctcccagcagaagattatttccagaagggtctatacatactcgatattgggcagaacgatc

SEQ ID NO: 86 - DNA, 27 bp, Artificial Sequence
  Oligonucleotide primer
tcgggtaccgcttttcgaaatggcgat

SEQ ID NO: 87 - DNA, 33 bp, Artificial Sequence
  Oligonucleotide primer
ttggatatcgacgtcatgacatcgatccttttc

SEQ ID NO: 88 - DNA, 28 bp, Artificial Sequence
  Oligonucleotide primer
ttaggtaccagtgacagatatgcccctt

SEQ ID NO: 89 - DNA, 27 bp, Artificial Sequence
  Oligonucleotide primer
atggagctcacagcttcaggtcaatac

SEQ ID NO: 90 - DNA, 24 bp, Artificial Sequence
  Oligonucleotide primer
gattctagagagacccaatttgga

SEQ ID NO: 91 - DNA, 32 bp, Artificial Sequence
  Oligonucleotide primer
tttcccgggtcaggcttctttccgagtaatcc

SEQ ID NO: 92 - DNA, 31 bp, Artificial Sequence
  Oligonucleotide primer
tccgaattcaaaaaaacgggttttcgacacc

SEQ ID NO: 93 - DNA, 29 bp, Artificial Sequence
  Oligonucleotide primer
cgtctcgagaagaagataactgcttattc

SEQ ID NO: 94 - DNA, 27 bp, Artificial Sequence
  Oligonucleotide primer
ttggaattcacgcaagatacaaccatg

SEQ ID NO: 95 - DNA, 32 bp, Artificial Sequence
  Oligonucleotide primer
atcctcgagacaacattattcttcttttctgg

SEQ ID NO: 96 - DNA, 21 bp, Artificial Sequence
  Oligonucleotide primer
ggttaggtgaaaacaataatg

SEQ ID NO: 97 - DNA, 21 bp, Artificial Sequence
  Oligonucleotide primer
gtcaggccagtaaaatttcat

SEQ ID NO: 98 - protein, 379 aa, Bernardia pulchella
METELEQMNPEPSQPKPEPEPVHGDKDDRPLLKSDSNRMQTESIEELEKKFAAYVRNDVYGPMGRGELPLAEKVLLGLAMVTLVPIRTAFAMVILLFYYVICRICTLFASPNGEERDDFAHMGGWRRAVIVGFGRLLSRTMLFIFGFYWISESYRVPEETEHYQNERKDEESEKEAERPGAIISNHVSYLDILYHMSSSFPSFVAKRSVAKLPLVGLISKCLGCVYVQRESKSSDFKGVADVVVERVREAHQNSSAPTMILFPEGTTTNGDFLLPFKTGAFLAGAPVLPVILRYPYQRFSPAWDSISGARHVLFLLCQFINFLEVTWLPIYYPSQEEKDDPKLYACNVRRLIAREGNLVMSDIGLAEKRIYHTALNGLF

SEQ ID NO: 99 - protein, 390 aa, Bernardia pulchella
METELEQMNPEPSQPKPEPEPVHGDKDDRPLLKSDSNRMQTESIEELEKKFAAYVRNDVYGPMGRGELPLAEKVLLGLAMVTLVPIRTAFAMVILLFYYVICRICTLFASPNGEERDDFAHMGGWRRAVIVGFGRLLSRTMLFIFGFYWISESYRVPEETEHYQNERKDEESEKEAERPGAIISNHVSYLDILYHMSSSFPSFVAKRSVAKLPLVGLISKCLGCVYVQRESKSSDFKGVADVVVERVREAHQNSSAPTMILFPEGTTTNGDFLLPFKTGAFLAGAPVLPVILRYPYQRFSPAWDSISGARHVLFLLCQFINFLEVTWLPIYYPSQEEKDDPKLYACNVRRLIAREGNLVMSDIGLAEKRIYHTALNGNNRLPSVLHQKDD

SEQ ID NO: 100 - DNA, 1398 bp, Bernardia pulchella
ggtcgagagagagcacgcactcaatccctttctccttaattatgcagtactctctatagcatccaatttacatcgttaaatggaaacagagcttgagcaaatgaatcccgaaccatcccaacccaaacctgaaccggaaccggttcacggagataaagacgaccgaccgcttctcaaatccgattctaaccgaatgcagacggagagtatcgaagagctggaaaagaaattcgctgcttacgttcgcaatgatgtgtacggtccgatgggacgcggcgagttgcctttggcggagaaggttttgttaggtttagctatggtgacgcttgtaccgatacgaacagcatttgcgatggtgattttgttattttattacgtgatctgtcggatttgtacactctttgcatcaccgaatggtgaagagcgtgatgattttgcacatatgggagggtggagaagagcagtgatcgttggatttgggagattgttatcgagaaccatgcttttcatatttggattttattggattagtgagagttatagggttcccgaggaaactgagcactatcagaatgaaagaaaagatgaagaaagtgaaaaggaagcagaaagacctggagcaataatatctaatcatgtatcttatttagatatattgtatcacatgtcttcttctttcccaagttttgttgctaagagatcagtagctaagcttcctcttgttggtctcattagcaagtgtcttggatgtgtctatgttcagcgggagtcaaagtcatccgatttcaagggtgttgcagatgtcgtagttgaaagagttcgagaagctcatcaaaacagctctgctcctaccatgattctttttcctgaaggcaccactacaaatggagacttccttctgccattcaagacaggtgcattcctagcaggagctcctgtgcttccggtgattcttagatatccttatcaaagatttagtcctgcctgggactcaatatccggggcccgccatgtgctttttctcctctgtcaattcattaatttccttgaagttacgtggttacctatttactatccatcacaagaagagaaagatgacccaaagctttatgcttgtaacgttcgacgattgattgctcgtgagggtaatttagtcatgtcagacattggactagctgagaagcgaatataccatactgctctcaatggtttgttttgacaatgctaattcggatacgccatctatttatttcgtaatgtgattctattactaaaagaaaaaagatacaaaagttttgagaactatgtgcagaatgagtgttgttgaaattttgctagaaaatgtatctacctggatgtttagcattgttatagttattgcagaggtataccggcatt

SEQ ID NO: 101 - DNA, 1362 bp, Bernardia pulchella
acatcgttaaatggaaacagagcttgagcaaatgaatcccgaaccatcccaacccaaacctgaaccggaaccggttcacggagataaagacgaccgaccgcttctcaaatccgattctaaccgaatgcagacggagagtatcgaagagctggaaaagaaattcgctgcttacgttcgcaatgatgtgtacggtccgatgggacgcggcgagttgcctttggcggagaaggttttgttaggtttagctatggtgacgcttgtaccgatacgaacagcatttgcgatggtgattttgttattttattacgtgatctgtcggatttgtacactctttgcatcaccgaatggtgaagagcgtgatgattttgcacatatgggagggtggagaagagcagtgatcgttggatttgggagattgttatcgagaaccatgcttttcatatttggattttattggattagtgagagttatagggttcccgaggaaactgagcactatcagaatgaaagaaaagatgaagaaagtgaaaaggaagcagaaagacctggagcaataatatctaatcatgtatcttatttagatatattgtatcacatgtcttcttctttcccaagttttgttgctaagagatcagtagctaagcttcctcttgttggtctcattagcaagtgtcttggatgtgtctatgttcagcgggagtcaaagtcatccgatttcaagggtgttgcagatgtcgtagttgaaagagttcgagaagctcatcaaaacagctctgctcctaccatgattctttttcctgaaggcaccactacaaatggagacttccttctgccattcaagacaggtgcattcctagcaggagctcctgtgcttccggtgattcttagatatccttatcaaagatttagtcctgcctgggactcaatatccggggcccgccatgtgctttttctcctctgtcaattcattaatttccttgaagttacgtggttacctatttactatccatcacaagaagagaaagatgacccaaagctttatgcttgtaacgttcgacgattgattgctcgtgagggtaatttagtcatgtcagacattggactagctgagaagcgaatataccatactgctctcaatggtaataatagactgcctagtgttttgcatcagaaagacgattgataatttcatgtactccctctcaattgatatcgaatctcagttgagatgcttagtttaatttctcttggtattttactcatgaatgacagccagaaacataaggttgttttctccccttctattgttgtgccattattgtctcttgtaaatttctctaattggagttgcgagcatcaatt

SEQ ID NO: 102 - protein, 325 aa, Bernardia pulchella
MEKESNKKDEGIRVINTRDVYPTNAFHSVIALSLWLGSIYFNILLLFFSFLFLPFSKFLLVVGFLLVFVFVPINEDSKLGRRLCRYVTLQTCSHFPITLHVEDMNAFHADRAYVFGYEPHSVLPFGVSVLSELCGYMPLTKTKVLGSSAAFTIPFLRHIWSWGGLTPASRQNFANLLASGYSVIVIPGGVQEMFYMKHGSEIVFVKSRRGFVRLAIEMGKPLVPVFCFGQSKAYRWWKPQGKTVLRIARAMKFTPILLGGIFRGPLPLRHPMHVVVGKPIEVEQNPQPTVEEVAEVHNQFVTALKDLFERHKARVGYPDLTLEIF

SEQ ID NO: 103 - protein, 325 aa, Bernardia pulchella
MEKESNKKDEGIRVINTRDVYPTNAFHSVIALSLWLGSIYFNILLLFFSFLFLPFSKFLLVVGFLLVFVFVPINEDSKLGRRLCRYVTLQTCSHFPITLHVEDMNAFHADRAYVFGYEPHSVLPFGVSVLSELCGYMPLTKTKVLGSSAAFTIPFLRHIWSWGGLTPASRQNFANLLASGYSVIVIPGGVQEMFYMKHGSEIVFVKSRRGFVRLAIEMGKPLVPVFCFGQSKAYRWWKPQGKTVLKIARAMKFTPILLGGIFRGPLPLRHPMHVVVGKPIEVEQNPQPTVEEVAEVHNQFVTALKDLFERHKARVGYPDLTLEIF

SEQ ID NO: 104 - DNA, 1168 bp, Bernardia pulchella
aagaagatggagaaagagagtaacaagaaagacgaaggaattagagtgataaacacgagagatgtgtacccaacaaacgcatttcactcggtgatcgcgttgtccttgtggcttggttcaatctacttcaacatcttactgcttttcttttcttttcttttccttcctttctccaaattcctcctcgtcgttggatttcttttggtcttcgtctttgttccgatcaacgaggacagtaagttaggccgtcgcttgtgcaggtatgtaactcttcagacttgcagccattttccaataactctccatgtcgaggacatgaatgcttttcatgctgatcgtgcttacgtttttggttacgagccacattctgttcttccatttggagtatctgtattgtctgaactttgtggttatatgcctcttactaaaaccaaggtccttggaagcagcgcggctttcacgataccattcctaaggcatatatggtcatggggtggtcttacaccagcatcaaggcaaaattttgcaaacctcctggcttctggttacagtgtgattgttattcctggtggtgtccaagagatgttttatatgaagcatggttctgagatcgttttcgtcaagtcaagaagaggatttgttcgactggcgatagagatgggtaaacctttggttccagttttttgcttcggtcagtccaaggcttacaggtggtggaaacctcaaggcaagacggtcctgagaattgctagagccatgaagttcacccctatacttttggggggcattttcagaggtcctttacccttgaggcatccaatgcatgttgtggtgggtaaaccgattgaggtcgagcaaaatccacagcctaccgtagaagaggttgcagaagtgcacaaccagtttgtgacggcactgaaagatttgtttgagaggcataaagcacgggttggctatccagacctaactctagaaattttttgacaaaaaatgaatttgagctgttgtacctctctccttgtgttaatgggttaacaaacttgcacaacattcaccaactctttatttgttgcatcaatgtttctttttagttgtgagaacactgcaagaataggggatttgccactagccattggagcaacgttggtatggaaattttatttgttgt

SEQ ID NO: 105 - DNA, 1227 bp, Bernardia pulchella
gagaatcgtgtaaaaacagagagaagaagaagaagaagaggaggaagaagaagaagaagaagatggagaaagagagtaacaagaaagacgaaggaattagagtgataaacacgagagatgtgtacccaacaaacgcatttcactcggtgatcgcgttgtccttgtggcttggttcaatctacttcaacatcttactgcttttcttttcttttcttttccttcctttctccaaattcctcctcgtcgttggatttcttttggtcttcgtctttgttccgatcaacgaggacagtaagttaggccgtcgcttgtgcaggtatgtaactcttcagacttgcagtcattttccaataactctccatgtcgaggacatgaatgcttttcatgctgatcgtgcttacgtttttggttacgagccacattctgttcttccatttggagtatctgtattgtctgaactttgtggttatatgcctcttactaaaaccaaggtccttggaagcagcgcggctttcacgataccattcctaaggcatatatggtcatggggtggtcttacaccagcatcaaggcaaaattttgcaaacctcctggcttctggttatagtgtgattgttattcctggtggtgtccaggagatgttttatatgaagcatggttctgagatcgttttcgtcaagtcaagaagaggatttgttcgactggcgatagagatgggtaaacctttggttccagttttttgcttcggtcagtccaaggcttacaggtggtggaaacctcaaggcaagacggtcctgaaaattgctagagccatgaagttcacccctatacttttggggggcattttcagaggtcctttacccttgaggcatccaatgcatgttgtggtgggtaaaccgattgaggtcgagcaaaatccacagcctaccgtagaagaggttgcagaagtgcacaaccagtttgtgacggcactgaaagatttgtttgagaggcataaagcacgggttggctatccagacctaactctagaaattttttgacaaaaaatgaattagagctgttgtacctctctccttgtgttaatgggtaaacaaacttgcacaacattcaccaactctttatttgttgcatcaatatgtttctttttagttgtgagaacactgcaagaataggggatttgccactagccattggagcaacgttggtatggaaattttatttgttgtt

(19) United States
(12) Patent Application Publication
(10) Pub. No.: US 2011/0218348 A1
(43) Pub. Date: Sep. 8, 2011

(54) POLYPEPTIDES AND METHODS FOR PRODUCING TRIACYLGLYCEROLS COMPRISING MODIFIED FATTY ACIDS

(75) Inventors: Xue-Rong Zhou, Australian Capital Territory (AU); Surinder Pal Singh, Australian Capital Territory (AU); Allan Green, Australian Capital Territory (AU)

(73) Assignees: Commonwealth Scientific Industrial Research Organisation, Campbell, Australian Capital Territory (AU); Grains Research and Development Corporation, Barton, Australian Capital Territory (AU)

(21) Appl. No.: 12/989,405

(22) PCT Filed: Apr. 24, 2009
(86) PCT No.: PCT/AU2009/000517

§ 371 Date: May 16, 2011

Related U.S. Application Data
(60) Provisional application No. 61/125,438, filed on Apr. 25, 2008.

(51) Int. Cl. C07D 303/42

(52) U.S. Cl. 549/513; 549/562

(57) ABSTRACT

The present invention relates to methods of producing modified fatty acids comprising a functional group which is a hydroxyl group, an epoxy group, an acetylenic group or a conjugated double bond. For example, seeds, seedoil and methods of making seedoil are provided wherein at least 23% (mol %) of the fatty acid content of the seed or seedoil comprises the functional group. Also provided are novel polypeptides, and polynucleotides thereof, which can be used to produce the modified fatty acids, particularly in transgenic plants and cells suitable for fermentation.

### FIELD OF THE INVENTION

The present invention relates to methods of producing modified fatty acids comprising a functional group which is a hydroxyl group, an epoxy group, an acetylenic group or a conjugated double bond. For example, seeds, seedoil and methods of making seedoil are provided wherein at least 23% (mol %) of the fatty acid content of the seed or seedoil comprises the functional group. Also provided are novel polypeptides, and polynucleotides thereof, which can be used to produce the modified fatty acids, particularly in transgenic plants and cells suitable for fermentation.

### BACKGROUND OF INVENTION

Plant oils such as seed oils mostly contain varying proportions of a limited number of fatty acids which are either saturated (no carbon-carbon double bonds), monounsaturated (one carbon-carbon double bond in the acyl chain) or polyunsaturated (two or three double bonds) in the carbon chains of the fatty acids. These are present predominantly in seeds as triacylglycerides (TAGs) which have a glycerol backbone with fatty acids esterified to all three hydroxyl positions of the glycerol.

Plant cells such as cells of developing seed embryos synthesise fatty acid backbones and undertake the first desaturation in their plastids. Saturated and monounsaturated fatty acids are exported from the plastid and transferred to lipids in the ER membrane where they are available for further desaturation or modification. They are then removed from the membrane lipids and used for the assembly of TAGs, the principle component of seed storage oils.

### Biosynthetic Pathway of FA

The first part of fatty acid biosynthesis in plants occurs in the plastids. In a first step, acetyl CoA is carboxylated by acetyl CoA carboxylase (EC 6.4.1.2) to form malonyl-CoA. Fatty acids are formed from the malonyl CoA by repeated condensation to a growing acyl chain bound to acyl carrier protein (ACP) by the action of a fatty acid synthase complex, to form 16:0-ACP. This is then elongated to 18:0-ACP and desaturated to form 18:1-ACP which enters the cytosolic pool esterified to CoA. From there, the fatty acid may be incorporated into mono-, di-, or triglycerides. Further desaturations or other modifications occur after the acyl chain is transferred to phospholipid, in particular when esterified to phosphatidyl choline (PC).

There are a range of metabolic routes by which fatty acids that are modified on PC can be transferred to TAG, and a number of enzymes have been characterized that play roles in the flux of fatty acids between the PC, acyl-CoA and TAG pools. These are shown schematically in FIG. 1. These enzymes are also thought to be involved in the transit of unusual fatty acids into TAG. The enzyme acyl-CoA:lysophosphatidylcholine acyltransferase (LPCAT, EC 2.3.1.23) reversibly transfers fatty acids between the PC- and CoA-bound forms. Phospholipase A1 or A2 (PLA 1, PLA2) can also transfer acyl groups to the acyl-CoA pool by cleaving fatty acid from PC, yielding non-esterified fatty acid which may be esterified to CoA by the enzyme acyl-CoA synthetase (EC 6.2.1.3). Three enzymes carry out the successive acylations of the glycerol backbone to produce TAG in the so-called Kennedy pathway using acyl-CoA substrates, these are glycerol-3-phosphate acyltransferase (GPAT, EC 2.3.1.15), lysophosphatidic acid acyltransferase (LPAAT, EC 2.3.1.51) and diacylglycerol acyltransferase (DGAT, EC 2.3.1.20). DGAT acts after dephosphorylation of the phospholipid by phosphatidate phosphatase (EC 3.1.3.4).

At least eight genes encoding GPAT and five genes encoding LPAAT have been identified in Arabidopsis, although it is unclear which isoform is most important in TAG biosynthesis in seeds. Genes encoding LPAATs with some selectivity for less common fatty acid substrates such as erucic acid have been cloned and have been used to increase the accumulation of these fatty acids in transgenic crop species, although the increases were slight (Lassner et al., 1995; Knutzon et al., 1999).

There are also two known CoA-independent routes for the potential movement of modified fatty acids from PC directly to DAG and TAG. PC backbones could be converted into DAG molecules through removal of the phosphatidylcholine headgroups by choline phosphotransferase (CPT, EC 2.7.8.2). DAG formed in this way would be available for the synthesis of TAG by the action of DGAT. Fatty acids can also be incorporated into TAG by direct transfer from PC by the enzyme phospholipid: diacylglycerol acyltransferase (PDAT, EC 2.3.1.158), but the quantitative role of this enzyme in TAG biosynthesis may vary in different systems. PDAT has been postulated to play a major role in removing ricinoleic acid and vernolic acid from PC in developing castor bean and Crepis palaestina seeds, respectively (Dahlqvist et al., 2000; Banas et al., 2000).

### Unusual Fatty Acid Synthesis

Fatty acids synthesized in plants are not limited to the 5 or 6 fatty acids common to all plants, but many other, modified fatty acids (MFA) are displayed across the plant kingdom. Many MFA present in seedoils of non-food plants would be of considerable value as raw materials for industrial use if they could be produced cheaply and renewably in high-yielding oilseed crops. These include fatty acids with differences in chain length ie. greater than 18 carbons, or modification by other functional groups. Most recent attention has focussed on those C18 fatty acids that are modified at the Δ12 position either by the addition of hydroxyl or epoxy groups or by the formation of acetylenic (triple carbon-carbon) bonds or conjugated double bonds. When found naturally, such MFAs usually accumulate only in seedoils, not in other tissues of the plant or phospholipid membranes. Engineered plants could provide alternative, renewable sources to petrochemicals for MFAs if they could be produced in seeds and accumulated in sufficient proportions in triglycerides. This requires that seeds be genetically engineered to (a) synthesise the MFAs in high amounts, and (b) transfer the MFAs preferably to all three positions on TAG.

Synthesis of several MFAs has already been demonstrated in transgenic seeds through expression of genes encoding modifying enzymes which catalyse the conversion of common fatty acids to MFAs. These include fatty acids with very long chain length and high levels of polyunsaturation (e.g. EPA & DHA), and fatty acids with modifications at the Δ12 position such as epoxidation (vernolic acid), hydroxylation (ricinoleic acid), acetylenation (crepenynic acid) and conjugation (e.g. eleostearic acid). However, without exception the percentage of the MFA in the transgenic seedoil was observed to be much lower than the levels accumulating in the organisms where the fatty acid modifying gene was sourced (often 80-90% MFA). For example, the level of ricinoleic acid (12-hydroxy-octadec-cis-9-enoic acid; 12-OH 18:1Δ9) in transgenic tobacco (<1%) or Arabidopsis (up to 17%) expressing an exogenous Δ12-hydroxylase was much lower than in the native castor (Ricinus communis, up to 90% ricinoleic acid) from which the hydroxylase was obtained (van de Loo, 1995). Similarly, when an epoxygenase cloned from Crepis palaestina was expressed in transgenic Arabidopsis seeds, the seed oil accumulated up to 15% vernolic acid (12,13-epoxy-9-octadecenoic acid) compared to about 60% vernolic acid in C. palaestina (Lee et al., 1998). Similarly low levels of the MFA were observed after expression in transgenic seeds of an acetylenase from Crepis alpina (Lee et al., 1998), conjugases from Morordica charantia and Impatiens balsamina (Cahoon et al., 1999), a conjugase from Calendula officinalis (Qiu et al., 2001) and a bifunctional destaurase/conjugase from the tung tree Aleurites fordii (Dyer et al., 2002).

In view of the consistency of these data, it is clear that additional factors operate in the native plants that accumulate high levels of the MFAs. It is not known what these are. Several factors have been suggested, including inhibition of endogenous Δ12 desaturase activity by the modified fatty acid and therefore reduction in substrate levels (Zhou et al., 2006), the presence of TAG assembly genes with specificity for the MFAs, different subcellular localization and assembly of the modifying enzymes in the endoplasmic reticulum (ER) or other compartmentalization in the plant cells, greater stability of the enzymes in the native plants, or a requirement for an appropriate metabolic context for efficient synthesis of the MFA and removal into TAG (Dyer and Mullen, 2008). The failure to accumulate high levels of MFA in the transgenic plants may be due to poor ability of the recipient plant to remove the MFA from membrane lipids and transfer efficiently to TAG.

Some of these factors have been tested experimentally, with modest success. Product levels have been increased by using plants with genetic backgrounds optimised for substrate levels, for example using Arabidopsis lines having mutations in the FAD3 and FAE1 genes for increased levels of linoleic acid as a substrate for the FA modification enzyme. Enzymes encoded by FAD3 and FAE1 genes otherwise divert the substrate into other reaction pathways. Alternatively, product levels could be increased by expression of an additional exogenous Δ12 desaturase gene (Zhou et al., 2006). Lu et al. (2006) screened a cDNA library of genes from castor for genes which were able to boost hydroxyl fatty acid accumulation in seed oils of transgenic Arabidopsis and identified three genes which were able to provide modest increases in the level of product.

However, despite these attempts, MFA product levels remain below about 20% as a percentage of the total fatty acid in the seedoil when the heterologous genes were expressed in oilseed plants. There is therefore a need to raise the level of MFA in TAG in plants, particularly plants having commercially useful levels of oil in their seeds.

### SUMMARY OF THE INVENTION

The present inventors have identified methods of producing seed oil with at least 23% of the fatty acid content of the seedoil comprising a modified fatty acid.

Thus, in a first aspect the present invention provides a method of producing seedoil, comprising the steps of

i) obtaining a transgenic seed having one or more modified fatty acids in its seedoil, and

ii) processing the seed to extract the seedoil,

wherein the modified fatty acids comprise a functional group which is a hydroxyl group, an epoxy group, an acetylenic group or a conjugated double bond, and wherein at least 23% (mol %) of the fatty acid content of the seedoil comprises the functional group, and/or the molar ratio in the seedoil of the fatty acids with the functional group to fatty acids lacking the functional group is at least 23:77.

Preferably, the seed is from any Brassica sp., Gossypium hirsutum, Linum usitatissimum, Helianthus sp., Carthamus tinctorius, Glycine max, Zea mays or Arabidopsis thaliana. The seed may be from Crambe abyssinica, Camelina sativa, Cuphea sp, Vernonia galamensis, or tobacco (Nicotiana tabacum). Preferably, the Brassica species is Brassica napus, Brassica juncea, Brassica rapa, or Brassica carinata. More preferably, the seed is from Linum usitatissimum or Carthamus tinctorius. In an embodiment, the seed is not from Glycine max or Arabidopsis thaliana or both.

In an embodiment, the method further comprises harvesting the seed. In a further embodiment, processing the seed comprises crushing the seed and/or extracting the seedoil with an organic solvent. In yet another embodiment, the method comprises purifying the seedoil, such as by degumming the oil, or clarifying the oil to remove impurities or chemically treating the oil such as, for example, adjusting the pH of the oil. The method may further comprise a step of fractionating the oil to reduce the level of some lipid components or impurities.

Also provided is a transgenic seed comprising one or more modified fatty acids comprising a functional group which is a hydroxyl group, an epoxy group, an acetylenic group or a conjugated double bond, and wherein at least 23% (mol %) of the fatty acid content of the seedoil of the seed comprises the functional group, and/or the molar ratio in the seedoil of the fatty acids with the functional group to fatty acids lacking the functional group is at least 23:77.

In another aspect, the present invention provides a transgenic Carthamus tinctorius seed having vemolic acid and/or ricinoleic acid in its seedoil, wherein at least 17% (mol %) of the total fatty acid content of the seedoil is vernolic acid and/or ricinoleic acid, and wherein the seed comprises an exogenous polynucleotide encoding a fatty acid hydroxylase or a fatty acid expoxygenase.

In another aspect, the present invention provides a transgenic Gossypium hirsutum seed having vernolic acid and/or ricinoleic acid in its seedoil, wherein at least 17% (mol %) of the total fatty acid content of the seedoil is vernolic acid and/or ricinoleic acid, and wherein the seed comprises an exogenous polynucleotide encoding a fatty acid hydroxylase or a fatty acid expoxygenase.

In another aspect, the present invention provides a transgenic Brassica sp. seed having vemolic acid and/or ricinoleic acid in its seedoil, wherein at least 15% (mol %) of the total fatty acid content of the seedoil is vernolic acid and/or ricinoleic acid, and wherein the seed comprises an exogenous polynucleotide encoding a fatty acid hydroxylase or a fatty acid expoxygenase.

In another aspect, the present invention provides a transgenic Linum usitatissimum seed having vernolic acid and/or ricinoleic acid in its seedoil, wherein at least 15% (mol %) of the total fatty acid content of the seedoil is vernolic acid and/or ricinoleic acid, and wherein the seed comprises an exogenous polynucleotide encoding a fatty acid hydroxylase or a fatty acid expoxygenase.

The seed of the invention may be further defined by the features as described herein with respect to the methods of producing the seed or seedoil from the seed, and vice versa.

In another aspect, the present invention provides a transgenic plant which produces a seed of the invention.

In an embodiment, the seed comprises an exogenous polynucleotide encoding a Δ12 desaturase.

In a further embodiment, less than 4% (mol %) of the total fatty acid content of the seedoil is linolenic acid.

In a further embodiment, the fatty acids with the functional group are C14, C16, C18, C20, C22 or C24 fatty acids or a combination of any two or more thereof.

In another embodiment, the fatty acids with the functional group are predominantly C18 fatty acids.

In yet another embodiment, the C18 fatty acids are C18:1, C18:2 or a combination thereof.

In a preferred embodiment, the fatty acids with the functional group are 12,13-epoxy derivatives of C18:1, or 12-hydroxy derivatives of C18:1.

In a further preferred embodiment, i) the hydroxyl group is bonded to carbon-12 of an acyl chain, ii) the epoxy group or the acetylenic group is between carbons 12 and 13 of an acyl chain, or iii) the conjugated double bond is between carbons 11 and 12 of an acyl chain of the modified fatty acids.

Preferably, the transgenic seed comprises an exogenous polynucleotide encoding a fatty acid hydroxylase, fatty acid epoxygenase, fatty acid acetylenase or fatty acid conjugase.

Preferably, the transgenic seed comprises an exogenous polynucleotide encoding a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), or diacylglycerol:diacylglycerol acyltransferase (DDAT), or a combination of two or more thereof.

In one embodiment, the transgenic seed comprises one or more exogenous polynucleotides encoding DGAT, GPAT, LPAAT, LPCAT, PLA₂, CPT and PDAT.

In another embodiment, the transgenic seed comprises one or more exogenous polynucleotides encoding DGAT, GPAT, LPAAT, LPCAT, PLA₂ and PDAT.

In another embodiment, the transgenic seed comprises one or more exogenous polynucleotides encoding GPAT, LPAAT, DGAT2 and/or PDAT.

In another embodiment, the transgenic seed comprises one or more exogenous polynucleotides encoding GPAT and LPAAT.

In another embodiment, the transgenic seed comprises one or more exogenous polynucleotides encoding GPAT and DGAT2 and/or DGAT3.

In another embodiment, the transgenic seed comprises one or more exogenous polynucleotides encoding LPAAT and DGAT2 and/or DGAT3.

In another embodiment, the transgenic seed comprises one or more exogenous polynucleotides encoding GPAT, LPAAT and DGAT2 and/or DGAT3.

In another embodiment, the transgenic seed further comprises one or more exogenous polynucleotides encoding LPCAT and/or PLA2.

In the above embodiment, DGAT2 and/or DGAT3 can be replaced with DDAT.

In another embodiment, the transgenic seed further comprises an exogenous polynucleotide encoding a desaturase and/or an elongase.

In a further embodiment, the desaturase is a Δ12 desaturase.

Preferably, the transgenic seed further comprises an introduced mutation or an exogenous polynucleotide which down regulates the production and/or activity of an endogenous enzyme of the seed selected from DGAT, GPAT, LPAAT, LPCAT, PLA₂, PLC, PLD, CPT, PDAT, DDAT, a desaturase, or an elongase or a combination of two or more thereof.

In an embodiment, the desaturase is a Δ15 desaturase.

In further embodiment, the elongase is an elongase which elongates a C18 fatty acid.

Examples of exogenous polynucleotides which down regulates the production and/or activity of an endogenous enzyme include, but are not limited to, an antisense polynucleotide, a sense polynucleotide, a catalytic polynucleotide, a microRNA, a polynucleotide which encodes a polypeptide which binds the endogenous enzyme and a double stranded RNA.

Preferably, the double stranded RNA (dsRNA) molecule comprises an oligonucleotide which comprises at least 19 contiguous nucleotides of a polynucleotide encoding the endogenous enzyme, wherein the portion of the molecule that is double stranded is at least 19 basepairs in length and comprises said oligonucleotide.

In a further embodiment, the double stranded RNA is expressed from a single promoter, wherein the strands of the double stranded portion are linked by a single stranded portion.

Preferably, the exogenous polynucleotide which down regulates the production and/or activity of an endogenous enzyme does not significantly effect the production and/or activity of an enzyme encoded by a transgene in the seed.

Preferably, for each transgenic polypeptide produced by the seed, the level and/or activity of an orthologous endogenous polypeptide is down-regulated when compared to an isogenic non-trangenic seed.

In a further aspect, the present invention provides seedoil comprising one or more modified fatty acids comprising a functional group which is a hydroxyl group, an epoxy group, an acetylenic group or a conjugated double bond, wherein at least 23% (mol %) of the fatty acid content of the seedoil comprises the functional group, and/or the molar ratio in the seedoil of the fatty acids with the functional group to fatty acids lacking the functional group is at least 23:77.

Preferably, the seedoil is obtained from a transgenic seed.

Preferably, the seed is from Brassica sp., Gossypium hirsutum, Linum usitatissimum, Helianthus sp., Carthamus tinctorius, Glycine max, Zea mays or Arabidopsis thaliana. More preferably, the seed is from Linum usitatissimum or Carthamus tinctorius.

Also provided is a method of producing seed of the invention, comprising growing a plant of the invention and harvesting the seed.

In yet another aspect, the present invention provides a method of enhancing the production of one or more modified fatty acids in a plant tissue or organ, the method comprising expressing in the plant tissue or organ,

i) a first exogenous polynucleotide encoding a fatty acid hydroxylase, a fatty acid epoxygenase, a fatty acid acetylenase, a fatty acid conjugase or a combination of two or more thereof, and

ii) a second exogenous polynucleotide encoding a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), or diacylglycerol:diacylglycerol acyltransferase (DDAT), or a combination of two or more thereof,

wherein the modified fatty acids comprise a functional group which is a hydroxyl group, an epoxy group, an acetylenic group or a conjugated double bond, wherein production is enhanced such that the level of the modified fatty acids comprising the functional group in the oil of the tissue or organ is increased by at least 6% as a percentage of the total fatty acid content of the plant tissue or organ after extraction of the total fatty acids from the tissue or organ with chloroform/methanol, and wherein the at least 6% increase is relative to the level of the total fatty acids in a corresponding tissue or organ having the first exogenous polynucleotide but lacking the second exogenous polynucleotide.

Preferably, the plant tissue or organ is from Brassica sp., Gossypium hirsutum, Linum usitatissimum, Helianthus sp., Carthamus tinctorius, Glycine max, Zea mays or Arabidopsis thaliana. More preferably, the plant tissue or organ is from Linum usitatissimum or Carthamus tinctorius.

In another aspect, the present invention provides a method of producing a transgenic cell with enhanced ability to produce one or more modified fatty acids compared to an isogenic non-transgenic cell, the method comprising introducing into the cell,

i) a first exogenous polynucleotide encoding a fatty acid hydroxylase, a fatty acid epoxygenase, a fatty acid acetylenase, a fatty acid conjugase or a combination of two or more thereof,

ii) a second exogenous polynucleotide encoding diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), or diacylglycerol:diacylglycerol acyltransferase (DDAT), or a combination of two or more thereof, and

iii) analysing the cell, or progeny thereof, for enhanced ability to produce the modified fatty acids when compared to an isogenic non-transgenic cell,

wherein the modified fatty acids comprise a functional group which is a hydroxyl group, an epoxy group, an acetylenic group or a conjugated double bond, and wherein steps i) and ii) can be conducted simultaneously or sequentially in any order.

As the skilled person will appreciate, step i) can be performed before step ii) and vice versa. Furthermore, more than two exogenous polynucleotides may be provided encoding three of more of the defined enzymes. In addition, one or more of the exogenous polynucleotides may be present in the same contiguous polynucleotide molecule.

Preferably, the cell is a plant cell or a cell suitable for fermentation.

Preferably, the cell is a plant cell and the method further comprises generating a transgenic plant.

As the skilled person would appreciate, step iii) may comprise analysing a tissue, organ or organism comprising said cell or progeny thereof.

Preferably, the method further comprises selecting a transgenic cell which produces oil with at least 23% (mol %) of the fatty acid content of the oil comprising the functional group, and/or selecting a transgenic cell which produces oil with a molar ratio in the oil of the fatty acids with the functional group to fatty acids lacking the functional group is at least 23:77.

Also provided is a cell obtained using a method of the invention, or progeny thereof.

In a further aspect, the present invention provides a method of producing a transgenic plant with enhanced ability to produce one or more modified fatty acids when compared to an isogenic non-transgenic plant, the method comprising,

i) introducing a first exogenous polynucleotide encoding a fatty acid epoxygenase, a fatty acid hydroxylase, a fatty acid acetylenase, a fatty acid conjugase or a combination of two or more thereof, into a first plant cell,

ii) introducing a second exogenous polynucleotide encoding diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), or diacylglycerol:diacylglycerol acyltransferase (DDAT), or a combination of two or more thereof, into a second plant cell,

iii) producing a first plant comprising the first exogenous polynucleotide from the first plant cell,

iv) producing a second plant comprising the second exogenous polynucleotide from the second plant cell, and

v) crossing the first plant or progeny thereof with the second plant or progeny thereof to produce a plant comprising the first exogenous polynucleotide and second exogenous polynucleotide,

wherein the modified fatty acids comprise a functional group which is a hydroxyl group, an epoxy group, an acetylenic group or a conjugated double bond, and wherein steps i) and ii) can be conducted simultaneously or sequentially in either order and steps iii) and iv) can be conducted simultaneously or sequentially in either order.

Preferably, the method further comprises analysing the first plant, second plant, the plant produced from step v) and/or progeny thereof for enhanced ability to produce the modified fatty acids when compared to an isogenic non-transgenic plant.

Also provided is a plant obtained using a method of the invention, or progeny plant thereof.

In yet a further aspect, the present invention provides a method of producing oil comprising one or more modified fatty acids, the method comprising expressing in a transgenic cell,

i) a first exogenous polynucleotide encoding a fatty acid hydroxylase, a fatty acid epoxygenase, a fatty acid acetylenase, a fatty acid conjugase or a combination of two or more thereof, and

ii) a second exogenous polynucleotide encoding a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), diacylglycerol:diacylglycerol acyltransferase (DDAT), or a combination of two or more thereof,

wherein the modified fatty acids comprise a functional group which is a hydroxyl group, an epoxy group, an acetylenic group or a conjugated double bond.

Preferably, the cell is a plant cell or a cell suitable for fermentation.

Preferably, the method further comprises expressing in the transgenic cell a third exogenous polynucleotide which down-regulates the production and/or activity of an endogenous enzyme of the seed selected from GPAT, LPAAT, DGAT, LPCAT, PLA₂, PLC, PLD, CPT, PDAT, DDAT, a desaturase, or an elongase or a combination of two or more thereof.

In a further aspect, the present invention provides for the use of a first exogenous polynucleotide encoding a fatty acid hydroxylase, epoxygenase, acetylenase, conjugase or a combination of two or more thereof, and a second exogenous polynucleotide encoding a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT) or a combination of two or more thereof, for producing a transgenic cell with enhanced ability to produce one or more modified fatty acids when compared to an isogenic non-transgenic cell, wherein the modified fatty acids comprise a functional group which is a hydroxyl group, an epoxy group, an acetylenic group or a conjugated double bond.

In yet a further aspect, the present invention provides a eukaryotic cell comprising an exogenous polynucleotide encoding a polypeptide which is:

i) a polypeptide comprising amino acids having a sequence as set forth in any one of SEQ ID NOs:1 to 42, 98, 99, 102 or 103,

ii) a polypeptide comprising amino acids having a sequence which is at least 30% identical to any one or more of the sequences set forth in SEQ ID NOs: 1 to 42, 98, 99, 102 or 103, and/or

iii) a polypeptide which is a biologically active fragment of i) or ii).

Preferably, polypeptide is a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), diacylglycerol:diacylglycerol acyltransferase (DDAT), epoxygenase, acyltransferase and/or phospholipase.

Preferably, the cell is a plant cell or a cell suitable for fermentation.

In yet another aspect, the present invention provides a process for identifying a nucleic acid molecule involved in the synthesis of triacylglycerols comprising:

i) obtaining a nucleic acid molecule operably linked to a promoter, the nucleic acid molecule encoding a polypeptide comprising amino acids having a sequence that is at least 30% identical to any one or more of the sequences set forth in SEQ ID NOs: 1 to 3, 5 to 7, 10 to 16, 98, 99, 102 or 103,

ii) introducing the nucleic acid molecule into a cell or cell-free expression system in which the promoter is active,

iii) determining whether the production of triacylglycerols is modified relative to the cell or cell-free expression system before introduction of the nucleic acid, and

iv) optionally, selecting a nucleic acid molecule which modified the production of triacylglycerols.

Preferably, the triacylglycerols comprise modified fatty acids comprising a functional group which is an epoxy group, hydroxyl group, acetylenic group, conjugated double bond or a combination of two or more thereof.

Preferably, the nucleic acid encodes an enzyme with activity which is glycerol-3-phosphate acyltransferase (GPAT), 1-acyl-glycerol-3-phosphate acyltransferase (LPAAT), diacylglycerol acyltransferase (DGAT), phospholipase C (PLC), phospholipase D (PLD), CDP-choline diacylglycerol choline phosphotransferase (CPT) phoshatidylcholine diacylglycerol acyltransferase (PDAT), and diacylglycerol:diacylglycerol acyltransferase (DDAT).

In a further aspect, the present invention provides a process for identifying a nucleic acid molecule involved in the production of fatty acid-CoA comprising:

i) obtaining a nucleic acid molecule operably linked to a promoter, the nucleic acid molecule encoding a polypeptide comprising amino acids having a sequence that is at least 30% identical to any one or more of the sequences set forth in SEQ ID NOs: 4, 8 and 9,

ii) introducing the nucleic acid molecule into a cell or cell-free expression system in which the promoter is active,

iii) determining whether the production of fatty acid-CoA and/or triacylglycerols is enhanced relative to the cell or cell-free expression system before introduction of the nucleic acid, and

iv) optionally, selecting a nucleic acid molecule which enhances the production of fatty acid-CoA and/or triacylglycerols.

Preferably, the fatty acid-CoA and/or triacylglycerols comprise modified fatty acids comprising a functional group which is an epoxy group, hydroxyl group, acetylenic group, conjugated double bond or a combination of two or more thereof.

Preferably, the nucleic acid encodes an enzyme with activity selected from: acyl-CoA:lysophosphatidylcholine acyltransferase (LPCAT) and phospholipase A₂ (PLA₂).

In another aspect, the present invention provides a process for identifying a nucleic acid molecule involved in fatty acid modification comprising:

i) obtaining a nucleic acid molecule operably linked to a promoter, the nucleic acid molecule encoding a polypeptide comprising amino acids having a sequence that is at least 30% identical to any one or more of the sequences set forth in SEQ ID Nos:21 to 24,

ii) introducing the nucleic acid molecule into a cell or cell-free expression system in which the promoter is active,

iii) determining whether the fatty acid composition is modified relative to the cell or cell-free expression system before introduction of the nucleic acid, and

iv) optionally, selecting a nucleic acid molecule which modified the fatty acid composition.

Preferably, the fatty acids comprise a functional group which is an epoxy group, hydroxyl group, acetylenic group, conjugated double bond or a combination of two or more thereof.

Preferably, the nucleic acid encodes an enzyme with activity selected from: epoxygenase or Δ12 desaturase.

In yet a further aspect, the present invention provides a process for identifying a nucleic acid molecule encoding an acyltransferase or lipase comprising:

i) obtaining a nucleic acid molecule operably linked to a promoter, the nucleic acid molecule encoding a polypeptide comprising amino acids having a sequence that is at least 30% identical to any one or more of the sequences set forth in SEQ ID Nos:1 to 20, 25 to 42, 98, 99, 102 or 103,

ii) introducing the nucleic acid molecule into a cell or cell-free expression system in which the promoter is active,

iii) determining whether the fatty acid composition such as the ratio of fatty acid-CoA:fatty acid-PC:triacylglycerol is modified relative to the cell or cell-free expression system before introduction of the nucleic acid, and

iv) optionally, selecting a nucleic acid molecule which modifies the fatty acid composition.

In an embodiment, the lipase activity is phospholipase activity.

In another aspect, the present invention provides a substantially purified and/or recombinant polypeptide comprising amino acids having a sequence as provided in any one of SEQ ID NOs: 1 to 42, 98, 99, 102 or 103, a biologically active fragment thereof, or an amino acid sequence which is at least 30% identical to any one or more of SEQ ID NOs: 1 to 42, 98, 99, 102 or 103.

Preferably, the polypeptide is a diacylglycerol acyltransferase (DGAT), glycerol-3-phosphate acyltransferase (GPAT), 1-acyl-glycerol-3-phosphate acyltransferase (LPAAT), acyl-CoA:lysophosphatidylcholine acyltransferase (LPCAT), phospholipase A₂ (PLA₂), phospholipase C (PLC), phospholipase D (PLD), CDP-choline diacylglycerol choline phosphotransferase (CPT), phoshatidylcholine diacylglycerol acyltransferase (PDAT), diacylglycerol:diacylglycerol acyltransferase (DDAT), fatty acid epoxygenase, acyltransferase and/or phospholipase.

Preferably, the polypeptide has enhanced enzyme activity on a first esterified fatty acid substrate comprising one, two or three acyl chains each of which may be the same or different, wherein one, two or three of the acyl chains of the substrate comprise(s) a functional group which is an epoxy group, hydroxyl group, acetylenic group, conjugated double bond or a combination of two or more thereof, wherein the enhanced activity is relative to a second, corresponding esterified fatty acid substrate lacking said functional group.

Preferably, the first fatty acid substrate is an acyl-CoA substrate comprising the functional group, or a diacylglycerol substrate or a phosphatidylcholine diacylglycerol substrate comprising the functional group on an acyl chain esterified at the sn-2 position

In an embodiment, the polypeptide can be purified from Bernardia sp, particularly Bernardia pulchella.

The polypeptide may be a fusion protein further comprising at least one other polypeptide sequence. The at least one other polypeptide may be a polypeptide that enhances the stability of a polypeptide of the present invention, or a polypeptide that assists in the purification of the fusion protein.

In another aspect, the present invention provides an isolated and/or exogenous polynucleotide comprising:

i) a sequence of nucleotides selected from any one of SEQ ID NOs: 43 to 85, 100, 101, 104 or 105,

ii) a sequence of nucleotides encoding a polypeptide of the invention,

iii) a sequence of nucleotides which are at least 30% identical to the protein coding region of one or more of the sequences set forth in SEQ ID NOs: 43 to 85, 100, 101, 104 or 105, and/or

iv) a sequence which hybridises to any one of i) to iii) under stringent conditions.

Also provided is a chimeric vector comprising the polynucleotide of the invention. Preferably, the polynucleotide is operably linked to a promoter.

In another embodiment, the present invention provides a cell comprising the recombinant polypeptide of the invention, the exogenous polynucleotide of the invention and/or the vector of the invention.

The cell can be any type of cell, preferably, a plant, fungal, yeast, bacterial or animal cell.

Preferably, the cell does not naturally comprise the polypeptide, polynucleotide and/or vector.

In yet a further aspect, the present invention provides a method of producing a polypeptide of the invention, the method comprising expressing in a cell or cell free expression system the vector of the invention.

In an embodiment, the method further comprises isolating the polypeptide.

In another aspect, the present invention provides a transgenic non-human organism comprising a cell of the invention.

Preferably, the organism is a transgenic plant or an organism suitable for fermentation such as a yeast or fungus.

Also provided is a seed comprising a cell of the invention.

In yet another aspect, the present invention provides a method of producing seed, the method comprising,

a) growing a plant of the invention, and

b) harvesting the seed.

In yet another aspect, the present invention provides a method of producing oil containing modified fatty acids, the method comprising extracting oil from the method comprising extracting oil from the seed of the invention, the plant of the invention, the cell according of the invention, and/or the transgenic non-human organism of the invention.

In an embodiment, the cell is of an organism suitable for fermentation and the method further comprises exposing the cell to at least one fatty acid precursor.

In a further aspect, the present invention provides a fermentation process comprising the steps of:

i) providing a vessel containing a liquid composition comprising a cell of the invention, or an organism comprising said cell, which is suitable for fermentation, and constituents required for fermentation and fatty acid biosynthesis, and

ii) providing conditions conducive to the fermentation of the liquid composition contained in said vessel.

In another aspect, the present invention provides a method of producing a modified fatty acid, the method comprising contacting a fatty acid esterified to phosphatidyl choline, glycerol or CoA with the polypeptide of the invention.

In a further aspect, the present invention provides a method of producing a fatty acid-CoA, the method comprising contacting a fatty acid esterified to phosphatidyl choline with the polypeptide of the invention.

In another aspect, the present invention provides a method of performing an epoxygenase reaction, the method comprising contacting a fatty acid with the polypeptide of the invention.

In another aspect, the present invention provides a method of performing a desaturase reaction, the method comprising contacting a fatty acid with the polypeptide of the invention.

Preferably, the fatty acid esterified to CoA.

In another aspect, the present invention provides a method of performing an acyltransferase reaction, the method comprising contacting a fatty acid with the polypeptide of the invention.

In another aspect, the present invention provides a method of performing a phospholipase reaction, the method comprising contacting a fatty acid with the polypeptide of the invention.

In another aspect, the present invention provides oil, or fatty acid, produced by, or obtained from, the seed of the invention, the plant of the invention, the cell according of the invention, and/or the transgenic non-human organism of the invention.

In another aspect, the present invention provides an extract from the seed of the invention, the plant of the invention, the cell according of the invention, and/or the transgenic non-human organism of the invention, wherein said extract comprises an increased level of the modified fatty acids relative to a corresponding extract from an isogenic non-transgenic seed, plant, cell or transgenic non-human organism.

In another aspect, the present invention provides a substantially purified antibody, or fragment thereof, that specifically binds a polypeptide of the invention.

In another aspect, the present invention provides for the use of a seed of the invention, the plant of the invention, seedoil of the invention, the cell of the invention, the polypeptide of the invention, the polynucleotide of the invention, the vector of the invention, the transgenic non-human organism of the invention, oil of the invention, the fatty acid of the invention and/or the extract of the invention for the manufacture of an industrial product.

Also provided is a composition comprising a seed of the invention, the plant of the invention, seedoil of the invention, the cell of the invention, the polypeptide of the invention, the polynucleotide of the invention, the vector of the invention, the transgenic non-human organism of the invention, oil of the invention, the fatty acid of the invention, the extract of the invention and/or an antibody of the invention, and a suitable carrier.

In another aspect, the present invention provides a method of identifying a polynucleotide which, when present in a cell of a plant, enhances the production of one or more modified fatty acids when compared to an isogenic cell that lacks said polynucleotide, the method comprising

i) obtaining a first nucleotide sequence for at least a part of a gene present in the cell which encodes a polypeptide involved in the synthesis of triacylglycerols,

ii) comparing the first nucleotide sequence with a second nucleotide sequence to identify a region which is not conserved between the first and second nucleotide sequences,

iii) designing a candidate polynucleotide to down-regulate the level of activity of the polypeptide in the cell,

iv) determining the ability of the candidate polynucleotide to down-regulate the level of activity of the polypeptide in the cell, and

v) selecting a polynucleotide which down-regulates the level of activity of the polypeptide in the cell,

wherein the second nucleotide sequence is from a different plant species but encodes a polypeptide with similar function to the gene.

In an embodiment, step ii) comprises comparing the 3′ untranslated region of the first and second nucleotide sequences,

Preferably, the gene is from Brassica sp., Gossypium hirsutum, Linum usitatissimum, Helianthus sp., Carthamus tinctorius, Glycine max, Zea mays or Arabidopsis thaliana. More preferably, the gene is from Linum usitatissimum or Carthamus tinctorius.

In an embodiment, the second nucleotide sequence comprises a sequence provided as any one of SEQ ID NOs 43 to 85, 100, 101, 104 or 105, or a fragment thereof which is at least 19 nucleotides in length.

As will be apparent, preferred features and characteristics of one aspect of the invention are applicable to many other aspects of the invention. In particular, embodiments of methods of producing oil, seeds and plants comprising said seeds are equally applicable for each aspect.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. Schematic diagram of metabolic routes by which fatty acids that are modified on PC can be transferred to TAGs.

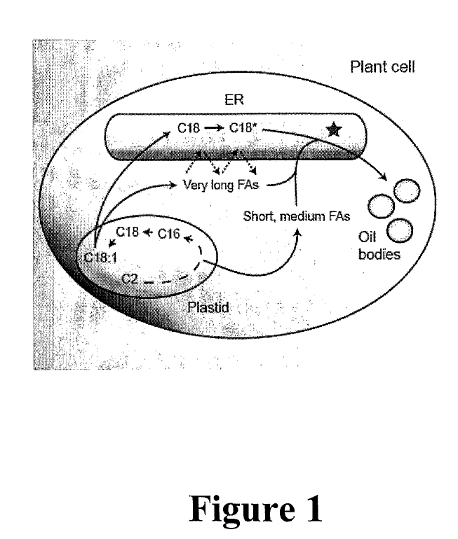

FIG. 2. Schematic representation of the biosynthesis of triacylglycerols.

### KEY TO THE SEQUENCE LISTING

SEQ ID NO:1—Amino acid sequence of Bernardia pulchella diacylglycerol acyltransferase 2 (DGAT2).

SEQ ID NO:2—Amino acid sequence of Bernardia pulchella diacylglycerol acyltransferase 1 (DGAT1).

SEQ ID NO:3—Amino acid sequence of Bernardia pulchella diacylglycerol acyltransferase 3 (DGAT3).

SEQ ID NO:4—Amino acid sequence of Bernardia pulchella phospholipase A2 (PLA2).

SEQ ID NO:5—Amino acid sequence of Euphorbia lagascae phosphatidylcholine diacylglycerol acyltransferase (PDAT).

SEQ ID NO:6—Amino acid sequence of Bernardia pulchella phosphatidylcholine diacylglycerol acyltransferase (PDAT).

SEQ ID NO:7—Amino acid sequence of Bernardia pulchella CDP-choline diacylglycerol choline phosphotransferase (CPT).

SEQ ID NO:8—Amino acid sequence of Bernardia pulchella acyl-CoA:lysophosphatidylcholine acyltransferase 1 (LPCAT1).

SEQ ID NO:9—Amino acid sequence of Bernardia pulchella acyl-CoA:lysophosphatidylcholine acyltransferase 2 (LPCAT2).

SEQ ID NO:10—Amino acid sequence of Bernardia pulchella phospholipase C-a (PLC-a).

SEQ ID NO:11—Amino acid sequence of Bernardia pulchella phospholipase C-b (PLC-b).

SEQ ID NO:12—Partial amino acid sequence of Bernardia pulchella phospholipase C-c (PLC-c).

SEQ ID NO:13—Partial amino acid sequence of Bernardia pulchella phospholipase C-d (PLC-d).

SEQ ID NO:14—Amino acid sequence of Bernardia pulchella phospholipase Dα1 (PLDα1).

SEQ ID NO:15—Amino acid sequence of Bernardia pulchella glycerol-3-phosphate acyltransferase (GPAT).

SEQ ID NO:16—Amino acid sequence of Bernardia pulchella 1-acyl-glycerol-3-phosphate acyltransferase (LPAAT).

SEQ ID NO:17—Amino acid sequence of Bernardia pulchella acyltransferase 1 (AT1).

SEQ ID NO:18—Amino acid sequence of Bernardia pulchella acyltransferase 2 (AT2).

SEQ ID NO:19—Amino acid sequence of Bernardia pulchella acyltransferase 3 (AT3).

SEQ ID NO:20—Amino acid sequence of Bernardia pulchella acyltransferase 4 (AT4).

SEQ ID NO:21—Partial amino acid sequence of Bernardia pulchella epoxygenase-like protein.

SEQ ID NO:22—Amino acid sequence of Bernardia pulchella Δ12 desaturase.

SEQ ID NO:23—Partial amino acid sequence of Bernardia pulchella Δ12 desaturase, or FAD2, -like protein 2.

SEQ ID NO:24—Amino acid sequence of Bernardia pulchella Δ12 desaturase, or FAD2, -like protein 3.

SEQ ID NO:25—Partial amino acid sequence of Bernardia pulchella acyltransferase-like protein 1.

SEQ ID NO:26—Partial amino acid sequence of Bernardia pulchella acyltransferase-like protein 2.

SEQ ID NO:27—Partial amino acid sequence of Bernardia pulchella acyltransferase-like protein 3.

SEQ ID NO:28—Partial amino acid sequence of Bernardia pulchella 3-ketoayl-CoA synthase 4-like protein.

SEQ ID NO:29—Partial amino acid sequence of Bernardia pulchella diacylglycerol acyltransferase-like protein.

SEQ ID NO:30—Amino acid sequence of Bernardia pulchella phospholipase-a (PL-a).

SEQ ID NO:31—Partial amino acid sequence of Bernardia pulchella phospholipase-b (PL-b).

SEQ ID NO:32—Partial amino acid sequence of Bernardia pulchella phospholipase-c (PL-c).

SEQ ID NO:33—Partial amino acid sequence of Bernardia pulchella lipase-d (L-d).

SEQ ID NO:34—Partial amino acid sequence of Bernardia pulchella lipase-e (L-e). SEQ ID NO:35—Partial amino acid sequence of Bernardia pulchella lipase-f (L-f).

SEQ ID NO:36—Partial amino acid sequence of Bernardia pulchella lipase-g (L-g).

SEQ ID NO:37—Partial amino acid sequence of Bernardia pulchella lipase-h (L-h).

SEQ ID NO:38—Amino acid sequence of Bernardia pulchella lipase-i (L-i).

SEQ ID NO:39—Partial amino acid sequence of Bernardia pulchella esterase/lipase/thioesterase-like family protein.

SEQ ID NO:40—Partial amino acid sequence of Bernardia pulchella GDSL-motif lipase/hydrolase-like protein 1.

SEQ ID NO:41—Partial amino acid sequence of Bernardia pulchella GDSL-motif lipase/hydrolase-like protein 2.

SEQ ID NO:42—Partial amino acid sequence of Bernardia pulchella GDSL-motif lipase/hydrolase-like protein 3.

SEQ ID NO:43—cDNA for Bernardia pulchella diacylglycerol acyltransferase 2 (DGAT2). Protein coding sequence is from nucleotide 232 to 1210.

SEQ ID NO:44—cDNA for Bernardia pulchella diacylglycerol acyltransferase 1 (DGAT1). Protein coding sequence is from nucleotide 75 to 1727.

SEQ ID NO:45—cDNA for Bernardia pulchella diacylglycerol acyltransferase 3 (DGAT3). Protein coding sequence is from nucleotide 73 to 1062.

SEQ ID NO:46—cDNA for Bernardia pulchella phospholipase A2 (PLA2). Protein coding sequence is from nucleotide 71 to 535.

SEQ ID NO:47—cDNA for Euphorbia lagascae phosphatidylcholine diacylglycerol acyltransferase (PDAT). Protein coding sequence is from nucleotide 266 to 1801.

SEQ ID NO:48—cDNA for Bernardia pulchella phosphatidylcholine diacylglycerol acyltransferase (PDAT). Protein coding sequence is from nucleotide 208 to 2256.

SEQ ID NO:49—cDNA for Bernardia pulchella CDP-choline diacylglycerol choline phosphotransferase (CPT). Protein coding sequence is from nucleotide 514 to 1683.

SEQ ID NO:50—cDNA for Bernardia pulchella acyl-CoA:lysophosphatidylcholine acyltransferase 1 (LPCAT1). Protein coding sequence is from nucleotide 58 to 1437.

SEQ ID NO:51—cDNA for Bernardia pulchella acyl-CoA:lysophosphatidylcholine acyltransferase 2 (LPCAT2). Protein coding sequence is from nucleotide 139 to 1539.

SEQ ID NO:52—cDNA for Bernardia pulchella phospholipase C-a (PLC-a). Protein coding sequence is from nucleotide 12 to 968.

SEQ ID NO:53—cDNA for Bernardia pulchella phospholipase C-b (PLC-b). Protein coding sequence is from nucleotide 34 to 1299.

SEQ ID NO:54—Partial cDNA for Bernardia pulchella phospholipase C-c (PLC-c). Protein coding sequence is up to and including nucleotide 498.

SEQ ID NO:55—Partial cDNA for Bernardia pulchella phospholipase C-d (PLC-d).

Protein coding sequence is up to and including nucleotide 334. SEQ ID NO:56—cDNA for Bernardia pulchella phospholipase Dα1 (PLDα1). Protein coding sequence is from nucleotide 125 to 2548.

SEQ ID NO:57—cDNA for Bernardia pulchella glycerol-3-phosphate acyltransferase (GPAT). Protein coding sequence is from nucleotide 29 to 1534.

SEQ ID NO:58 cDNA for Bernardia pulchella 1-acyl-glycerol-3-phosphate acyltransferase (LPAAT). Protein coding sequence is from nucleotide 14 to 1393.

SEQ ID NO:59—cDNA for Bernardia pulchella acyltransferase 1 (AT1). Protein coding sequence is from nucleotide 99 to 1607.

SEQ ID NO:60—cDNA for Bernardia pulchella acyltransferase 2 (AT2). Protein coding sequence is from nucleotide 71 to 1393.

SEQ ID NO:61—cDNA for Bernardia pulchella acyltransferase 3 (AT3). Protein coding sequence is from nucleotide 34 to 1419.

SEQ ID NO:62—cDNA for Bernardia pulchella acyltransferase 4 (AT4). Protein coding sequence is from nucleotide 45 to 1569.

SEQ ID NO:63—Partial cDNA for Bernardia pulchella epoxygenase-like protein. Protein coding sequence is up to and including nucleotide 588.

SEQ ID NO:64—cDNA for Bernardia pulchella M2 destaurase. Protein coding sequence is from nucleotide 117 to 1268.

SEQ ID NO:65—Partial cDNA for Bernardia pulchella FAD2-like protein 2. Protein coding sequence is up to and including nucleotide 939.

SEQ ID NO:66—cDNA for Bernardia pulchella FAD2-like protein 3. Protein coding sequence is from nucleotide 111 to 1262.

SEQ ID NO:67—Partial cDNA for Bernardia pulchella acyltransferase-like protein 1. Protein coding sequence is up to and including nucleotide 176.

SEQ ID NO:68—Partial cDNA for Bernardia pulchella acyltransferase-like protein 2. Protein coding sequence is up to and including nucleotide 257.

SEQ ID NO:69—Partial cDNA for Bernardia pulchella acyltransferase-like protein 3. Protein coding sequence is from nucleotide 77.

SEQ ID NO:70—Partial cDNA for Bernardia pulchella 3-ketoayl-CoA synthase 4-like protein. Protein coding sequence is from nucleotide 94.

SEQ ID NO:71—Partial cDNA for Bernardia pulchella diacylglycerol acyltransferase-like protein. Protein coding sequence is up to and including nucleotide 588.

SEQ ID NO:72—cDNA for Bernardia pulchella phospholipase-a (BpPL-a). Protein coding sequence is from nucleotide 17 to 1567.

SEQ ID NO:73—Partial cDNA for Bernardia pulchella phospholipase-a (BpPL-a). Protein coding sequence is from nucleotide 1 to 674. Includes an intron.

SEQ ID NO:74—Partial cDNA for Bernardia pulchella phospholipase-b (BpPL-b). Protein coding sequence is from nucleotide 134.

SEQ ID NO:75—Partial cDNA for Bernardia pulchella phospholipase-c (BpPL-c). Protein coding sequence is from nucleotide 117.

SEQ ID NO:76—Partial cDNA for Bernardia pulchella lipase-d (BpL-d). Protein coding sequence is from nucleotide 200.

SEQ ID NO:77—Partial cDNA for Bernardia pulchella lipase-e (BpL-e). Protein coding sequence is from nucleotide 224.

SEQ ID NO:78—cDNA for Bernardia pulchella lipase-f (BpL-f). Protein coding sequence is from nucleotide 15 to 1133.

SEQ ID NO:79—Partial cDNA for Bernardia pulchella lipase-g (BpL-g). Protein coding sequence is from nucleotide 1 to 842.

SEQ ID NO:80—Partial cDNA for Bernardia pulchella lipase-h (BpL-h). Protein coding sequence is from nucleotide 1 to 482.

SEQ ID NO:81—cDNA for Bernardia pulchella lipase-i (BpL-i). Protein coding sequence is from nucleotide 410.

SEQ ID NO:82—Partial cDNA for Bernardia pulchella esterase/lipase/thioesterase-like family protein. Protein coding sequence is up to and including nucleotide 396.

SEQ ID NO:83—Partial cDNA for Bernardia pulchella GDSL-motif lipase/hydrolase-like protein 1. Protein coding sequence is from nucleotide 244.

SEQ ID NO:84—Partial cDNA for Bernardia pulchella GDSL-motif lipase/hydrolase-like protein 2. Protein coding sequence is from nucleotide 48.

SEQ ID NO:85—Partial cDNA for Bernardia pulchella GDSL-motif lipase/hydrolase-like protein 3. Protein coding sequence is from nucleotide 62.

SEQ ID NO's 86 to 97—Oligonucleotide primers.

SEQ ID NO:98—Amino acid sequence of Bernardia pulchella 1-acyl-glycerol-3-phosphate acyltransferase (LPAAT) 2.

SEQ ID NO:99—Amino acid sequence of Bernardia pulchella 1-acyl-glycerol-3-phosphate acyltransferase (LPAAT) 3.

SEQ ID NO:100—cDNA for Bernardia pulchella 1-acyl-glycerol-3-phosphate acyltransferase (LPAAT) 2. Protein coding sequence is from nucleotide 80 to 1219.

SEQ ID NO:101—cDNA for Bernardia pulchella 1-acyl-glycerol-3-phosphate acyltransferase (LPAAT) 3. Protein coding sequence is from nucleotide 11 to 1064.

SEQ ID NO:102—Amino acid sequence of Bernardia pulchella diacylglycerol acyltransferase-like protein.

SEQ ID NO:103—Amino acid sequence of Bernardia pulchella diacylglycerol acyltransferase-like protein. Variant of SEQ ID NO:102.

SEQ ID NO:104—cDNA for Bernardia pulchella diacylglycerol acyltransferase-like protein. Protein coding sequence is from nucleotide 7 to 984.

SEQ ID NO:105—cDNA for Bernardia pulchella diacylglycerol acyltransferase-like protein. Variant of SEQ ID NO:104. Protein coding sequence is from nucleotide 63 to 1040.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, fatty acid chemistry and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Selected Definitions

As used herein, the term “seedoil” refers to a composition obtained from the seed/grain of a plant which comprises at least 60% (w/w) lipid. Seedoil is typically a liquid at room temperature. Preferably, the lipid predominantly (>50%) comprises fatty acids that are at least 16 carbons in length. More preferably, at least 50% of the total fatty acids in the seedoil are C18 fatty acids. The fatty acids are typically in an esterified form, such as for example as triacylglycerols, acyl-CoA or phospholipid. The fatty acids may be free fatty acids and/or be found esterified such as triacylglycerols (TAGs). In an embodiment, at least 50%, more preferably at least 70%, more preferably at least 80% or at least 90% of the fatty acids in seedoil of the invention can be found as TAGs. Seedoil of the invention can form part of the grain/seed or portion thereof. Alternatively, seedoil of the invention has been extracted from grain/seed. Thus, in an embodiment, “seedoil” of the invention is “substantially purified” or “purified” oil that has been separated from one or more other lipids, nucleic acids, polypeptides, or other contaminating molecules with which it is associated in its native state. It is preferred that the substantially purified oil is at least 60% free, more preferably at least 75% free, and more preferably at least 90% free from other components with which it is naturally associated. Seedoil of the invention may further comprise non-fatty acid molecules such as, but not limited to, sterols. In an embodiment, the seedoil is canola oil (Brassica napus, Brassica rapa ssp.), mustard oil (Brassica juncea), other Brassica oil, sunflower oil (Helianthus annus), linseed oil (Linum usitatissimum), soybean oil (Glycine max), safflower oil (Carthamus tinctorius), corn oil (Zea mays), tobacco oil (Nicotiana tabacum), peanut oil (Arachis hypogaea), palm oil, cottonseed oil (Gossypium hirsutum), coconut oil (Cocos nucifera), avocado oil (Persea americana), olive oil (Olea europaea), cashew oil (Anacardium occidentale), macadamia oil (Macadamia intergrifolia), almond oil (Prunus amygdalus) or Arabidopsis seed oil (Arabidopsis thaliana). Seedoil may be extracted from seed by any method known in the art. This typically involves extraction with nonpolar solvents such as diethyl ether, petroleum ether, chloroform/methanol or butanol mixtures. Lipids associated with the starch in the grain may be extracted with water-saturated butanol. The seedoil may be “de-gummed” by methods known in the art to remove polysaccharides or treated in other ways to remove contaminants or improve purity, stability or colour. The triacylglycerols and other esters in the oil may be hydrolysed to release free fatty acids, or the oil hydrogenated or treated chemically or enzymatically as known in the art.

As used herein, the term “oil” refers to a composition which comprises at least 60% (w/w) lipid. Oil is typically a liquid at room temperature. Preferably, the lipid predominantly comprises fatty acids that are at least 16 carbons in length. The fatty acids are typically in an esterified form, such as for example as triacylglycerols, acyl-CoA or phospholipid. The fatty acids may be free fatty acids and/or be found as triacylglycerols (TAGs). In an embodiment, at least 50%, more preferably at least 70%, more preferably at least 80% of the fatty acids in seedoil of the invention can be found as TAGs. “Oil” of the invention may be “seedoil” if it is obtained from seed. Oil may be present in or obtained from cells, tissues, organs or organisms other than seeds, in which case the oil is not seedoil as defined herein.

As used herein, the term “fatty acid” refers to a carboxylic acid (or organic acid), often with a long aliphatic tail, either saturated or unsaturated. Typically fatty acids have a carbon-carbon bonded chain of at least 8 carbon atoms in length, more preferably at least 12 carbons in length. Most naturally occurring fatty acids have an even number of carbon atoms because their biosynthesis involves acetate which has two carbon atoms. The fatty acids may be in a free state (non-esterified) or in an esterified form such as part of a triglyceride, diacylglyceride, monoacylglyceride, acyl-CoA (thio-ester) bound or other bound form. The fatty acid may be esterified as a phospholipid such as a phosphatidylcholine, phosphatidylethanolamine, phosphatidylserine, phosphatidylglycerol, phosphatidylinositol or diphosphatidylglycerol forms. The terms “fatty acid” and “fatty acids” are generally used interchangeably, however, as the skilled person will appreciate seedoil will comprise more than a single fatty acid molecule and generally more than one type of fatty acid.

Triacylglyceride (TAG) is glyceride in which the glycerol is esterified with three fatty acids. In the Kennedy pathway of TAG synthesis, the precursor sn-glycerol-3-phosphate is esterified by a fatty acid coenzyme A ester in a reaction catalysed by a glycerol-3-phosphate acyltransferase at position sn-1 to form lysophosphatidic acid (LPA), and this is in turn acylated by an acylglycerophosphate acyltransferase in position sn-2 to form phosphatidic acid. The phosphate group is removed by the enzyme phosphatidic phosphohydrolase, and the resultant 1,2-diacyl-sn-glycerol (DAG) is acylated by a diacylglycerol acyltransferase to form the triacyl-sn-glycerol.

“Modified fatty acid” or “modified fatty acids” refers to fatty acids which comprise a functional group which is a hydroxyl group, an epoxy group, an acetylenic group or a conjugated double bond. These types of groups are well known in the art, with an hydroxyl group comprising of an oxygen and hydrogen atom covalently bonded to a carbon group of the carbon chain of the fatty acid; an epoxy group is a three membered ring comprising two carbons atoms and an oxygen atom; an acetylenic group comprises a triple bond between two carbons in the carbon chain of the fatty acid; and conjugated double bond is a system of atoms covalently bonded with alternating single and multiple (for example double) bonds such as —C═C—C═C—C—.

Vernolic acid is cis-12,13-epoxy-octadec-cis-9-enoic acid, whereas ricinoleic acid is 12-hydroxy-9-cis-octadecenoic acid. Preferably, these modified fatty acids form part of a TAG. As used herein, bi-vernoleate and tri-vernoleate refer to TAGs comprising two and three vernolic fatty respectively. Furthermore, bi-ricinoleate and tri-ricinoleate refer to TAGs comprising two and three ricinoleic acids respectively.

As used herein, “the production of triacylglycerols is modified” is a relative term which refers to the total amount of TAGs being produced being modified and/or the chemical composition of the TAGs being produced being modified. In a preferred embodiment, a nucleic acid identified using a method of the invention encodes a polypeptide that increases the production of TAGs comprising a modified fatty acid. In a preferred embodiment, the production is enhanced such that the level of the modified fatty acids comprising the functional group is increased by at least 6% as a percentage of the total fatty acid content after extraction of the total fatty acids with chloroform/methanol.

As used herein, “the production of fatty acid-CoA and/or triacylglycerols is enhanced” is a relative term which refers to the total amount of fatty acid-CoA and/or TAGs being produced being increased. In a preferred embodiment, a nucleic acid identified using a method of the invention encodes a polypeptide that increases the production of fatty acid-CoA and/or TAGs comprising a modified fatty acid.

As used herein, “the fatty acid composition is modified” is a relative term which refers to the total amount of fatty acids being produced being modified and/or the chemical composition of the fatty acids being produced being modified. In a preferred embodiment, a nucleic acid identified using a method of the invention encodes a polypeptide that increases the production of fatty acids comprising a modified fatty acid. More preferably, a nucleic acid identified using a method of the invention encodes a polypeptide that increases the production of TAGs comprising a modified fatty acid. Furthermore, when the nucleic acid encodes an acyltransferase or a phospholipase, it is preferred that the ratio of fatty acid-CoA:fatty acid-PC:triacylglycerol is modified relative, in particular it is preferred that the relative quantity of TAG is increased when compared to fatty acid-PC.

As used herein, the term “transgenic cell with enhanced ability to produce one or more modified fatty acids” is a relative term where the transgenic cell of the invention is compared to the native cell, with the transgenic cell producing more modified fatty acids, or a greater concentration of modified fatty acids present as TAGs (relative to other fatty acids), than the native cell.

As used herein, the term “predominantly C18 fatty acids” means that at least 50%, more preferably at least 60%, more preferably at least 70%, more preferably at least 80%, and even more preferably at least 90%, of the fatty acids in the seedoil or seed are in triglycerides, diacylglycerides and/or monoacylglycerides as C18 fatty acids or derivatives thereof such as modified fatty acids as defined herein, and/or unsaturated fatty acids such as C18:1 and/or C18:2.

“Saturated fatty acids” do not contain any double bonds or other functional groups along the chain. The term “saturated” refers to hydrogen, in that all carbons (apart from the carboxylic acid [—COOH] group) contain as many hydrogens as possible. In other words, the omega (ω) end contains 3 hydrogens (CH3-) and each carbon within the chain contains 2 hydrogens (—CH2-).

“Unsaturated fatty acids” are of similar form to saturated fatty acids, except that one or more alkene functional groups exist along the chain, with each alkene substituting a singly-bonded “—CH2-CH2-” part of the chain with a doubly-bonded “—CH═CH—” portion (that is, a carbon double bonded to another carbon). The two next carbon atoms in the chain that are bound to either side of the double bond can occur in a cis or trans configuration.

As used herein, the terms “monounsaturated fatty acid” refers to a fatty acid which comprises at least 12 carbon atoms in its carbon chain and only one alkene group in the chain. As used herein, the terms “polyunsaturated fatty acid” or “PUFA” refer to a fatty acid which comprises at least 12 carbon atoms in its carbon chain and at least two alkene groups (carbon-carbon double bonds). Ordinarily, the number of carbon atoms in the carbon chain of the fatty acids refers to an unbranched carbon chain. If the carbon chain is branched, the number of carbon atoms excludes those in sidegroups. In one embodiment, the long-chain polyunsaturated fatty acid is an ω3 fatty acid, that is, having a desaturation (carbon-carbon double bond) in the third carbon-carbon bond from the methyl end of the fatty acid. In another embodiment, the long-chain polyunsaturated fatty acid is an ω6 fatty acid, that is, having a desaturation (carbon-carbon double bond) in the sixth carbon-carbon bond from the methyl end of the fatty acid.

As used herein, the terms “long-chain polyunsaturated fatty acid” or “LC-PUFA” refer to a fatty acid which comprises at least 20 carbon atoms in its carbon chain and at least two carbon-carbon double bonds.

The term “epoxygenase” or “fatty acid epoxygenase” as used herein refers to an enzyme that introduces an epoxy group into a fatty acid resulting in the production of an epoxy fatty acid. In preferred embodiment, the epoxy group is introduced at the 12th carbon on a fatty acid chain, in which case the epoxygenase is a Δ12-epoxygenase, especially of a C16 or C18 fatty acid chain. The epoxygenase may be a Δ9-epoxygenase, a Δ15 epoxygenase, or act at a different position in the acyl chain as known in the art. The epoxygenase may be of the P450 class. Preferred epoxygenases are of the mono-oxygenase class as described in WO98/46762. Numerous epoxygenases or presumed epoxygenases have been cloned and are known in the art. Further examples of expoxygenases include proteins comprising an amino acid sequence provided in SEQ ID NO:21, polypeptides encoded by genes from Crepis paleastina (Accession No. CAA76156, Lee et al., 1998), Stokesia laevis (AAR23815, Hatanaka et al., 2004) (monooxygenase type), Euphorbia lagascae (AAL62063) (P450 type), human CYP2J2 (arachidonic acid epoxygenase, U37143); human CYPIA1 (arachidonic acid epoxygenase, K03191), as well as variants and/or mutants thereof.

“Hydroxylase” or “fatty acid hydroxylase” as used herein, refers to an enzyme that introduces a hydroxyl group into a fatty acid resulting in the production of a hydroxylated fatty acid. In a preferred embodiment, the hydroxyl group is introduced at the 2nd, 12th and/or 17th carbon on a C18 fatty acid chain. Preferably, the hydroxyl group is introduced at the 12^{th} carbon, in which case the hydroxylase is a Δ12-hydroxylase. In another preferred embodiment, the hydroxyl group is introduced at the 15th carbon on a C16 fatty acid chain. Hydroxylases may also have enzyme activity as a fatty acid desaturase. Examples of genes encoding Δ12-hydroxylases include those from Ricinus communis (AAC9010, van de Loo 1995); Physaria lindheimeri, (ABQ01458, Dauk et al., 2007); Lesquerella fendleri, (AAC32755, Broun et al., 1998); Daucus carota, (AAK30206); fatty acid hydroxylases which hydroxylate the terminus of fatty acids, for example: A. thaliana CYP86A1 (P48422, fatty acid ω-hydroxylase); Vicia sativa CYP94A1 (P98188, fatty acid ω-hydroxylase); mouse CYP2E1 (X62595, lauric acid ω-1 hydroxylase); rat CYP4A1 (M57718, fatty acid ω-hydroxylase), as well as as variants and/or mutants thereof.

As used herein, the term “conjugase” or “fatty acid conjugase” refers to an enzyme capable of forming a conjugated bond in the acyl chain of a fatty acid. Examples of conjugases include those encoded by genes from Calendula officinalis (AF343064, Qiu et al., 2001); Vernicia fordii (AAN87574, Dyer et al., 2002); Punica granatum (AY178446, Iwabuchi et al., 2003) and Trichosanthes kirilowii (AY178444, Iwabuchi et al., 2003); as well as as variants and/or mutants thereof.

As used herein, the term “acetylenase” or “fatty acid acetylenase” refers to an enzyme that introduces a triple bond into a fatty acid resulting in the production of an acetylenic fatty acid. In a preferred embodiment, the triple bond is introduced at the 2nd, 6th, 12th and/or 17th carbon on a C18 fatty acid chain. Examples acetylenases include those from Helianthus annuus (AA038032, ABC59684), as well as as variants and/or mutants thereof.

As used herein, the term “diacylglycerol acyltransferase” (EC 2.3.1.20; DGAT) refers to a protein which transfers a fatty acyl group from acyl-CoA or diacylglycerol to a diacylglycerol substrate to produce a triacylglycerol. Thus, the term “diacylglycerol acyltransferase activity” refers to the transfer of an acyl group to diacylglycerol to produce triacylglycerol. There are three known types of DGAT referred to as DGAT1, DGAT2 and soluble DGAT (DGAT3) respectively. DGAT1 polypeptides typically have 10 transmembrane domains, DGAT2 typically have 2 transmembrane domains, whilst DGAT3 is typically soluble. Examples of DGAT1 polypeptides include proteins comprising an amino acid sequence provided in SEQ ID NO:2, polypeptides encoded by DGAT1 genes from Aspergillus fumigatus (Accession No. XP_{—}755172), Arabidopsis thaliana (CAB44774), Ricinus communis (AAR11479), Vernicia fordii (ABC94472), Vernonia galamensis (ABV21945, ABV21946), Euonymus alatus (AAV31083), Caenorhabditis elegans (AAF82410), Rattus norvegicus (NP_{—}445889), Homo sapiens (NP_{—}036211), as well as variants and/or mutants thereof. Examples of DGAT2 polypeptides include proteins comprising an amino acid sequence provided in SEQ ID NO:1, polypeptides encoded by DGAT2 genes from Arabidopsis thaliana (Accession No. NP_{—}566952), Ricinus communis (AAY16324), Vernicia fordii (ABC94474), Mortierella ramanniana (AAK84179), Homo sapiens (Q96PD7, Q58HT5), Bos taurus (Q70VD8), Mus musculus (AAK84175), as well as variants and/or mutants thereof. Examples of DGAT3 polypeptides include proteins comprising an amino acid sequence provided in SEQ ID NO:3, polypeptides encoded by DGAT3 genes from peanut (Arachis hypogaea, Saha, et al., 2006), as well as variants and/or mutants thereof.

As used herein, the term “phospholipase A₂” (PLA₂) refers to a protein which hydrolyzes the sn2-acyl bond of phospholipids to produce free fatty acid and lysophospholipids. Thus, the term “phospholipase A₂ activity” refers to the hydrolysis of the sn2-acyl bond of phospholipids to produce free fatty acid and lysophospholipids. Examples of phospholipase A₂ polypeptides include proteins comprising an amino acid sequence provided in SEQ ID NO:4, polypeptides encoded by PLA₂ genes from Arabidopsis such as −α (At2g06925, AY136317), AtsPLA₂-β (At2g19690, AY136317), AtsPLA₂-γ (At4g29460, AY148346), AtsPLA₂-δ (At4g29470, AY148347) and PLA₂s (At3g45880, AK226677 and At1g61850, NM_{—}104867), as well as variants and/or mutants thereof.

As used herein, the term “phosphatidylcholine diacylglycerol acyltransferase” (PDAT) refers to a protein which transfers an acyl group from phosphatidylcholine to diacylglycerol. Thus, the term “phosphatidylcholine diacylglycerol acyltransferase activity” refers to the transfer of an acyl group from phosphatidylcholine onto diacylglycerol to produce triacylglycerol. Examples of phosphatidylcholine diacylglycerol acyltransferase polypeptides include proteins comprising an amino acid sequence provided in SEQ ID NO's 5 and 6, as well as variants and/or mutants thereof.

As used herein, the term “CDP-choline diacylglycerol choline phosphotransferase” (CPT), refers to a protein which reversibly converts phosphatidylcholine into diacylglycerol. Thus, the term “CDP-choline diacylglycerol choline phosphotransferase activity” refers to the reversible conversion of phosphatidylcholine into diacylglycerol. Examples of CDP-choline diacylglycerol choline phosphotransferase polypeptides include proteins comprising an amino acid sequence provided in SEQ ID NO:7, as well as variants and/or mutants thereof.

As used herein, the term “acyl-CoA:lysophosphatidylcholine acyltransferase” (EC 2.3.1.23; LPCAT) refers to a protein which reversibly catalyzes the acyl-CoA-dependent acylation of lysophophatidylcholine to produce phosphatidylcholine and CoA. Thus, the term “acyl-CoA:lysophosphatidylcholine acyltransferase activity” refers to the reversible acylation of lysophophatidylcholine to produce phosphatidylcholine and CoA. Examples of acyl-CoA:lysophosphatidylcholine acyltransferase polypeptides include proteins comprising an amino acid sequence provided in SEQ ID NOs 8 and 9, as well as variants and/or mutants thereof.

As used herein, the term “phospholipase C” (PLC) refers to a protein which hydrolyzes PIP₂ to produce diacylglycerol. Thus, the term “phospholipase C activity” refers to the hydrolysis of PIP₂ to produce diacylglycerol. Examples of phospholipase C polypeptides include proteins comprising an amino acid sequence provided in SEQ ID Nos 10 to 13, as well as variants and/or mutants thereof.

As used herein, the term “phospholipase D” (PLD) refers to a protein which hydrolyzes phosphatidylcholine to produce phosphatidic acid and a choline headgroup. Thus, the term “phospholipase D activity” refers to the hydrolysis of phosphatidylcholine to produce phosphatidic acid and a choline headgroup. Examples of phospholipase D polypeptides include proteins comprising an amino acid sequence provided in SEQ ID NO:14, as well as variants and/or mutants thereof.

As used herein, the term “glycerol-3-phosphate acyltransferase” (GPAT) refers to a protein which acylates sn-glycerol-3-phosphate to form 1-acyl-sn-glycerol-3-phosphate. Thus, the term “glycerol-3-phosphate acyltransferase activity” refers to the acylation of sn-glycerol-3-phosphate to form 1-acyl-sn-glycerol-3-phosphate. Examples of glycerol-3-phosphate acyltransferase polypeptides include proteins comprising an amino acid sequence provided in SEQ ID NO:15, as well as variants and/or mutants thereof.

As used herein, the term “1-acyl-glycerol-3-phosphate acyltransferase” (LPAAT) refers to a protein which acylates sn-1-acyl-glycerol-3-phosphate at the sn-2 position to form phosphatidic acid. Thus, the term “1-acyl-glycerol-3-phosphate acyltransferase activity” refers to the acylation of sn-1-acyl-glycerol-3-phosphate at the sn-2 position to produce phosphatidic acid. Examples of 1-acyl-glycerol-3-phosphate acyltransferase polypeptides include proteins comprising the amino acid sequences provided in SEQ ID NO:16, 98 and 99, as well as variants and/or mutants thereof.

As used herein, the term “acyltransferase” refers to a protein which transfers acyl groups from molecule to another. Thus, the term “acyltransferase activity” refers to the transfer of acyl groups from one molecule to another. Examples of acyltransferase polypeptides include proteins comprising an amino acid sequence provided in SEQ ID NOs 17 to 20, 25 to 27 and 29, as well as variants and/or mutants thereof.

As used herein, the term “3-ketoacyl-CoA synthase” refers to a protein which catalyzes the condensation of malonyl-CoA with acyl-CoA to produce 3-ketoacyl-CoA. Thus, the term “3-ketoacyl-CoA synthase activity” refers to the condensation of malonyl-CoA with acyl-CoA to produce 3-ketoacyl-CoA. Examples of 3-ketoacyl-CoA synthase polypeptides include proteins comprising an amino acid sequence provided in SEQ ID NO:28, as well as variants and/or mutants thereof.

As used herein, the term “phospholipase” refers to a protein which hydrolyzes specific ester bonds in phospholipids. Thus, the term “phospholipase activity” refers to the hydrolysis of specific ester bonds in phospholipids. Examples of acyltransferase polypeptides include proteins comprising an amino acid sequence provided in SEQ ID NOs 30 to 32, as well as variants and/or mutants thereof.

As used herein, the term “lipase” refers to a protein which hydrolyzes fats into glycerol and fatty acids. Thus, the term “lipase activity” refers to the hydrolysis of fats into glycerol and fatty acids. Examples of acyltransferase polypeptides include proteins comprising an amino acid sequence provided in SEQ ID NOs 33 to 42, as well as variants and/or mutants thereof.

As used herein, a “desaturase”, “fatty acid desaturase” or variations thereof is an enzyme which removes two hydrogen atoms from the carbon chain of the fatty acid creating a carbon-carbon double bond. Desaturases are classified as; i) delta—indicating that the double bond is created at a fixed position from the carboxyl group of a fatty acid (for example, Δ12 desaturase creates a double bond at the 12th position from the carboxyl end), or ii) omega (e.g. ω3 desaturase)—indicating the double bond is created at a specific position from the methyl end of the fatty acid. Examples of desaturases include those described in WO 2005/103253.

Biochemical evidence suggests that the fatty acid elongation consists of 4 steps: condensation, reduction, dehydration and a second reduction. In the context of this invention, an “elongase” refers to the polypeptide that catalyses the condensing step in the presence of the other members of the elongation complex, under suitable physiological conditions. It has been shown that heterologous or homologous expression in a cell of only the condensing component (“elongase”) of the elongation protein complex is required for the elongation of the respective acyl chain. Thus the introduced elongase is able to successfully recruit the reduction and dehydration activities from the transgenic host to carry out successful acyl elongations. The specificity of the elongation reaction with respect to chain length and the degree of desaturation of fatty acid substrates is thought to reside in the condensing component. This component is also thought to be rate limiting in the elongation reaction. Two groups of condensing enzymes have been identified so far. The first are involved in the extension of saturated and monounsaturated fatty acids (C18-22) such as, for example, the FAE1 gene of Arabidopsis. An example of a product formed is erucic acid (22:1) in Brassicas. This group are designated the FAE-like enzymes and do not appear to have a role in LC-PUFA biosynthesis. The other identified class of fatty acid elongases, designated the ELO family of elongases, are named after the ELO genes whose activities are required for the synthesis of the very long-chain fatty acids of sphingolipids in yeast. Apparent paralogs of the ELO-type elongases isolated from LC-PUFA synthesizing organisms like algae, mosses, fungi and nematodes have been shown to be involved in the elongation and synthesis of LC-PUFA. Examples of elongases include those described in WO 2005/103253.

As used herein, the term “an exogenous polynucleotide which down regulates the production and/or activity of an endogenous enzyme” or variations thereof, refers to a polynucleotide that encodes an RNA molecules that down regulates the production and/or activity (for example, encoding an siRNA), or the exogenous polynucleotide itself down regulates the production and/or activity (for example, an siRNA is delivered to directly to, for instance, a cell).

The term “plant” includes whole plants, vegetative structures (for example, leaves, stems), roots, floral organs/structures, seed (including embryo, endosperm, and seed coat), plant tissue (for example, vascular tissue, ground tissue, and the like), cells and progeny of the same. The plant, seed, plant part or plant cells may be, or from, monocotyledonous plants or preferably dicotyledonous plants.

A “transgenic cell”, “genetically modified cell” or variations thereof refers to a cell that contains a gene construct (“transgene”) not found in a wild-type cell of the same species, variety or cultivar.

A “transgenic seed”, “genetically modified seed” or variations thereof refers to a seed that contains a gene construct (“transgene”) not found in a wild-type seed from the same species, variety or cultivar of plant.

A “transgenic plant”, “genetically modified plant” or variations thereof refers to a plant that contains a gene construct (“transgene”) not found in a wild-type plant of the same species, variety or cultivar.

A “transgene” as referred to herein has the normal meaning in the art of biotechnology and includes a genetic sequence which has been produced or altered by recombinant DNA or RNA technology and which has been introduced into the plant or other cell. The transgene may include genetic sequences derived from a plant cell. Typically, the transgene has been introduced into the plant or other cell by human manipulation such as, for example, by transformation but any method can be used as one of skill in the art recognizes.

“Grain” as used herein generally refers to mature, harvested grain but can also refer to grain after imbibition or germination, according to the context. Mature grain commonly has a moisture content of less than about 18-20%. “Seed” as used herein includes mature seed such as is typically harvested from a plant and developing seed as is typically found in a plant during growth. Mature seed is typically dormant i.e. in a resting state.

As used herein, the term “wild-type” or variations thereof refers to a cell, tissue, seed or plant that has not been modified according to the invention. “Isogenic” refers to a cell, tissue, seed or plant which differs from a reference cell, tissue, seed or plant at one or more, generally not more than a few such as two, three or four, genetic loci, resulting in an alteration of one or more traits. The genetic loci(us) may have a single gene or genetic construct, or multiple genes or genetic constructs (generally not more than a few such as two, three or four), typically a transgene(s). A “corresponding isogenic” cell, tissue, seed or plant as used herein refers to a second cell, tissue, seed or plant which lacks the gene(s) or constructs, which differs from the first cell, tissue, seed or plant essentially by only that gene(s) or construct(s), and which typically has been treated in the same manner e.g. temperature, culture conditions etc, as the first. Isogenic wildtype cells, tissue or plants may be used as controls to compare levels of expression of an exogenous nucleic acid or the extent and nature of trait modification with cells, tissue or plants modified as described herein.

“Operably linked” as used herein refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of at least about 2 kb on either end and which are involved in expression of the gene. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, the term “can be isolated from” means that the polynucleotide or encoded polypeptide is naturally produced by an organism, particularly Bernardia sp., such as Bernardia pulchella.

The term “extract” refers to any part of the cell or organism such as a plant. An “extract” typically involves the disruption of cells and possibly the partial purification of the resulting material. Naturally, the “extract” will comprise at least one modified fatty acid. Extracts can be prepared using standard techniques of the art.

As used herein, the phrase “does not significantly effect the production and/or activity of an enzyme encoded by a transgene” means that the level of activity of the enzyme is at least 75%, more preferably at least 90%, of the level of an isogenic transgenic cell lacking the exogenous polynucleotide that down regulates the production and/or activity of an endogenous enzyme.

As used herein, the term “a region which is not conserved between the first and second nucleotide sequences” refers to portion of the first sequence which is less than 50% identical, more preferably less than 30% identical, over a contiguous stretch of at least 19 nucleotides to any region of the second sequence.

As used herein, the term “similar function” refers to orthologous genes from different plant species which have evolved from a common ancestor. In a preferred embodiment, the enzymes encoded by the orthologs have the same activity accept that the enzyme encoded by the second sequence nucleotide sequence (or encoded by mRNA which comprises the second sequence nucleotide sequence) has a greater level of activity on and/or using modified fatty acids than the enzyme encoded by the first sequence nucleotide sequence (or encoded by mRNA which comprises the first sequence nucleotide sequence). Such enzymes encoded by the orthologous genes will typically have the same Enzyme Commission number (EC number).

### Cells

Suitable cells of the invention include any cell that can be transformed with a polynucleotide encoding a polypeptide/enzyme described herein, and which is thereby capable of being used for producing modified fatty acids. Host cells into which the polynucleotide(s) are introduced can be either untransformed cells or cells that are already transformed with at least one nucleic acid molecule. Such nucleic acid molecule may be related to modified fatty acids synthesis, TAG synthesis, or unrelated. Host cells of the present invention either can be endogenously (i.e., naturally) capable of producing proteins of the present invention or can be capable of producing such proteins only after being transformed with at least one nucleic acid molecule.

The cells may be prokaryotic or eukaryotic. Host cells of the present invention can be any cell capable of producing at least one protein described herein, and include bacterial, fungal (including yeast), parasite, arthropod, animal and plant cells. Preferred cells are eukaryotic cells, more preferred cells are yeast and plant cells. In a preferred embodiment, the plant cells are seed cells. The cells may be in cell culture. The cells may be isolated cells, or alternatively, cells that are or were part of a multicellular organism such as a plant or fungus. The cells may be comprised in a plant part such as a seed. The organism may be non-human.

In one particularly preferred embodiment, the cells may be of an organism suitable for fermentation. As used herein, the term the “fermentation process” refers to any fermentation process or any process comprising a fermentation step. A fermentation process includes, without limitation, fermentation processes used to produce alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, beta-carotene); and hormones. Fermentation processes also include fermentation processes used in the consumable alcohol industry (e.g., beer and wine), dairy industry (e.g., fermented dairy products), leather industry and tobacco industry. Preferred fermentation processes include alcohol fermentation processes, as are well known in the art. Preferred fermentation processes are anaerobic fermentation processes, as are well known in the art.

Suitable fermenting cells, typically microorganisms are able to ferment, i.e., convert, sugars, such as glucose or maltose, directly or indirectly into the desired fermentation product. Examples of fermenting microorganisms include fungal organisms, such as yeast. As used herein, “yeast” includes Saccharomyces spp., Saccharomyces cerevisiae, Saccharomyces carlbergensis, Candida spp., Kluveromyces spp., Pichia spp., Hansenula spp., Trichoderma spp., Lipomyces starkey, and Yarrowia lipolytica. Preferred yeast includes strains of the Saccharomyces spp., and in particular, Saccharomyces cerevisiae. Commercially available yeast include, e.g., Red Star/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties).

In one embodiment, the cell is an animal cell or an algal cell. The animal cell may be of any type of animal such as, for example, a non-human animal cell, a non-human vertebrate cell, a non-human mammalian cell, or cells of aquatic animals such as fish or crustacea, invertebrates, insects, etc.

An example of a bacterial cell useful as a host cell of the present invention is Synechococcus spp. (also known as Synechocystis spp.), for example Synechococcus elongatus.

### Levels of Modified Fatty Acids Produced

The levels of the modified fatty acids produced in the transgenic cells are of importance. The levels may be expressed as a composition (in percent) of the total fatty acid content of the oil that is a particular MFA or group MFAs or other which may be determined by methods known in the art. For example, total lipid may be extracted from the cells, tissues or organisms and the fatty acid converted to methyl esters before analysis by gas chromatography (GC). Such techniques are described in Example 1. The peak position in the chromatogram may be used to identify each particular fatty acid, and the area under each peak integrated to determine the amount. As used herein, unless stated to the contrary, the percentage of particular fatty acid in a sample is determined as the area under the peak for that fatty acid as a percentage of the total area for fatty acids in the chromatogram. This corresponds essentially to a percentage (mol %). The identity of fatty acids may be confirmed by GC-MS, as described in Example 1.

In certain embodiments, at least 23% (mol %), more preferably at least 27%, at least 28%, at least 29%, at least 30% or at least 31% of the fatty acid content of the oil produced by the seed, cell, plant or organism of the invention, or in the seedoil, comprises the functional group.

In other embodiments of the seed, seedoil, cell, plant or organism of the invention, or the methods of the invention, at least 4% (mol %), more preferably at least 10% (mol %), of fatty acids esterified at the sn-3 position of total triacylglycerols comprise the functional group.

In other embodiments of the seed, seedoil, cell, plant or organism of the invention, at least 4% (mol %), more preferably at least 10% (mol %), at least 20%, at least 30%, at least 40%, or at least 50% of fatty acids esterified at the sn-2 position of total triacylglycerols comprise the functional group.

In other embodiments of the seed, seedoil, cell, plant or organism of the invention, at least 4% (mol %), more preferably at least 10% (mol %), of fatty acids esterified at the sn-1 position of total triacylglycerols comprise the functional group.

In other embodiments of the seed, seedoil, cell, plant or organism of the invention, at least 10%, more preferably at least 20%, of the oil produced by the seed, cell, plant or organism, or in the seedoil, is bi-vernoleate or bi-ricinoleate, or a combination thereof.

In other embodiments of the seed, seedoil, cell, plant or organism of the invention, at least 4%, more preferably at least 10%, of the oil produced by the seed, cell, plant or organism, or in the seedoil, is tri-vernoleate or tri-ricinoleate, or a combination thereof.

In other embodiments, the molar ratio in the oil produced by the seed, cell, plant or organism, or in the seedoil, of the fatty acids with the functional group to fatty acids lacking the functional group is at least 23:77, more preferably at least 27:73 and even more preferably at least 31:69.

In a further aspect, a transgenic Carthamus tinctorius (www.ncbi.nlm.nih.gov/Taxonomy/Browser/wwwtax.cgi?mode=Info&id=4222&1v1=3&lin=f&keep=1&srchmode=1&unlock) seed of the invention has at least 17% (mol %), more preferably at least 23%, of the total fatty acid content of the seedoil as vernolic acid and/or ricinoleic acid.

In a further aspect, a transgenic Gossypium hirsutum (www.ncbi.nlm.nih.gov/Taxonomy/Browser/wwwtax.cgi?id=3635) seed of the invention has at least 17% (mol %), more preferably at least 23%, of the total fatty acid content of the seedoil as vernolic acid and/or ricinoleic acid.

In a further aspect, a transgenic Brassica sp seed of the invention has at least 15% (mol %), more preferably at least 23%, of the total fatty acid content of the seedoil as vernolic acid and/or ricinoleic acid.

In a further aspect, a transgenic Linum usitatissimum (www.ncbi.nlm.nih.gov/Taxonomy/Browser/wwwtax.cgi?id=4006) seed of the invention has at least 15% (mol %), more preferably at least 23%, of the total fatty acid content of the seedoil as vernolic acid and/or ricinoleic acid.

An aspect of the invention relates to a method of enhancing the production of one or more modified fatty acids. In this aspect, it is preferred that production is enhanced such that the level of the modified fatty acids comprising the functional group in the oil of the tissue or organ is increased by at least 6%, more preferably at least 8%, as a percentage of the total fatty acid content of the plant tissue or organ after extraction of the total fatty acids from the tissue or organ with chloroform/methanol, and wherein the at least 6% increase, more preferably at least 8%, is relative to the level of the total fatty acids in a corresponding tissue or organ having the first exogenous polynucleotide but lacking the second exogenous polynucleotide.

A further aspect of the invention relates to the efficiency of conversion of the fatty acid to the modified fatty acid in the cell, tissue, seed, plant or other organism. The efficiency of conversion as used herein may be calculated as the percentage of the MFA/percentage of MFA+percentage of the substrate FA (unmodified FA). It is preferred that the efficiency of conversion is at least 25%, more preferably at least 30% and even more preferably at least 35%.

### Polypeptides

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably.

The % identity of a polypeptide is determined by GAP (Needleman and Wunsch, 1970) analysis (GCG program) with a gap creation penalty=5, and a gap extension penalty=0.3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over their entire length.

As used herein a “biologically active” fragment is a portion of a polypeptide of the invention which maintains a defined activity of the full-length polypeptide. Biologically active fragments can be any size as long as they maintain the defined activity. Preferably, the biologically active fragment maintains at least 10% of the activity of the full length protein.

With regard to a defined polypeptide/enzyme, it will be appreciated that % identity figures higher than those provided above will encompass preferred embodiments. Thus, where applicable, in light of the minimum % identity figures, it is preferred that the polypeptide/enzyme comprises an amino acid sequence which is at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a preferred embodiment, the present invention provides a substantially purified and/or recombinant polypeptide comprising amino acids having a sequence as provided in SEQ ID NO:1, a biologically active fragment thereof, or an amino acid sequence which is at least 69% identical to SEQ ID NO:1, wherein the polypeptide has diacylglycerol acyltransferase activity. In a preferred embodiment, the polypeptide has 2 membrane spanning domains.

In another embodiment, the present invention provides a substantially purified and/or recombinant polypeptide comprising amino acids having a sequence as provided in SEQ ID NO:2, a biologically active fragment thereof, or an amino acid sequence which is at least 65% identical to SEQ ID NO:2, wherein the polypeptide has diacylglycerol acyltransferase activity. In a preferred embodiment, the polypeptide has 10 membrane spanning domains.

In another embodiment, the present invention provides a substantially purified and/or recombinant polypeptide comprising amino acids having a sequence as provided in SEQ ID NO:3, a biologically active fragment thereof, or an amino acid sequence which is at least 34% identical to SEQ ID NO:3, wherein the polypeptide has diacylglycerol acyltransferase activity. Preferably, the polypeptide is soluble.

In another embodiment, the present invention provides a substantially purified and/or recombinant polypeptide comprising amino acids having a sequence as provided in SEQ ID NO:4, a biologically active fragment thereof, or an amino acid sequence which is at least 30% identical to SEQ ID NO:4, wherein the polypeptide has phospholipase A2 activity.

In another embodiment, the present invention provides a substantially purified and/or recombinant polypeptide comprising amino acids having a sequence as provided in SEQ ID NO:5, a biologically active fragment thereof, or an amino acid sequence which is at least 51% identical to SEQ ID NO:5, wherein the polypeptide has phoshotidylcholine diacylglycerol acyltransferase activity.

In another embodiment, the present invention provides a substantially purified and/or recombinant polypeptide comprising amino acids having a sequence as provided in SEQ ID NO:6, a biologically active fragment thereof, or an amino acid sequence which is at least 77% identical to SEQ ID NO:6, wherein the polypeptide has phoshotidylcholine diacylglycerol acyltransferase activity.

In another embodiment, the present invention provides a substantially purified and/or recombinant polypeptide comprising amino acids having a sequence as provided in SEQ ID NO:7, a biologically active fragment thereof, or an amino acid sequence which is at least 79% identical to SEQ ID NO:7, wherein the polypeptide has CDP-choline diacylglycerol choline phosphotransferase activity.

In another embodiment, the present invention provides a substantially purified and/or recombinant polypeptide comprising amino acids having a sequence as provided in any one of SEQ ID NOs: 8 or 9, a biologically active fragment thereof, or an amino acid sequence which is at least 75% identical to any one or more of SEQ ID NOs: 8 or 9, wherein the polypeptide has acyl-CoA:lysophosphatidylcholine acyltransferase activity.

In another embodiment, the present invention provides a substantially purified and/or recombinant polypeptide comprising amino acids having a sequence as provided in SEQ ID NO:10, a biologically active fragment thereof, or an amino acid sequence which is at least 80% identical to SEQ ID NO:10, wherein the polypeptide has phospholipase C activity.

In another embodiment, the present invention provides a substantially purified and/or recombinant polypeptide comprising amino acids having a sequence as provided in SEQ ID NO:11, a biologically active fragment thereof, or an amino acid sequence which is at least 66% identical to SEQ ID NO:11, wherein the polypeptide has phospholipase C activity.

In another embodiment, the present invention provides a substantially purified and/or recombinant polypeptide comprising amino acids having a sequence as provided in SEQ ID NO:12, a biologically active fragment thereof, or an amino acid sequence which is at least 58% identical to SEQ ID NO:12, wherein the polypeptide has phospholipase C activity.

In another embodiment, the present invention provides a substantially purified and/or recombinant polypeptide comprising amino acids having a sequence as provided in SEQ ID NO:13, a biologically active fragment thereof, or an amino acid sequence which is at least 79% identical to SEQ ID NO:13, wherein the polypeptide has phospholipase C activity.

In another embodiment, the present invention provides a substantially purified and/or recombinant polypeptide comprising amino acids having a sequence as provided in SEQ ID NO:14, a biologically active fragment thereof, or an amino acid sequence which is at least 92% identical to SEQ ID NO:14, wherein the polypeptide has phospholipase D activity.

In another embodiment, the present invention provides a substantially purified and/or recombinant polypeptide comprising amino acids having a sequence as provided in SEQ ID NO:15, a biologically active fragment thereof, or an amino acid sequence which is at least 81% identical to SEQ ID NO:15, wherein the polypeptide has glycerol-3-phosphate acyltransferase activity.

In another embodiment, the present invention provides a substantially purified and/or recombinant polypeptide comprising amino acids having a sequence as provided in SEQ ID NO:16, 98 or 99, a biologically active fragment thereof, or an amino acid sequence which is at least 36% identical to one or more of SEQ ID NO:16, 98 or 99, wherein the polypeptide has 1-acyl-glycerol-3-phosphate acyltransferase activity.

In another embodiment, the present invention provides a substantially purified and/or recombinant polypeptide comprising amino acids having a sequence as provided in SEQ ID NO:17, a biologically active fragment thereof, or an amino acid sequence which is at least 85% identical to SEQ ID NO:17, wherein the polypeptide has acyltransferase activity.

In another embodiment, the present invention provides a substantially purified and/or recombinant polypeptide comprising amino acids having a sequence as provided in SEQ ID NO:18, a biologically active fragment thereof, or an amino acid sequence which is at least 75% identical to SEQ ID NO:18, wherein the polypeptide has acyltransferase activity.

In another embodiment, the present invention provides a substantially purified and/or recombinant polypeptide comprising amino acids having a sequence as provided in SEQ ID NO:19, a biologically active fragment thereof, or an amino acid sequence which is at least 89% identical to SEQ ID NO:19, wherein the polypeptide has acyltransferase activity.

In another embodiment, the present invention provides a substantially purified and/or recombinant polypeptide comprising amino acids having a sequence as provided in SEQ ID NO:20, a biologically active fragment thereof, or an amino acid sequence which is at least 82% identical to SEQ ID NO:20, wherein the polypeptide has acyltransferase activity.

In another embodiment, the present invention provides a substantially purified and/or recombinant polypeptide comprising amino acids having a sequence as provided in SEQ ID NO:21, a biologically active fragment thereof, or an amino acid sequence which is at least 34% identical to SEQ ID NO:21, wherein the polypeptide has fatty acid epoxygenase activity.

In another embodiment, the present invention provides a substantially purified and/or recombinant polypeptide comprising amino acids having a sequence as provided in SEQ ID NO:22, a biologically active fragment thereof, or an amino acid sequence which is at least 79% identical to SEQ ID NO:22, wherein the polypeptide has Δ12 desaturase activity.

In another embodiment, the present invention provides a substantially purified and/or recombinant polypeptide comprising amino acids having a sequence as provided in SEQ ID NO:23, a biologically active fragment thereof, or an amino acid sequence which is at least 74% identical to SEQ ID NO:23, wherein the polypeptide has fatty acid modifying activity.

In an embodiment, the fatty acid modifying activity is Δ12 desaturase activity.

In another embodiment, the present invention provides a substantially purified and/or recombinant polypeptide comprising amino acids having a sequence as provided in SEQ ID NO:24, a biologically active fragment thereof, or an amino acid sequence which is at least 79% identical to SEQ ID NO:24, wherein the polypeptide has fatty acid modifying activity.

In another embodiment, the present invention provides a substantially purified and/or recombinant polypeptide comprising amino acids having a sequence as provided in any one or more of SEQ ID NOs 25, 26 and 27, a biologically active fragment thereof, or an amino acid sequence which is at least 30% identical to any one or more of SEQ ID NOs 25, 26 and 27, wherein the polypeptide has acyltransferase activity.

The present inventors have identified a new group of acyltransferases referred to herein as “diacylglycerol acyltransferase-like” or “DGAT2-like” enzymes. Thus, in a preferred embodiment, the present invention provides a substantially purified and/or recombinant polypeptide comprising amino acids having a sequence as provided in any one or more of SEQ ID NOs 29, 102 and 103, a biologically active fragment thereof, or an amino acid sequence which is at least 70% identical to any one or more of SEQ ID NOs 29, 102 and 103, wherein the polypeptide has acyltransferase activity. Preferably, a “DGAT2-like” polypeptide of the invention is more closely related to a DGAT2 polypeptide than other acyltransferases such as those described herein. It is predicted that these enzymes are diacylglycerol acyltransferases, in particular diacylglycerol:diacylglycerol acyltransferases (DDATs). DDAT uses two diacylglycerols to produce a TAG and a free fatty acid.

In another embodiment, the present invention provides a substantially purified and/or recombinant polypeptide comprising amino acids having a sequence as provided in SEQ ID NO:28, a biologically active fragment thereof, or an amino acid sequence which is at least 80% identical to SEQ ID NO:28, wherein the polypeptide has acyltransferase activity.

In another embodiment, the present invention provides a substantially purified and/or recombinant polypeptide comprising amino acids having a sequence as provided in SEQ ID NO:30, a biologically active fragment thereof, or an amino acid sequence which is at least 80% identical to SEQ ID NO:30, wherein the polypeptide has lipase activity.

In another embodiment, the present invention provides a substantially purified and/or recombinant polypeptide comprising amino acids having a sequence as provided in SEQ ID NO:31, a biologically active fragment thereof, or an amino acid sequence which is at least 72% identical to SEQ ID NO:31, wherein the polypeptide has lipase activity.

In another embodiment, the present invention provides a substantially purified and/or recombinant polypeptide comprising amino acids having a sequence as provided in any one or more of SEQ ID NOs 32, 33, 34, 36, 37, 38, 39, 40, 41 and 42, a biologically active fragment thereof, or an amino acid sequence which is at least 30% identical to any one or more of SEQ ID NOs 32, 33, 34, 36, 37, 38, 39, 40, 41 and 42, wherein the polypeptide has lipase activity.

In another embodiment, the present invention provides a substantially purified and/or recombinant polypeptide comprising amino acids having a sequence as provided in SEQ ID NO:35, a biologically active fragment thereof, or an amino acid sequence which is at least 60% identical to SEQ ID NO:35, wherein the polypeptide has lipase activity.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics. Preferred amino acid sequence mutants have only one, two, three, four or less than 10 amino acid changes relative to the reference wildtype polypeptide.

Mutant (altered) polypeptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). Products derived from mutated/altered DNA can readily be screened using techniques described herein to determine if they possess the desired activity such as, but not limited to activity selected from: glycerol-3-phosphate acyltransferase (GPAT), 1-acyl-glycerol-3-phosphate acyltransferase (LPAAT), diacylglycerol acyltransferase (DGAT), acyl-CoA:lysophosphatidylcholine acyltransferase (LPCAT), phospholipase C (PLC), phospholipase D (PLD), CDP-choline diacylglycerol choline phosphotransferase (CPT), phoshatidylcholine diacylglycerol acyltransferase (PDAT), diacylglycerol:diacylglycerol acyltransferase (DDAT) and epoxygenase.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

In a preferred embodiment a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs which are highly conserved between the different polypeptides with the same function provided herewith and/or described in the art. As the skilled person would be aware, such minor changes can reasonably be predicted not to alter the activity of the polypeptide when expressed in a recombinant cell.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as n-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides and Oligonucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the terms “nucleic acid”, “gene” and “mRNA”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over the entire length of their relevant open reading frames.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a preferred embodiment, the present invention provides an isolated and/or exogenous polynucleotide comprising:

(i) a sequence of nucleotides provided as SEQ ID NO:43,

(ii) a sequence of nucleotides encoding a polypeptide of the invention,

(iii) a sequence of nucleotides which is at least 69% identical to the protein coding region of a sequence of nucleotides provided as SEQ ID NO:43, and/or

(iv) a sequence which hybridises to any one of (i) to (iii) under stringent conditions, wherein the polynucleotide encodes a polypeptide with diacylglycerol acyltransferase activity.

In another embodiment, the present invention provides an isolated and/or exogenous polynucleotide comprising:

(i) a sequence of nucleotides provided as SEQ ID NO:44,

(ii) a sequence of nucleotides encoding a polypeptide of the invention,

(iii) a sequence of nucleotides which is at least 65% identical to the protein coding region of a sequence of nucleotides provided as SEQ ID NO:44, and/or

(iv) a sequence which hybridises to any one of (i) to (iii) under stringent conditions, wherein the polynucleotide encodes a polypeptide with diacylglycerol acyltransferase activity.

In another embodiment, the present invention provides an isolated and/or exogenous polynucleotide comprising:

(i) a sequence of nucleotides provided as SEQ ID NO:45,

(ii) a sequence of nucleotides encoding a polypeptide of the invention,

(iii) a sequence of nucleotides which is at least 34% identical to the protein coding region of a sequence of nucleotides provided as SEQ ID NO:45, and/or

(iv) a sequence which hybridises to any one of (i) to (iii) under stringent conditions, wherein the polynucleotide encodes a polypeptide with diacylglycerol acyltransferase activity.

In another embodiment, the present invention provides an isolated and/or exogenous polynucleotide comprising:

(i) a sequence of nucleotides provided as SEQ ID NO:46,

(ii) a sequence of nucleotides encoding a polypeptide of the invention,

(iii) a sequence of nucleotides which is at least 30% identical to the protein coding region of a sequence of nucleotides provided as SEQ ID NO:46, and/or

(iv) a sequence which hybridises to any one of (i) to (iii) under stringent conditions, wherein the polynucleotide encodes a polypeptide with phospholipase A2 activity.

In another embodiment, the present invention provides an isolated and/or exogenous polynucleotide comprising:

(i) a sequence of nucleotides provided as SEQ ID NO:47,

(ii) a sequence of nucleotides encoding a polypeptide of the invention,

(iii) a sequence of nucleotides which is at least 51% identical to the protein coding region of a sequence of nucleotides provided as SEQ ID NO:47, and/or

(iv) a sequence which hybridises to any one of (i) to (iii) under stringent conditions, wherein the polynucleotide encodes a polypeptide with phoshotidylcholine diacylglycerol acyltransferase acyltransferase activity.

In another embodiment, the present invention provides an isolated and/or exogenous polynucleotide comprising:

(i) a sequence of nucleotides provided as SEQ ID NO:48,

(ii) a sequence of nucleotides encoding a polypeptide of the invention,

(iii) a sequence of nucleotides which is at least 77% identical to the protein coding region of a sequence of nucleotides provided as SEQ ID NO:48, and/or

(iv) a sequence which hybridises to any one of (i) to (iii) under stringent conditions, wherein the polynucleotide encodes a polypeptide with phoshotidylcholine diacylglycerol acyltransferase acyltransferase activity.

In another embodiment, the present invention provides an isolated and/or exogenous polynucleotide comprising:

(i) a sequence of nucleotides provided as SEQ ID NO:49,

(ii) a sequence of nucleotides encoding a polypeptide of the invention,

(iii) a sequence of nucleotides which is at least 79% identical to the protein coding region of a sequence of nucleotides provided as SEQ ID NO:49, and/or

(iv) a sequence which hybridises to any one of (i) to (iii) under stringent conditions, wherein the polynucleotide encodes a polypeptide with CDP-choline diacylglycerol choline phosphotransferase activity.

In another embodiment, the present invention provides an isolated and/or exogenous polynucleotide comprising:

(i) a sequence of nucleotides provided as SEQ ID NO:50,

(ii) a sequence of nucleotides encoding a polypeptide of the invention,

(iii) a sequence of nucleotides which is at least 79% identical to the protein coding region of a sequence of nucleotides provided as SEQ ID NO:50, and/or

(iv) a sequence which hybridises to any one of (i) to (iii) under stringent conditions, wherein the polynucleotide encodes a polypeptide with acyl-CoA:lysophosphatidylcholine acyltransferase activity.

In another embodiment, the present invention provides an isolated and/or exogenous polynucleotide comprising:

(i) a sequence of nucleotides provided as SEQ ID NO:51,

(ii) a sequence of nucleotides encoding a polypeptide of the invention,

(iii) a sequence of nucleotides which is at least 75% identical to the protein coding region of a sequence of nucleotides provided as SEQ ID NO:51, and/or

(iv) a sequence which hybridises to any one of (i) to (iii) under stringent conditions, wherein the polynucleotide encodes a polypeptide with acyl-CoA:lysophosphatidylcholine acyltransferase activity.

In another embodiment, the present invention provides an isolated and/or exogenous polynucleotide comprising:

(i) a sequence of nucleotides provided as SEQ ID NO:52,

(ii) a sequence of nucleotides encoding a polypeptide of the invention,

(iii) a sequence of nucleotides which is at least 80% identical to the protein coding region of a sequence of nucleotides provided as SEQ ID NO:52, and/or

(iv) a sequence which hybridises to any one of (i) to (iii) under stringent conditions, wherein the polynucleotide encodes a polypeptide with phospholipase C activity.

In another embodiment, the present invention provides an isolated and/or exogenous polynucleotide comprising:

(i) a sequence of nucleotides provided as SEQ ID NO:53,

(ii) a sequence of nucleotides encoding a polypeptide of the invention,

(iii) a sequence of nucleotides which is at least 66% identical to the protein coding region of a sequence of nucleotides provided as SEQ ID NO:53, and/or

(iv) a sequence which hybridises to any one of (i) to (iii) under stringent conditions, wherein the polynucleotide encodes a polypeptide with phospholipase C activity.

In another embodiment, the present invention provides an isolated and/or exogenous polynucleotide comprising:

(i) a sequence of nucleotides provided as SEQ ID NO:54,

(ii) a sequence of nucleotides encoding a polypeptide of the invention,

(iii) a sequence of nucleotides which is at least 58% identical to the protein coding region of a sequence of nucleotides provided as SEQ ID NO:54, and/or

(iv) a sequence which hybridises to any one of (i) to (iii) under stringent conditions, wherein the polynucleotide encodes a polypeptide with phospholipase C activity.

In another embodiment, the present invention provides an isolated and/or exogenous polynucleotide comprising:

(i) a sequence of nucleotides provided as SEQ ID NO:55,

(ii) a sequence of nucleotides encoding a polypeptide of the invention,

(iii) a sequence of nucleotides which is at least 79% identical to the protein coding region of a sequence of nucleotides provided as SEQ ID NO:55, and/or

(iv) a sequence which hybridises to any one of (i) to (iii) under stringent conditions, wherein the polynucleotide encodes a polypeptide with phospholipase C activity.

In another embodiment, the present invention provides an isolated and/or exogenous polynucleotide comprising:

(i) a sequence of nucleotides provided as SEQ ID NO:56,

(ii) a sequence of nucleotides encoding a polypeptide of the invention,

(iii) a sequence of nucleotides which is at least 92% identical to the protein coding region of a sequence of nucleotides provided as SEQ ID NO:56, and/or

(iv) a sequence which hybridises to any one of (i) to (iii) under stringent conditions, wherein the polynucleotide encodes a polypeptide with phospholipase D activity.

In another embodiment, the present invention provides an isolated and/or exogenous polynucleotide comprising:

(i) a sequence of nucleotides provided as SEQ ID NO:57,

(ii) a sequence of nucleotides encoding a polypeptide of the invention,

(iii) a sequence of nucleotides which is at least 81% identical to the protein coding region of a sequence of nucleotides provided as SEQ ID NO:57, and/or

(iv) a sequence which hybridises to any one of (i) to (iii) under stringent conditions, wherein the polynucleotide encodes a polypeptide with glycerol-3-phosphate acyltransferase activity.

In another embodiment, the present invention provides an isolated and/or exogenous polynucleotide comprising:

(i) a sequence of nucleotides provided as SEQ ID NO:58, 100 or 101,

(ii) a sequence of nucleotides encoding a polypeptide of the invention,

(iii) a sequence of nucleotides which is at least 36% identical to the protein coding region of a sequence of nucleotides provided as one or more of SEQ ID NO:58, 100 or 101, and/or

(iv) a sequence which hybridises to any one of (i) to (iii) under stringent conditions, wherein the polynucleotide encodes a polypeptide with 1-acyl-glycerol-3-phosphate acyltransferase activity.

In another embodiment, the present invention provides an isolated and/or exogenous polynucleotide comprising:

(i) a sequence of nucleotides provided as SEQ ID NO:59,

(ii) a sequence of nucleotides encoding a polypeptide of the invention,

(iii) a sequence of nucleotides which is at least 58% identical to the protein coding region of a sequence of nucleotides provided as SEQ ID NO:59, and/or

(iv) a sequence which hybridises to any one of (i) to (iii) under stringent conditions, wherein the polynucleotide encodes a polypeptide with acyltransferase activity.

In another embodiment, the present invention provides an isolated and/or exogenous polynucleotide comprising:

(i) a sequence of nucleotides provided as SEQ ID NO:60,

(ii) a sequence of nucleotides encoding a polypeptide of the invention,

(iii) a sequence of nucleotides which is at least 75% identical to the protein coding region of a sequence of nucleotides provided as SEQ ID NO:60, and/or

(iv) a sequence which hybridises to any one of (i) to (iii) under stringent conditions, wherein the polynucleotide encodes a polypeptide with acyltransferase activity.

In another embodiment, the present invention provides an isolated and/or exogenous polynucleotide comprising:

(i) a sequence of nucleotides provided as SEQ ID NO:61,

(ii) a sequence of nucleotides encoding a polypeptide of the invention,

(iii) a sequence of nucleotides which is at least 89% identical to the protein coding region of a sequence of nucleotides provided as SEQ ID NO:61, and/or

(iv) a sequence which hybridises to any one of (i) to (iii) under stringent conditions, wherein the polynucleotide encodes a polypeptide with acyltransferase activity.

In another embodiment, the present invention provides an isolated and/or exogenous polynucleotide comprising:

(i) a sequence of nucleotides provided as SEQ ID NO:62,

(ii) a sequence of nucleotides encoding a polypeptide of the invention,

(iii) a sequence of nucleotides which is at least 58% identical to the protein coding region of a sequence of nucleotides provided as SEQ ID NO:62, and/or

(iv) a sequence which hybridises to any one of (i) to (iii) under stringent conditions, wherein the polynucleotide encodes a polypeptide with acyltransferase activity.

In another embodiment, the present invention provides an isolated and/or exogenous polynucleotide comprising:

(i) a sequence of nucleotides provided as SEQ ID NO:63,

(ii) a sequence of nucleotides encoding a polypeptide of the invention,

(iii) a sequence of nucleotides which is at least 34% identical to the protein coding region of a sequence of nucleotides provided as SEQ ID NO:63, and/or

(iv) a sequence which hybridises to any one of (i) to (iii) under stringent conditions, wherein the polynucleotide encodes a polypeptide with fatty acid epoxygenase activity.

In another embodiment, the present invention provides an isolated and/or exogenous polynucleotide comprising:

(i) a sequence of nucleotides provided as SEQ ID NO:64,

(ii) a sequence of nucleotides encoding a polypeptide of the invention,

(iii) a sequence of nucleotides which is at least 58% identical to the protein coding region of a sequence of nucleotides provided as SEQ ID NO:64, and/or

(iv) a sequence which hybridises to any one of (i) to (iii) under stringent conditions, wherein the polynucleotide encodes a polypeptide with M2 destaurase activity.

In another embodiment, the present invention provides an isolated and/or exogenous polynucleotide comprising:

(i) a sequence of nucleotides provided as SEQ ID NO:65,

(ii) a sequence of nucleotides encoding a polypeptide of the invention,

(iii) a sequence of nucleotides which is at least 74% identical to the protein coding region of a sequence of nucleotides provided as SEQ ID NO:65, and/or

(iv) a sequence which hybridises to any one of (i) to (iii) under stringent conditions, wherein the polynucleotide encodes a polypeptide with fatty acid modifying activity.

In another embodiment, the present invention provides an isolated and/or exogenous polynucleotide comprising:

(i) a sequence of nucleotides provided as SEQ ID NO:66,

(ii) a sequence of nucleotides encoding a polypeptide of the invention,

(iii) a sequence of nucleotides which is at least 79% identical to the protein coding region of a sequence of nucleotides provided as SEQ ID NO:66, and/or

(iv) a sequence which hybridises to any one of (i) to (iii) under stringent conditions, wherein the polynucleotide encodes a polypeptide with fatty acid modifying activity.

In another embodiment, the present invention provides an isolated and/or exogenous polynucleotide comprising:

(i) a sequence of nucleotides provided as any one or more of SEQ ID NOs 67, 68 and 69,

(ii) a sequence of nucleotides encoding a polypeptide of the invention,

(iii) a sequence of nucleotides which is at least 30% identical to the protein coding region of a sequence of nucleotides provided as any one or more of SEQ ID NOs 67, 68 and 69, and/or

(iv) a sequence which hybridises to any one of (i) to (iii) under stringent conditions, wherein the polynucleotide encodes a polypeptide with acyltransferase activity.

As outlined above, the present inventors have identified a new group of acyltransferases referred to herein as “diacylglycerol acyltransferase-like” or

“DGAT2-like” enzymes. Thus, in a preferred embodiment, the present invention provides an isolated and/or exogenous polynucleotide comprising:

(i) a sequence of nucleotides provided as any one or more of SEQ ID NOs 71, 104 and 105,

(ii) a sequence of nucleotides encoding a polypeptide of the invention,

(iii) a sequence of nucleotides which is at least 30% identical to the protein coding region of a sequence of nucleotides provided as any one or more of SEQ ID NOs 71, 104 and 105, and/or

(iv) a sequence which hybridises to any one of (i) to (iii) under stringent conditions, wherein the polynucleotide encodes a polypeptide with acyltransferase activity, preferably diacylglycerol acyltransferase activity, more preferably diacylglycerol:diacylglycerol acyltransferase (DDAT) activity.

In another embodiment, the present invention provides an isolated and/or exogenous polynucleotide comprising:

(i) a sequence of nucleotides provided as SEQ ID NO:70,

(ii) a sequence of nucleotides encoding a polypeptide of the invention,

(iii) a sequence of nucleotides which is at least 80% identical to the protein coding region of a sequence of nucleotides provided as SEQ ID NO:70, and/or

(iv) a sequence which hybridises to any one of (i) to (iii) under stringent conditions, wherein the polynucleotide encodes a polypeptide with acyltransferase activity.

In another embodiment, the present invention provides an isolated and/or exogenous polynucleotide comprising:

(i) a sequence of nucleotides provided as SEQ ID NO:72,

(ii) a sequence of nucleotides encoding a polypeptide of the invention,

(iii) a sequence of nucleotides which is at least 80% identical to the protein coding region of a sequence of nucleotides provided as SEQ ID NO:72, and/or

(iv) a sequence which hybridises to any one of (i) to (iii) under stringent conditions, wherein the polynucleotide encodes a polypeptide with lipase activity.

In another embodiment, the present invention provides an isolated and/or exogenous polynucleotide comprising:

(i) a sequence of nucleotides provided as SEQ ID NO:74,

(ii) a sequence of nucleotides encoding a polypeptide of the invention,

(iii) a sequence of nucleotides which is at least 74% identical to the protein coding region of a sequence of nucleotides provided as SEQ ID NO:73, and/or

(iv) a sequence which hybridises to any one of (i) to (iii) under stringent conditions, wherein the polynucleotide encodes a polypeptide with lipase activity.

In another embodiment, the present invention provides an isolated and/or exogenous polynucleotide comprising:

(i) a sequence of nucleotides provided as any one or more of SEQ ID NOs 75, 76, 77, 79, 80, 81, 82, 83, 84 and 85,

(ii) a sequence of nucleotides encoding a polypeptide of the invention,

(iii) a sequence of nucleotides which is at least 79% identical to the protein coding region of a sequence of nucleotides provided as any one or more of SEQ ID NOs 75, 76, 77, 79, 80, 81, 82, 83, 84 and 85, and/or

(iv) a sequence which hybridises to any one of (i) to (iii) under stringent conditions, wherein the polynucleotide encodes a polypeptide with lipase activity.

In another embodiment, the present invention provides an isolated and/or exogenous polynucleotide comprising:

(i) a sequence of nucleotides provided as SEQ ID NO:78,

(ii) a sequence of nucleotides encoding a polypeptide of the invention,

(iii) a sequence of nucleotides which is at least 60% identical to the protein coding region of a sequence of nucleotides provided as SEQ ID NO:78, and/or

(iv) a sequence which hybridises to any one of (i) to (iii) under stringent conditions, wherein the polynucleotide encodes a polypeptide with lipase activity.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

Oligonucleotides of the present invention can be RNA, DNA, or derivatives of either. The minimum size of such oligonucleotides is the size required for the formation of a stable hybrid between an oligonucleotide and a complementary sequence on a nucleic acid molecule of the present invention. Preferably, the oligonucleotides are at least 15 nucleotides, more preferably at least 18 nucleotides, more preferably at least 19 nucleotides, more preferably at least 20 nucleotides, even more preferably at least 25 nucleotides in length. The present invention includes oligonucleotides that can be used as, for example, probes to identify nucleic acid molecules, or primers to produce nucleic acid molecules. Oligonucleotide of the present invention used as a probe are typically conjugated with a label such as a radioisotope, an enzyme, biotin, a fluorescent molecule or a chemiluminescent molecule.

Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to a sequence provided as SEQ ID NO's: 43 to 85, 100, 101, 104 or 105. As used herein, stringent conditions are those that (1) employ low ionic strength and high temperature for washing, for example, 0.015 M NaCl/0.0015 M sodium citrate/0.1% NaDodSO₄ at 60° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Usually, monomers of a polynucleotide or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate, phosphoramidate.

### Antisense Polynucleotides

The term “antisense polynucleotide” shall be taken to mean a DNA or RNA, or combination thereof, molecule that is complementary to at least a portion of a specific mRNA molecule encoding a polypeptide defined herein and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque, 1995 and Senior, 1998. Bourque, 1995 lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior (1998) states that antisense methods are now a very well established technique for manipulating gene expression.

An antisense polynucleotide of the invention will hybridize to a target polynucleotide under physiological conditions. As used herein, the term “an antisense polynucleotide which hybridises under physiological conditions” means that the polynucleotide (which is fully or partially single stranded) is at least capable of forming a double stranded polynucleotide with mRNA encoding a protein under normal conditions in a cell, preferably a plant cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988; Perriman et al., 1992) and the hairpin ribozyme (Shippy et al., 1999).

The ribozymes of this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. Accordingly, also provided by this invention is a nucleic acid molecule, i.e., DNA or cDNA, coding for a catalytic polynucleotide of the invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides of the invention should also be capable of hybridizing a target nucleic acid molecule under “physiological conditions”, namely those conditions within a cell (especially conditions in a plant cell).

### RNA Interference

The terms “RNA interference”, “RNAi” or “gene silencing” refers generally to a process in which a double-stranded RNA molecule reduces the expression of a nucleic acid sequence with which the double-stranded RNA molecule shares substantial or total homology. However, it has more recently been shown that RNA interference can be achieved using non-RNA double stranded molecules (see, for example, US 20070004667).

RNA interference (RNAi) is particularly useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous plant system that destroys both the double stranded RNA and also the homologous RNA transcript from the target plant gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

microRNA

MicroRNA regulation is a clearly specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005). In an embodiment, the microRNA has 21 consecutive nucleotides of which at least 20 nucleotides, preferably all 21 nucleotides, are identical in sequence to the complement of 21 consecutive nucleotides of the transcribed region of the target gene. That is, the microRNA can tolerate 1 mismatched nucleotide in the sequence of 21 nucleotides, but preferably is identical to the complement of the region of the target gene. The remainder of the stern-looped precursor RNA to the microRNA may be unrelated in sequence to the target gene, and is preferably related in sequence to, or corresponds to, a naturally occurring microRNA precursor.

### Cosuppression

Another molecular biological approach that may be used is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the sense orientation with respect to a promoter for its expression. The size of the sense fragment, its correspondence to target gene regions, and its degree of sequence identity to the target gene are as for the antisense sequences described above. In some instances the additional copy of the gene sequence interferes with the expression of the target plant gene. Reference is made to WO 97/20936 and EP 0465572 for methods of implementing co-suppression approaches.

### Gene Constructs and Vectors

One embodiment of the present invention includes a recombinant (chimeric) vector, which includes at least one isolated polynucleotide molecule encoding a polypeptide/enzyme defined herein, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

One type of recombinant vector comprises a nucleic acid molecule of the present invention operatively linked to an expression vector. As indicated above, the phrase operatively linked refers to insertion of a nucleic acid molecule into an expression vector in a manner such that the molecule is able to be expressed when transformed into a host cell. As used herein, an expression vector is a DNA or RNA vector that is capable of transforming a host cell and effecting expression of a specified nucleic acid molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, other animal, and plant cells. Preferred expression vectors of the present invention can direct gene expression in yeast, or plant cells.

In particular, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art.

Another embodiment of the present invention includes a recombinant cell comprising a host cell transformed with one or more recombinant molecules of the present invention. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules can remain extrachromosomal or can integrate into one or more sites within a chromosome of the transformed (i.e., recombinant) cell in such a manner that their ability to be expressed is retained.

### Transgenic Plants and Parts Thereof

The term “plant” as used herein as a noun refers to whole plants, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plants provided by or contemplated for use in the practice of the present invention include both monocotyledons and dicotyledons. In preferred embodiments, the plants of the present invention are crop plants (for example, cereals and pulses, maize, wheat, potatoes, tapioca, rice, sorghum, millet, cassaya, barley, or pea), or other legumes. The plants may be grown for production of edible roots, tubers, leaves, stems, flowers or fruit. The plants may be vegetables or ornamental plants. The plants of the invention may be: corn (Zea mays), canola (Brassica napus, Brassica rapa ssp.), flax (Linum usitatissimum), alfalfa (Medicago sativa), rice (Oryza sativa), rye (Secale cerale), sorghum (Sorghum bicolour, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Lopmoea batatus), cassaya (Manihot esculenta), coffee (Cofea spp.), coconut (Cocos nucifera), pineapple (Anana comosus), citris tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, or barley.

Grain plants that provide seeds of interest include oil-seed plants and leguminous plants. Seeds of interest include grain seeds, such as corn, wheat, barley, rice, sorghum, rye, etc. Leguminous plants include beans and peas. Beans include guar, locust bean, fenugreek, soybean, garden beans, cowpea, mungbean, lima bean, fava bean, lentils, chickpea, etc.

In one embodiment, the plant is an oilseed plant, preferably an oilseed crop plant. As used herein, an “oilseed plant” is a plant species used for the commercial production of oils from the seeds of the plant. The plant may produce high levels of oil in its fruit, such as olive, oil palm or coconut. Preferably, the oilseed plant is Brassica sp., Gossypium hirsutum, Linum usitatissimum, Helianthus sp., Carthamus tinctorius, Glycine max, Zea mays or Arabidopsis thaliana. More preferably, the oilseed plant is Linum usitatissimum or Carthamus tinctorius.

Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the transgenic plants are homozygous for each and every exogenous polynucleotide that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

In addition to other transgenes already mentioned, the transgenic plants may also comprise further transgenes involved in the production of LC-PUFAs such as, but not limited to, a Δ6 desaturase, a Δ9 elongase, a Δ8 desaturase, a Δ6 elongase, a Δ5 desaturase with activity on a 20:3 substrate, an omega-desaturase, a Δ9 elongase, a Δ4 desaturase, a Δ7 elongase and/or members of the polyketide synthase pathway. Examples of such enzymes are known in the art and include those described in WO 05/103253 (see, for example, Table 1 of WO 05/103253).

The polynucleotide(s) may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants; see, e.g., PCT publication WO 8402913. All of these promoters have been used to create various types of plant-expressible recombinant DNA vectors.

For the purpose of expression in tissues of the plant such as seed, particularly seed of an oilseed plant such as of soybean, canola, other Brassicas, cotton, Zea mays, sunflower, safflower, or flax, it is preferred that the promoters utilized in the present invention have relatively high expression in the seed before and/or during production of fatty acids for accumulation and storage in the seed. The promoter for (3-conglycinin or other seed-specific promoters such as the linin, napin and phaseolin promoters, can be used.

In a preferred embodiment, the promoter directs expression in tissues and organs in which fatty acid and oil biosynthesis take place, particularly in seed cells such as endosperm cells and cells of the developing embryo. Promoters which are suitable are the oilseed rape napin gene promoter (U.S. Pat. No. 5,608,152), the Vicia faba USP promoter (Baumlein et al., 1991), the Arabidopsis oleosin promoter (WO 98/45461), the Phaseolus vulgaris phaseolin promoter (U.S. Pat. No. 5,504,200), the Brassica Bce4 promoter (WO 91/13980) or the legumin B4 promoter (Baumlein et al., 1992), and promoters which lead to the seed-specific expression in monocots such as maize, barley, wheat, rye, rice and the like. Notable promoters which are suitable are the barley 1pt2 or 1pt1 gene promoter (WO 95/15389 and WO 95/23230) or the promoters described in WO 99/16890. Other promoters include those described by Broun et al. (1998) and US 20030159173.

The 5′ non-translated leader sequence can be derived from the promoter selected to express the heterologous gene sequence of the polynucleotide of the present invention, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. No. 5,362,865 and U.S. Pat. No. 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. No. 4,945,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. An illustrative embodiment of a method for delivering DNA into Zea mays cells by acceleration is a biolistics α-particle delivery system, that can be used to propel particles coated with DNA through a screen, such as a stainless steel or Nytex screen, onto a filter surface covered with corn cells cultured in suspension. A particle delivery system suitable for use with the present invention is the helium acceleration PDS-1000/He gun available from Bio-Rad Laboratories.

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus that express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include particle gun delivery of DNA containing a selectable marker and targeting of the DNA to the plastid genome through homologous recombination (U.S. Pat. No. 5,451,513, U.S. Pat. No. 5,545,818, U.S. Pat. No. 5,877,402, U.S. Pat. No. 5,932,479, and WO 99/05265).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and that may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. No. 5,177,010, U.S. Pat. No. 5,104,310, U.S. Pat. No. 5,004,863, U.S. Pat. No. 5,159,135). Further, the integration of the T-DNA is a relatively precise process resulting in few rearrangements. The region of DNA to be transferred is defined by the border sequences, and intervening DNA is usually inserted into the plant genome.

Modern Agrobacterium transformation vectors are capable of replication in E. coli as well as Agrobacterium, allowing for convenient manipulations as described (Klee et al., In: Plant DNA Infectious Agents, Hohn and Schell, eds., Springer-Verlag, New York, pp. 179-203 (1985). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added gene; i.e., a transgenic plant that contains two added genes, one gene at the same locus on each chromosome of a chromosome pair. A homozygous transgenic plant can be obtained by sexually mating (selling) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, In: Breeding Methods for Cultivar Development, Wilcox J. ed., American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, Canadian Patent Application No. 2,092,588, Australian Patent Application No 61781/94, Australian Patent No 667939, U.S. Pat. No. 6,100,447, International Patent Application PCT/US97/10621, U.S. Pat. No. 5,589,617, U.S. Pat. No. 6,541,257, and other methods are set out in Patent specification WO99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts.

The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (Ed. M. J. McPherson and S. G Moller (2000) BIOS Scientific Publishers Ltd, Oxford). PCR can be performed on cDNA obtained from reverse transcribing mRNA isolated from plant cells. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al. (supra) and Sambrook et al. (supra). Sequencing can be carried out by any suitable method, for example, dideoxy sequencing, chemical sequencing or variations thereof. Direct sequencing has the advantage of determining variation in any base pair of a particular sequence.

### Production of Oils

Techniques that are routinely practiced in the art can be used to extract, process, and analyze the oils produced by cells, plants, seeds, etc of the instant invention. Typically, plant seeds are cooked, pressed, and extracted to produce crude oil, which is then degummed, refined, bleached, and deodorized. Generally, techniques for crushing seed are known in the art. For example, oilseeds can be tempered by spraying them with water to raise the moisture content to, e.g., 8.5%, and flaked using a smooth roller with a gap setting of 0.23 to 0.27 mm. Depending on the type of seed, water may not be added prior to crushing. Application of heat deactivates enzymes, facilitates further cell rupturing, coalesces the oil droplets, and agglomerates protein particles, all of which facilitate the extraction process.

The majority of the seed oil is released by passage through a screw press. Cakes expelled from the screw press are then solvent extracted, e.g., with hexane, using a heat traced column. Alternatively, crude oil produced by the pressing operation can be passed through a settling tank with a slotted wire drainage top to remove the solids that are expressed with the oil during the pressing operation. The clarified oil can be passed through a plate and frame filter to remove any remaining fine solid particles. If desired, the oil recovered from the extraction process can be combined with the clarified oil to produce a blended crude oil.

Once the solvent is stripped from the crude oil, the pressed and extracted portions are combined and subjected to normal oil processing procedures (i.e., degumming, caustic refining, bleaching, and deodorization). Degumming can be performed by addition of concentrated phosphoric acid to the crude oil to convert non-hydratable phosphatides to a hydratable form, and to chelate minor metals that are present. Gum is separated from the oil by centrifugation. The oil can be refined by addition of a sufficient amount of a sodium hydroxide solution to titrate all of the fatty acids and removing the soaps thus formed.

Deodorization can be performed by heating the oil to 260° C. under vacuum, and slowly introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. If the amount of oil is limited, the oil can be placed under vacuum, e.g., in a Parr reactor and heated to 260° C. for the same length of time that it would have been deodorized. This treatment improves the color of the oil and removes a majority of the volatile substances.

### Antibodies

The invention also provides antibodies, such as monoclonal or polyclonal antibodies, to polypeptides of the invention or fragments thereof. Thus, the present invention further provides a process for the production of monoclonal or polyclonal antibodies to polypeptides of the invention.

The term “binds specifically” refers to the ability of the antibody to bind to at least one protein of the present invention but not other proteins present in a recombinant (transgenic) cell, particularly a recombinant plant cell of the invention.

As used herein, the term “epitope” refers to a region of a protein of the invention which is bound by the antibody. An epitope can be administered to an animal to generate antibodies against the epitope, however, antibodies of the present invention preferably specifically bind the epitope region in the context of the entire protein.

If polyclonal antibodies are desired, a selected mammal (e.g., mouse, rabbit, goat, horse, etc.) is immunised with an immunogenic polypeptide. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. Monoclonal antibodies directed against polypeptides of the invention can also be readily produced by one skilled in the art. The general methodology for making monoclonal antibodies by hybridomas is well known.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv).

### EXAMPLES

### Example 1

### Materials and Methods

### Developing Embryos

Seed of Bernardia pulchella, a dioecious Euphorbia species containing 90% vernolic acid in its seeds, were obtained from Belgium Botanical Gardens and used to establish plants in the glasshouse. Flowers on male and female plants were intercrossed using brush pollination techniques. Green developing embryos were harvested at a range of different growth stages as described below.

Construction of Bernardia pulchella cDNA Library

Total RNA was isolated from developing seeds ranging 4-8 mm in size using Trizol reagent (Invitrogen) according to the instructions of the supplier. Messenger RNA was purified from total RNA using an Oligotex mRNA kit (Qiagen). First strand cDNA was synthesised from 5 μg mRNA using an oligo-dT primer supplied with the λ ZAP II-cDNA synthesis kit (Stratagene—Catalogue No. 200400) and reverse transcriptase SuperscriptIII (Invitrogen). Double stranded cDNA was ligated to EcoRI/XhoI adaptors and from this a library was constructed using the λ ZAP II-cDNA synthesis kit according to the suppliers' instructions. The titer of the primary library was 4×10⁶ plaque forming units (pfu)/ml and that of the amplified library was 3×10⁹ pfu/ml. The average insert size of cDNA inserts in the library was 1.4 kilobases and the percentage of recombinants in the library was 96%.

Bulk Excision and EST Sequencing of B. pulchella cDNA Library

A portion of the unamplified cDNA library containing 3×10⁴ pfu was excised from the viral vectors into plasmids in colonies by infecting 100 μL of 10 mM MgSO₄ pretreated XL-1 Blue MRF′ cells (Stratagene) at OD₆₀₀=1.0, and 10 μL of ExAssist helper phage (1×10⁸ pfu, Stratagene). After infection at 37° C. for 15 mins, 1.5 mL of 37° C. pre-warmed LB medium was added, and the mixture incubated at 37° C. for 2 hours. The mixture was heated to 65° C. for 20 min, and phagemid supernatant recovered after centrifuging at 14,000 rpm for 5 mins. The phagemid was used to infect 10 mM MgSO₄ pretreated SOLR cells (Stratagene) at OD₆₀₀=1.0 (100 μL of cells for each 50 μL phagemid) for 15 mins, then incubated at 37° C. for 45 mins after added 300 μL of 37° C. pre-warmed LB media. The cells were then collected by centrifuging, and plated out on LB/ampicillin/IPTG/X-gal plates, until enough colonies were obtained for EST sequencing. White colonies were selected for plasmid DNA extraction and sequenced with standard Reverse primer (Beijing Genomic Institute, Beijing, China). The resultant sequences were translated to obtain predicted amino acid sequences which were used to search for homologous sequences in GenBank database by BlastX.

B. pulchella cDNA Library Screening

XL1-Blue MRF′ cells were grown in LB broth with 10 mM MgSO₄ and 0.2% maltose at 30° C. overnight, collected by centrifuging 1000×g, and resuspended in 10 mM MgSO₄ at OD₆₀₀ of 0.5. An aliquot of the B. pulchella cDNA library (5×10⁵ pfu) was added to the XL1-Blue MRF′ cells at 37° C. for 15 min, and mixed with NZY top agar for plating out. The resultant phage plaques were then lifted to Hybond N⁺ membranes, which were then denatured with 1.5 M NaCl/0.5M NaOH, then neutralized with 1.5 M NaCl/0.5M Tris-HCl (pH8.0), and finally rinsed with 2×SSC buffer. After air drying, the membranes were hybridized with radioactively-labelled probes at 60° C. overnight and washed with 2×SSC/0.1% SDS for 30 min at 60° C., followed by washing with 0.2×SSC/0.1% SDS for 30 min at 60° C. for high stringency; or 55° C. overnight and washed at 60° C. with 2×SSC/0.1% SDS three times each for 10 minutes for moderate stringency. The plasmids were excised from the positive plaques, and the nucleotide sequences of the inserts were determined.

### Construction of Expression Plasmids

B. pulchella protein coding regions or gene fragments in selected cDNA clones were cut out of the vectors with restriction enzymes and ligated to similar digested pENTR11 entry vector (Invitrogen), and transformed into E. coli DH5α. Kanamycin resistant/ampicillin sensitive colonies were selected and inserts in the plasmids sequenced to confirm their identity, and then recombined using LR Clonase (Invitrogen) into the yeast vector pYES-DEST52 (Invitrogen) for yeast expression or into pXZP391 for plant expression under control of the Fp1 seed specific promoter (Stalberg et al., 1993). The resulted yeast expression plasmids were transformed into yeast strain S288C or other strains as described below, some of which were mutant in selected genes for complementation analysis. The resulted plant expression plasmids were transformed into Agrobacterium tumefaciens strain AGL1 and used for plant transformation by standard methods.

Yeast Culturing and Feeding with Precursor Fatty Acids

Plasmids were introduced into yeast by a standard heat shock method and transformants selected on yeast synthetic drop out (SD) medium plates containing 2% glucose or raffinose as the sole carbon source. Cultures for use as inoculae were established in liquid yeast minimal media (YMM) with 2% glucose or raffinose as the sole carbon source. Experimental cultures were inoculated from these in YMM medium containing 1% NP-40, to an initial OD₆₀₀ of about 0.3. Cultures were grown at 30° C. with shaking until OD₆₀₀ was approximately 1.0. The cells were harvested by centrifugation and washed with sterile water, then resuspended into the same volume of synthetic media with 2% galactose (SG) instead of glucose. Selected precursor fatty acids were added to a final concentration of 0.5 mM at the presence of 1% NP-40. Cultures were incubated at 30° C. with shaking for a further 48 hours prior to harvesting by centrifugation. Cell pellets were washed with 1% NP-40, 0.5% NP-40 and water to remove any unincorporated fatty acids from the surface of the cells.

### Plant Transformation

Arabidopsis thaliana transgenic lines Ven9 and BU18 expressing Crepis palaestina Δ12-epoxygenase gene Cpal2 were used in transformation experiments. Ven9 was a Cpal2 homozygous T₃ plant from the AO*10 line in the A. thaliana C24 ecotype (Singh et al., 2001) and producing about 7% (mol %) vernolic acid in seed oil. These plants also exhibited a reduced oleic acid desaturation level in the seed oil compared to wild-type plants of the C24 genotype. BU18 was a T3 line homozygous for the exogenous Cpal2 gene expressed from an Fp1 promoter, and also was homozygous for both fad3 and fae1 alleles which inactivate the FAD3 gene encoding Δ15 desaturase and FAE1 encoding a fatty acid elongase, and in addition was transformed with a C. palaestina Δ12-desaturase gene Cpdes (Zhou et al., 2006). Seed oil of BU18 contained up to 21% vernolic acid as a percentage of total fatty acid in the seed oil, with an oleic acid desaturation level the same as wild-type.

Arabidopsis transformations were done by spraying flower buds with suspensions of A. tumefaciens (AGL1 strain) carrying the various expression constructs made as described above. Seeds were collected from the treated plants (T₀ generation) at maturity. Primary transformants (T₁ generation) were identified by plating the seeds on medium containing kanamycin, where expression of antibiotic resistance was indicative of presence of the Kan selectable marker gene and therefore of transformation (Stoutjesdijk et al., 2002). All transgenic Arabidopsis plants were grown in a greenhouse under natural day-length at controlled temperatures of 24° C. in the daylight hours and 18° C. during the night. Selfed seeds (T₂ generation) from the T₁ plants were harvested and the seed fatty acid composition was analysed by gas-liquid chromatography (GC) by standard methods. For segregation studies, individual T₂ seeds were planted, the T₂ plants grown to maturity, and T₃ seeds were harvested and analysed for antibiotic resistance and fatty acid composition of seed oil by GC.

### Fatty Acid Methyl Esters (FAME) Preparation

Fatty acid methyl esters (FAME) were formed by transesterification of the total fatty acids in yeast cells, obtained as cell pellets after centrifugation of cultures, or Arabidopsis seeds by adding 300 μL of 1% NaMeOH in methanol at room temperature for 20 min, then added 300 μL of 1M NaCl. FAMEs were extracted with 300 uL of hexane and analysed by GC and GC-MS.

### Capillary Gas-Liquid Chromatography (GC)

FAME were analysed with an Agilent 6890 gas chromatograph fitted with 6980 series automatic injectors respectively and a flame-ionization detector (FID). Injector and detector temperatures used were 240° C. and 280° C. respectively. FAME samples were injected at 170° C. onto a BPX70 polar capillary column (SGE; 60 m×0.25 mm i.d.; 0.25 μm film thickness). After 2 min, the oven temperature was raised to 200° C. at 5° C. min⁻¹, to 210° C. at 2.5° C. min⁻¹, then to a final temperature of 240° C. at 10° C. min⁻¹ where it was kept for 4 min. Helium was the carrier gas with a column head pressure of 45 psi and the purge opened 2 min after injection. Identification of peaks was based on comparison of relative retention time data with standard FAMEs. For quantification, Chemstation (Agilent) was used to integrate peak areas.

### Gas Chromatography-Mass Spectrometry (GC-MS)

GC-MS was carried out on a Finnigan Polaris Q and Trace GC2000 GC-MS ion-trap fitted with on-column injection. Samples were injected using an AS3000 auto sampler onto a retention gap attached to a BPX70 polar capillary column (SGE; 30 m×0.25 mm i.d.; 0.25 μm film thickness). The initial temperature of 60° C. was held for 1 min, followed by temperature programming at 30° C.min⁻¹ to 120° C. then at 9° C.min⁻¹ to 250° C. where it was held for 1 min. Helium was used as the carrier gas. Mass spectra were acquired and processed with Xcalibur™ software.

### Example 2

### Isolation and Expression of B. pulchella Diacylglycerol Acyltransferase 2 (BpDGAT2)

Acyl CoA:diacylglycerol acyltransferase (EC 2.3.1.20; DGAT) catalyzes the final step in TAG assembly by transferring a fatty acyl group from acyl-CoA to a diacylglycerol substrate. Three different, structurally unrelated DGAT enzymes have been identified in plants. Since they have the same enzyme activity, they are isoenzymes. The first two to be identified were DGAT1 and DGAT2, both of which were endoplasmic reticulum (ER)-localized and contained predicted membrane spanning domains (Hobbs et al., 2000; Zou et al. 1999; Lardizabal et al., 2001). The third enzyme was a soluble DGAT (DGAT3), which was recently identified in peanut (Saha et al., 2006) but has not been characterized in other species.

Although type 2 diacylglycerol acyltransferase genes (DGAT2) encode proteins with DGAT activity, they are unrelated in amino acid sequence to proteins encoded by DGAT1 gene family as determined by BLAST analysis. Gene disruption of DGAT1 in Arabidopsis did not abolish DGAT activity completely. DGAT2 protein was smaller than DGAT1, and located in different dynamic regions of the endoplasmic reticulum (Shockey et al., 2006). DGAT2 was predicted to have only 2 transmembrane domains, compared to the 10 transmembrane domains predicted in DGAT1.

### Cloning of BpDGAT2 by EST Sequencing and Library Screening

A total of 12,180 clones of the B. pulchella cDNA library (Example 1) were sequenced from the 5′ end. The amino acid sequences predicted from the nucleotide sequences were screened for protein sequences homologous to Arabidopsis AtDGAT1 (At2g19450) and AtDGAT2 (At3g51520), Ricinus communis DGAT2 (AAY16324) and Vernicia fordii VfDGAT2 (ABC94474), but different to BpDGAT1 (see Example 3). Five DGAT2-like sequences were identified from the 12,180 EST sequences, namely cDNA clones Bp201685, Bp209844, Bp211489, Bp211518 and Bp212233. After completing the sequence analysis of the cDNA insert, Bp209844 was predicted to contain a full-length cDNA (SEQ ID NO:43), while Bp201685 Bp211489, Bp211518 and Bp212233 were partial length cDNA clones.

The open reading frame encoding the DGAT2 protein started with the ATG start codon at nucleotides 232-234 and was terminated by the TGA stop codon at nucleotides 1210-1212. The deduced amino acid sequence of the gene in Bp209844 is shown in SEQ ID NO:1. The sequence of 326 amino acids showed 58%, 68% and 66% identity to AtDGAT2 (At3g51520), RcDGAT (AAY16324) and VfDGAT2 (ABC94474), respectively. Scanning the BpDGAT2 protein sequence against the Prosite database (http://expasy.org/tools/scanprosite) identified at least one potential N-linked glycosylation site (residues 173-176; -NFTS-), three potential protein kinase C phosphorylation sites (residues 110-112, 170-172 and 208-210), one casein kinase II phosphorylation site, and four N-myristoylation sites (residues 81-86, 165-170, 190-195, 200-205).

### Expression of BpDGAT2

The full-length BpDGAT2 cDNA was cloned into pENTR11 as an EcoRI-XhoI fragment to generate entry plasmid pXZP080E. The gene was then recombined into pYES-DEST52 and pXZP391 by LR Clonase, resulting in plasmids pXZP238 pXZP378, respectively. The DGAT function and substrate specificity of the gene expressed in transformed yeast cells is analyzed as described in Example 1.

Twenty-one and eleven transgenic FG and FC lines were generated with pXZP378 in Ven9 and BU18, respectively. The vernolic acid levels and oleic desaturation proportion (ODP) of transgenic seeds from these lines were shown in Table 2. ODP represents the “oleic desaturation proportion”, which is the ratio of the amount of desaturated fatty acids derived from C18:1 to the sum of the amounts of the remaining C18:1 and the desaturated fatty acids derived from C18:1.

The vernolic acid levels in seed oil of plants expressing DGAT2 in the Ven9 background ranged from similar to Ven9 to 13.3%, while in the BU18 background levels of 28% were observed in some lines compared to about around 20% for BU18 without the DGAT2 transgene, suggesting an enhancing effect of DGAT2 on accumulation of vernolic acid.

TABLE 2
Seed oil composition of Arabidopsis Ven9 and BU18 and transgenic derivatives carrying the BpDGAT2 gene.

| | | | | | | | | | | | Total | |
|---|---|---|---|---|---|---|---|---|---|---|---|---|
| Plant | C16:0 | C18:0 | C18:1 | C18:1n7 | C18:2 | C20:0 | C18:3 | C20:1 | Ver | C18:2E | Epoxy | ODP |
| Ven9 | 5.0 | 3.4 | 32.2 | 1.1 | 8.2 | 1.2 | 17.9 | 18.8 | 7.0 | 3.4 | 10.4 | 0.53 |
| FG1 | 5.3 | 2.7 | 28.0 | 1.2 | 11.2 | 1.1 | 19.3 | 18.0 | 9.3 | 4.0 | 13.3 | 0.61 |
| FG4 | 5.3 | 2.7 | 27.4 | 1.0 | 11.5 | 1.2 | 20.6 | 18.5 | 8.2 | 3.5 | 11.7 | 0.62 |
| FG5 | 5.4 | 2.9 | 33.4 | 0.0 | 12.2 | 0.0 | 19.6 | 17.7 | 8.9 | 0.0 | 8.9 | 0.55 |
| FG6 | 5.2 | 2.6 | 30.9 | 0.0 | 10.9 | 1.1 | 18.6 | 18.9 | 8.3 | 3.5 | 11.8 | 0.57 |
| FG7 | 5.4 | 2.9 | 25.0 | 0.0 | 16.3 | 1.1 | 23.5 | 14.8 | 8.0 | 2.9 | 11.0 | 0.67 |
| FG8 | 5.3 | 2.9 | 30.5 | 0.0 | 13.5 | 1.4 | 20.2 | 18.1 | 8.2 | 0.0 | 8.2 | 0.58 |
| FG9 | 4.6 | 3.2 | 27.4 | 0.0 | 12.9 | 1.5 | 20.6 | 19.6 | 6.7 | 2.5 | 9.2 | 0.61 |
| FG10 | 5.5 | 3.0 | 24.4 | 1.1 | 15.6 | 1.3 | 22.1 | 17.5 | 6.5 | 2.1 | 8.6 | 0.65 |
| FG11 | 5.2 | 3.4 | 32.4 | 0.0 | 8.0 | 1.5 | 17.1 | 20.4 | 7.8 | 4.2 | 12.0 | 0.53 |
| FG12 | 5.1 | 3.1 | 34.0 | 0.0 | 12.5 | 1.3 | 19.4 | 17.5 | 7.2 | 0.0 | 7.2 | 0.53 |
| FG13 | 4.8 | 2.6 | 31.0 | 0.0 | 11.5 | 1.1 | 21.2 | 19.0 | 6.1 | 2.6 | 8.7 | 0.57 |
| FG14 | 5.1 | 3.3 | 31.6 | 1.2 | 9.3 | 1.4 | 16.2 | 19.4 | 8.0 | 3.4 | 11.4 | 0.54 |
| FG15 | 5.1 | 2.6 | 27.8 | 0.0 | 13.4 | 1.1 | 22.5 | 16.9 | 7.0 | 2.6 | 9.5 | 0.62 |
| FG16 | 5.3 | 3.0 | 28.2 | 1.1 | 12.3 | 1.3 | 19.0 | 19.1 | 7.0 | 2.7 | 9.7 | 0.59 |
| FG17 | 5.2 | 2.9 | 32.1 | 1.2 | 10.7 | 1.2 | 17.0 | 19.5 | 6.7 | 2.6 | 9.3 | 0.54 |
| FG18 | 5.0 | 2.5 | 30.5 | 1.0 | 10.4 | 1.0 | 19.9 | 18.7 | 7.0 | 3.0 | 10.1 | 0.57 |
| FG19 | 5.0 | 3.2 | 29.0 | 0.9 | 10.9 | 1.3 | 21.8 | 18.0 | 6.2 | 2.7 | 8.9 | 0.59 |
| FG20 | 4.9 | 3.3 | 34.9 | 0.0 | 8.9 | 1.4 | 16.7 | 19.6 | 7.1 | 3.2 | 10.2 | 0.51 |
| FG21 | 5.3 | 2.7 | 27.5 | 1.1 | 13.4 | 1.1 | 19.9 | 17.0 | 8.1 | 2.9 | 11.0 | 0.62 |
| FG22 | 4.6 | 3.3 | 30.7 | 0.0 | 10.0 | 1.4 | 19.7 | 19.4 | 6.9 | 3.0 | 9.9 | 0.56 |
| BU18 | 6.0 | 3.3 | 15.6 | 1.9 | 48.7 | 0.4 | 1.6 | 0.0 | 21.5 | 0.0 | 21.5 | 0.82 |
| BU18 | 5.9 | 2.7 | 14.9 | 1.9 | 51.6 | 0.3 | 1.7 | 0.2 | 19.9 | 0.0 | 19.9 | 0.83 |
| FC1 | 6.0 | 2.5 | 13.6 | 1.7 | 53.2 | 0.4 | 1.5 | 0.2 | 20.1 | 0.0 | 20.1 | 0.85 |
| FC2 | 6.3 | 2.9 | 13.5 | 2.1 | 50.8 | 0.4 | 1.6 | 0.1 | 21.7 | 0.0 | 21.7 | 0.85 |
| FC3 | 6.0 | 2.7 | 14.5 | 2.0 | 53.3 | 0.4 | 1.5 | 0.0 | 19.0 | 0.0 | 19.0 | 0.84 |
| FC4 | 5.9 | 2.6 | 15.2 | 1.6 | 52.2 | 0.4 | 1.3 | 0.0 | 20.2 | 0.0 | 20.2 | 0.83 |
| FC5 | 6.2 | 2.7 | 14.7 | 1.9 | 53.6 | 0.4 | 1.4 | 0.2 | 18.3 | 0.0 | 18.3 | 0.83 |
| FC6 | 5.9 | 2.5 | 14.1 | 1.9 | 50.1 | 0.4 | 1.6 | 0.0 | 22.6 | 0.0 | 22.6 | 0.84 |
| FC7 | 5.9 | 2.4 | 14.2 | 1.9 | 52.4 | 0.4 | 1.5 | 0.0 | 20.8 | 0.0 | 20.8 | 0.84 |
| FC8 | 5.8 | 2.3 | 13.7 | 1.8 | 56.9 | 0.3 | 1.9 | 0.0 | 14.5 | 0.0 | 14.5 | 0.84 |
| FC9 | 6.5 | 3.2 | 13.9 | 2.0 | 51.3 | 0.5 | 1.6 | 0.1 | 20.1 | 0.0 | 20.1 | 0.84 |
| FC10 | 6.0 | 3.0 | 13.5 | 1.9 | 49.8 | 0.4 | 1.7 | 0.0 | 22.8 | 0.0 | 22.8 | 0.85 |
| FC11 | 6.1 | 3.0 | 14.8 | 2.0 | 49.4 | 0.5 | 1.7 | 0.2 | 21.8 | 0.0 | 21.8 | 0.83 |
| FC12 | 6.1 | 3.2 | 13.4 | 1.7 | 47.3 | 0.4 | 2.0 | 0.0 | 23.4 | 0.0 | 23.4 | 0.84 |
| FC13 | 6.8 | 3.1 | 13.2 | 2.4 | 47.5 | 0.4 | 2.1 | 0.0 | 23.7 | 0.0 | 23.7 | 0.85 |
| FC14 | 6.2 | 3.1 | 15.7 | 2.0 | 50.6 | 0.5 | 2.0 | 0.0 | 18.8 | 0.0 | 18.8 | 0.82 |
| FC15 | 6.0 | 2.3 | 12.0 | 1.9 | 54.5 | 0.4 | 2.2 | 0.0 | 19.7 | 0.0 | 19.7 | 0.86 |
| FC16 | 7.1 | 2.9 | 8.3 | 2.3 | 46.6 | 0.3 | 2.8 | 0.4 | 28.0 | 0.0 | 28.0 | 0.90 |
| FC17 | 6.0 | 2.4 | 13.8 | 1.9 | 55.6 | 0.4 | 1.9 | 0.0 | 17.1 | 0.0 | 17.1 | 0.84 |
| FC18 | 6.4 | 2.4 | 12.2 | 2.3 | 50.8 | 0.5 | 2.6 | 0.3 | 21.1 | 0.0 | 21.1 | 0.86 |

### Example 3

### Isolation and Expression of Genes Encoding Diacylglycerol Acyltransferase 1 (DGAT1)

Cloning of Arabidopsis thaliana AtDGAT1

A DNA fragment containing the full-length Arabidopsis thaliana protein coding region encoding diacylglycerol acyltransferase 1 gene (AtDGAT1; gene At2g19450) was amplified from stem cDNA with proof-reading polymerase PfuUltraII (Stratagene) and primers:—

AtDGAT1-F1
(SEQ ID NO: 86)
5′-TCGGGTACCGCTTTTCGAAATGGCGAT-3′
and
AtDGAT1-R1
(SEQ ID NO: 87)
5′-TTGGATATCGACGTCATGACATCGATCCTTTTC-3′

and inserted into a pBluescript SK (Stratagene) derivative, resulting in plasmid pXZP163. After confirming the nucleotide sequence of the coding region, the gene was cleaved out and subcloned into binary vector pWVec8-Fp1 (Singh et al., 2001), generating plasmid pXZP307, for expression in transgenic plants by the methods described in Example 1.
Cloning of Bernardia pulchella Gene Encoding DGAT1 (BpDGAT1) by Screening cDNA Library

A radioactive probe prepared from the full-length protein coding region of AtDGAT1, excised as a KpnI-EcoRV fragment from pXZP 163, was used as a probe to screen the B. pulchella cDNA library. The hybridization was performed at 55° C. overnight and the blots washed at 55° C. with 2×SSC/0.1% SDS twice for 10 minutes. Twelve positive plaques were selected for secondary screening, and one clone was confirmed as containing an insert with a sequence that hybridized strongly to the probe. After in vivo excision to remove the insert, the nucleotide sequence of the insert was determined (SEQ ID NO:44). The open reading frame encoding a protein started with the ATG start codon at nucleotides 75-77 and was terminated by the TGA stop codon at nucleotides 1725-1727. The deduced amino acid sequence of 550 amino acids is shown in SEQ ID NO:2. The gene was designated BpDGAT1 and the encoded protein exhibited 64% amino acid identity when compared to Arabidopsis AtDGAT1.

Scanning the BpDGAT1 protein sequence against the Prosite database (http://expasy.org/tools/scanprosite) identified three potential N-linked glycosylation sites, (residues 27-30, -NLSL-; 73-76, -NLSM-; 109-112, -NDSS-), 8 potential protein kinase C phosphorylation sites (residues 29-31, 112-114, 130-132, 140-142, 193-195, 196-198, 311-313, 335-337), 9 potential casein kinase II phosphorylation sites (residues 2-5, 38-41, 49-52, 66-69, 86-89, 140-143, 196-199, 282-285 and 431-434), one cAMP- and cGMP-dependent protein kinase phosphorylation site (residues 33-36, -RRWT-), one tyrosine kinase phosphorylation site (residues 416-423, -RFGDREFY-), two leucine zipper motifs (residues 246-267, -LypvsviLscesavLsgvtlmL-; 253-267, -LscesavLsgvtlmLfacivwL-) and two N-myristoylation sites (residues 20-25, 531-536).

### Expression of AtDGAT1 in Plants

AtDGAT1 in pXZP307 was introduced and expressed in transgenic plants of the Ven9 line. The percentages of epoxy fatty acids, namely 12,13-epoxy-oleic (18:1 Ep; vernolic acid); 12,13-epoxy linoleic (18:2 Ep) and the sum of the two epoxy fatty acids (Total Ep) as a percentage of total fatty acids in the seed oil of 18 transgenic lines were not significantly changed compared to parental line Ven9, as shown in Table 3. The vernolic acid level in seed oil from individual Ven9 plants grown at the same time and under the same conditions ranged from 5-9%.

### Expression of BpDGAT1

The full-length protein coding region of the BpDGAT1 cDNA was cloned into pENTR11 as a BamHI-XhoI fragment to generate the plasmid pXZP079E. The gene was then recombined into the yeast expression vector pYES-DEST52 and the plant expression vector pXZP391 by LR Clonase, resulting in pXZP237 and pXZP377, respectively. The DGAT function and substrate specificity of the gene expressed in transformed yeast cells is analyzed as described in Example 1.

The Arabidopsis lines Ven9 and BU18 were transformed with pXZP377 resulting in 21 and 23 transgenic lines, designated FB and FA, respectively. The vernolic acid levels (Ver) and ODP of transgenic seeds from these lines were shown in Table 4. A few lines expressing BpDGAT1 in Ven9 had increased levels of total epoxy fatty acids, while there was no obvious increase in the level in the transgenic BU18 seed. The epoxy fatty acid levels in the progeny of these lines are being studied.

TABLE 3
Seed oil composition of Arabidopsis line Ven9 and transgenic derivatives carrying the AtDGAT1 gene.

| Plant | 16:0 | 18:0 | 18:1 | 18:1n7 | 18:2 | 18:3 | 20:0 | 20:1 | 22:1 | Ver | 18:2E | Total Epoxy | ODP |
|---|---|---|---|---|---|---|---|---|---|---|---|---|---|
| Ven9 | 7.2 | 4.4 | 36.3 | 1.1 | 7.5 | 7.1 | 2.0 | 23.7 | 1.3 | 5.0 | 1.5 | 6.5 | 0.37 |
| CN2 | 6.6 | 3.8 | 31.0 | 0.7 | 14.2 | 11.0 | 1.8 | 23.2 | 1.2 | 3.4 | 0.6 | 4.0 | 0.48 |
| CN3 | 7.7 | 3.8 | 29.3 | 0.9 | 15.3 | 9.9 | 1.5 | 22.4 | 1.0 | 4.5 | 0.7 | 5.1 | 0.51 |
| CN4 | 6.9 | 4.0 | 37.4 | 0.8 | 6.9 | 7.6 | 1.7 | 25.1 | 1.0 | 4.9 | 1.3 | 6.2 | 0.36 |
| CN6 | 6.6 | 3.8 | 33.6 | 0.6 | 10.7 | 11.1 | 1.6 | 22.9 | 1.2 | 4.2 | 1.1 | 5.3 | 0.45 |
| CN7 | 8.2 | 3.8 | 36.6 | 0.6 | 11.6 | 7.1 | 1.5 | 20.7 | 0.9 | 4.7 | 1.3 | 6.0 | 0.40 |
| CN9 | 6.6 | 4.8 | 39.3 | 0.7 | 7.2 | 7.6 | 1.8 | 22.4 | 0.9 | 4.8 | 1.5 | 6.3 | 0.35 |
| CN10 | 6.4 | 3.2 | 30.1 | 0.7 | 13.5 | 13.7 | 1.5 | 20.7 | 1.2 | 4.9 | 1.0 | 5.9 | 0.52 |
| CN11 | 6.8 | 4.1 | 37.0 | 0.6 | 8.2 | 9.0 | 1.7 | 22.2 | 1.1 | 4.3 | 1.1 | 5.4 | 0.38 |
| CN12 | 6.9 | 4.3 | 39.7 | 0.6 | 5.9 | 6.6 | 1.8 | 23.5 | 1.1 | 5.1 | 1.4 | 6.5 | 0.32 |
| CN13 | 6.0 | 3.9 | 35.9 | 0.7 | 8.2 | 8.2 | 1.7 | 24.6 | 1.1 | 5.6 | 1.4 | 7.0 | 0.39 |
| CN14 | 7.3 | 4.0 | 31.9 | 0.8 | 9.3 | 9.3 | 1.7 | 22.4 | 1.0 | 7.1 | 1.6 | 8.8 | 0.46 |
| CN15 | 6.0 | 3.9 | 40.7 | 0.5 | 6.0 | 6.8 | 1.6 | 24.4 | 1.0 | 5.3 | 1.4 | 6.7 | 0.32 |
| CN16 | 6.2 | 4.0 | 38.6 | 0.6 | 6.4 | 6.6 | 1.7 | 25.5 | 1.1 | 5.5 | 1.5 | 7.0 | 0.34 |
| CN17 | 5.7 | 3.9 | 36.6 | 0.5 | 8.9 | 10.0 | 1.7 | 24.1 | 1.1 | 4.2 | 1.1 | 5.3 | 0.40 |
| CN18 | 6.8 | 4.3 | 36.6 | 0.6 | 7.1 | 7.0 | 2.0 | 24.5 | 1.1 | 5.4 | 1.5 | 6.9 | 0.36 |
| CN19 | 6.3 | 3.9 | 36.4 | 0.6 | 7.7 | 9.3 | 1.7 | 24.2 | 1.2 | 4.3 | 1.3 | 5.5 | 0.38 |
| CN20 | 6.4 | 3.2 | 34.9 | 1.0 | 11.6 | 7.6 | 1.4 | 23.3 | 1.0 | 5.5 | 0.8 | 6.3 | 0.42 |

TABLE 4
Seed oil composition of Arabidopsis Ven9 or BU18 lines and transgenic derivatives carrying the BpDGAT1 gene.

| | | | | | | | | | | | Total | |
|---|---|---|---|---|---|---|---|---|---|---|---|---|
| Plant | C16:0 | C18:0 | C18:1 | C18:1n7 | C18:2 | C20:0 | C18:3 | C20:1 | Ver | C18:2E | Epoxy | ODP |
| Ven9 | 5.0 | 3.4 | 32.2 | 1.1 | 8.2 | 1.2 | 17.9 | 18.8 | 7.0 | 3.4 | 10.4 | 0.53 |
| FB2 | 5.3 | 3.0 | 24.3 | 1.0 | 19.4 | 1.2 | 25.0 | 15.1 | 5.6 | 0.0 | 5.6 | 0.67 |
| FB3 | 5.4 | 3.0 | 29.7 | 0.0 | 16.8 | 1.1 | 24.8 | 14.0 | 5.3 | 0.0 | 5.3 | 0.61 |
| FB4 | 5.6 | 2.7 | 30.1 | 1.3 | 15.5 | 1.1 | 19.0 | 17.1 | 7.7 | 0.0 | 7.7 | 0.58 |
| FB5 | 5.1 | 2.5 | 34.8 | 0.0 | 13.2 | 0.0 | 23.9 | 14.0 | 6.6 | 0.0 | 6.6 | 0.56 |
| FB6 | 5.0 | 2.2 | 15.6 | 0.9 | 29.4 | 0.9 | 32.7 | 12.3 | 0.0 | 0.0 | 0.0 | 0.80 |
| FB7 | 5.6 | 2.7 | 24.7 | 1.1 | 17.8 | 1.0 | 28.2 | 13.9 | 4.9 | 0.0 | 4.9 | 0.67 |
| FB8 | 5.2 | 3.0 | 36.9 | 1.3 | 8.0 | 1.1 | 16.0 | 16.6 | 8.0 | 3.8 | 11.8 | 0.49 |
| FB9 | 5.1 | 2.4 | 23.8 | 0.0 | 16.6 | 1.1 | 28.9 | 15.4 | 6.8 | 0.0 | 6.8 | 0.69 |
| FB10 | 5.5 | 2.8 | 32.1 | 0.0 | 12.0 | 0.0 | 23.5 | 16.7 | 7.4 | 0.0 | 7.4 | 0.57 |
| FB11 | 5.6 | 3.2 | 24.2 | 1.1 | 15.5 | 1.4 | 26.9 | 17.0 | 5.2 | 0.0 | 5.2 | 0.66 |
| FB12 | 5.1 | 2.7 | 26.0 | 1.1 | 19.1 | 0.9 | 28.0 | 12.2 | 4.9 | 0.0 | 4.9 | 0.67 |
| FB13 | 5.3 | 3.0 | 18.5 | 0.8 | 21.1 | 1.3 | 32.3 | 13.8 | 2.5 | 0.0 | 2.5 | 0.75 |
| FB14 | 5.4 | 3.2 | 36.5 | 0.0 | 6.7 | 1.2 | 15.4 | 17.6 | 9.0 | 5.1 | 14.1 | 0.50 |
| FB16 | 5.6 | 3.3 | 34.2 | 1.4 | 10.3 | 1.1 | 16.7 | 15.6 | 8.1 | 3.6 | 11.7 | 0.53 |
| FB17 | 5.5 | 3.0 | 38.3 | 0.0 | 6.4 | 1.2 | 13.1 | 18.6 | 9.4 | 4.5 | 13.9 | 0.47 |
| FB18 | 4.8 | 2.9 | 30.8 | 1.0 | 12.1 | 1.1 | 20.4 | 17.1 | 6.6 | 2.4 | 9.0 | 0.57 |
| FB19 | 5.1 | 2.4 | 25.6 | 1.1 | 20.2 | 0.9 | 20.6 | 14.9 | 7.1 | 1.4 | 8.5 | 0.66 |
| FB20 | 5.2 | 2.4 | 28.9 | 1.1 | 16.0 | 0.9 | 23.1 | 16.2 | 6.1 | 0.0 | 6.1 | 0.61 |
| FB21 | 5.2 | 3.1 | 31.5 | 0.0 | 8.7 | 1.3 | 20.0 | 17.0 | 8.5 | 4.7 | 13.2 | 0.57 |
| FB22 | 5.0 | 3.3 | 28.7 | 0.9 | 10.6 | 1.2 | 24.1 | 16.8 | 5.8 | 2.8 | 8.6 | 0.60 |
| FB23 | 5.6 | 2.6 | 30.6 | 0.0 | 13.8 | 0.0 | 21.0 | 14.0 | 12.5 | 0.0 | 12.5 | 0.61 |
| FB24 | 5.1 | 2.8 | 26.1 | 0.9 | 11.3 | 1.3 | 21.1 | 18.9 | 8.5 | 4.0 | 12.6 | 0.63 |
| FB25 | 4.9 | 2.8 | 19.0 | 1.1 | 24.0 | 1.2 | 34.4 | 12.6 | 0.0 | 0.0 | 0.0 | 0.75 |
| BU18 | 6.0 | 3.3 | 15.6 | 1.9 | 48.7 | 0.4 | 1.6 | 0.0 | 21.5 | 0.0 | 21.5 | 0.82 |
| BU18 | 5.9 | 2.7 | 14.9 | 1.9 | 51.6 | 0.3 | 1.7 | 0.2 | 19.9 | 0.0 | 19.9 | 0.83 |
| FA1 | 6.0 | 2.2 | 14.4 | 1.5 | 56.4 | 0.0 | 0.0 | 0.0 | 19.5 | 0.0 | 19.5 | 0.84 |
| FA2 | 5.9 | 2.1 | 14.9 | 1.8 | 57.7 | 0.0 | 0.0 | 0.0 | 17.7 | 0.0 | 17.7 | 0.83 |
| FA3 | 6.1 | 2.6 | 16.1 | 1.9 | 56.3 | 0.0 | 1.4 | 0.0 | 15.7 | 0.0 | 15.7 | 0.82 |
| FA4 | 5.7 | 2.3 | 16.0 | 1.6 | 56.5 | 0.0 | 0.0 | 0.0 | 17.9 | 0.0 | 17.9 | 0.82 |
| FA5 | 6.2 | 2.5 | 14.9 | 1.8 | 57.8 | 0.0 | 0.0 | 0.0 | 16.8 | 0.0 | 16.8 | 0.83 |
| FA6 | 6.0 | 2.5 | 16.7 | 1.7 | 57.4 | 0.0 | 0.0 | 0.0 | 15.7 | 0.0 | 15.7 | 0.81 |
| FA7 | 5.7 | 2.1 | 15.6 | 1.9 | 57.7 | 0.3 | 1.4 | 0.0 | 15.3 | 0.0 | 15.3 | 0.83 |
| FA9 | 5.4 | 2.2 | 15.6 | 1.8 | 53.8 | 0.0 | 1.4 | 0.0 | 19.7 | 0.0 | 19.7 | 0.83 |
| FA10 | 6.1 | 2.5 | 15.7 | 1.9 | 58.9 | 0.0 | 1.3 | 0.0 | 13.6 | 0.0 | 13.6 | 0.82 |
| FA11 | 5.8 | 2.1 | 16.7 | 1.9 | 59.5 | 0.3 | 1.5 | 0.0 | 12.3 | 0.0 | 12.3 | 0.81 |
| FA12 | 5.9 | 2.5 | 16.0 | 1.9 | 54.1 | 0.0 | 0.0 | 0.0 | 19.6 | 0.0 | 19.6 | 0.82 |
| FA13 | 6.2 | 2.8 | 15.3 | 1.7 | 56.5 | 0.0 | 0.0 | 0.0 | 17.6 | 0.0 | 17.6 | 0.83 |
| FA14 | 6.1 | 2.8 | 16.1 | 1.7 | 57.4 | 0.0 | 0.0 | 0.0 | 15.8 | 0.0 | 15.8 | 0.82 |
| FA15 | 6.2 | 2.5 | 15.6 | 1.7 | 57.5 | 0.0 | 1.5 | 0.0 | 15.1 | 0.0 | 15.1 | 0.83 |
| FA17 | 6.1 | 3.0 | 16.0 | 1.7 | 54.6 | 0.0 | 0.0 | 0.0 | 18.6 | 0.0 | 18.6 | 0.82 |
| FA18 | 6.0 | 2.6 | 14.8 | 1.5 | 56.0 | 0.0 | 0.0 | 0.0 | 19.1 | 0.0 | 19.1 | 0.84 |
| FA19 | 5.6 | 2.3 | 19.1 | 1.6 | 57.9 | 0.0 | 0.0 | 0.0 | 13.4 | 0.0 | 13.4 | 0.79 |
| FA20 | 6.0 | 2.7 | 14.5 | 1.8 | 56.0 | 0.0 | 0.0 | 0.0 | 19.1 | 0.0 | 19.1 | 0.84 |
| FA21 | 6.4 | 2.7 | 15.7 | 1.6 | 55.5 | 0.0 | 0.0 | 0.0 | 18.0 | 0.0 | 18.0 | 0.82 |
| FA22 | 5.8 | 2.3 | 14.7 | 1.7 | 57.5 | 0.0 | 1.5 | 0.0 | 16.6 | 0.0 | 16.6 | 0.84 |
| FA23 | 6.0 | 2.7 | 14.5 | 1.6 | 56.4 | 0.0 | 0.0 | 0.0 | 18.7 | 0.0 | 18.7 | 0.84 |
| FA24 | 6.2 | 2.5 | 15.5 | 1.7 | 56.6 | 0.0 | 0.0 | 0.0 | 17.4 | 0.0 | 17.4 | 0.83 |
| FA25 | 5.7 | 2.6 | 16.1 | 1.5 | 55.6 | 0.0 | 1.3 | 0.0 | 17.2 | 0.0 | 17.2 | 0.82 |

### Example 4

### Isolation and Expression of a Gene Encoding B. pulchella Diacylglycerol Acyltransferase 3 (BpDGAT3)

DGAT3 is a diacylglycerol acyltransferase identified from peanut (Arachis hypogaea, Saha et al., 2006) and its gene recently cloned. In contrast to DGAT1 and DGAT2 which are ER membrane-associated proteins, DGAT3 was found to be a soluble enzyme without membrane spanning domains or signal sequences for translocation across membranes. Furthermore, in Arabidopsis, DGAT1 mRNA was expressed at high levels in many different tissues, including germinating seeds, young seedlings, roots, and leaves. However, the soluble DGAT3 protein in peanut was detected only in immature, developing seeds.

### Cloning of BpDGAT3

When the amino acid sequences obtained from the 12,180 nucleotide sequences of the EST collection (Example 2) were screened by BlastX, twelve partial length cDNA clones were identified that shared sequence homology with peanut (Arachis hypogaea) soluble diacylglycerol acyltransferase AhDGAT (Accession No. AY875644, Saha et al., 2006), considered to be a DGAT3. The 12 clones had identical sequences in overlapping regions. The cDNA insert from one of the clones, Bp200867, was used as a probe to screen the cDNA library under high stringency conditions. Eight clones were identified, and one of them was sequenced and shown to contain a full-length cDNA. The open reading frame encoding the DGAT3 protein started with the ATG start codon at nucleotides 73-75 and was terminated by the TAG stop codon at nucleotides 1060-1062 (SEQ ID NO:45). The resultant amino acid sequence of this clone is shown in SEQ ID NO:3. The sequence of 329 amino acids showed 30% identity and 41% similarity to the peanut soluble DGAT3, AhDGAT, and 33% identity and 44% similarity to an Arabidopsis DGAT-like sequence (AAD49767). This clone therefore contained a cDNA for a gene designated as BpDGAT3.

BpDGAT3 has a serine rich region (-SESSTTSSSSSSES-). Scanning the BpDGAT3 protein sequence against the Prosite database (http://expasy.org/tools/scanprosite) identified five potential protein kinase C phosphorylation sites (residues 7-9, 52-54, 117-119, 222-224, 237-239), three casein kinase II phosphorylation sites (residues 85-88, 138-141, 140-143), five N-myristoylation sites (residues 41-46, 46-51, 230-235, 302-307, 323-328) and one leucine zipper pattern (residues 86-107, -LqdasraLmqqleeLkakekeL-).

### Expression of BpDGAT3

The full-length BpDGAT3 cDNA was cloned into pENTR11 as a BamHI-Bsp120I DNA fragment, after blunt ending, to generate plasmid pXZP093E. The gene was then recombined by LR Clonase reactions into pYES-DEST52 and pXZP391, resulting in pXZP246 and pXZP366, respectively. The DGAT function and substrate specificity of the gene expressed in transformed yeast cells is analyzed as described in Example 1.

When pXZP366 was used to transform Arabidopsis, transgenic lines designated GV and GW were generated in plants Ven9 and BU18, respectively, as described in Example 1.

### Example 5

### Isolation and Expression of a Gene Encoding B. pulchella Phospholipase A2 (BpPLA2)

The initial step of lipid hydrolysis is catalysed by phospholipases. These enzymes are grouped into four major classes, phospholipase A₁ and A₂, phospholipase C (PLC) and phospholipase D (PLD). The phospholipase A₂ (PLA₂) family of proteins include enzymes defined by their ability to specifically catalyse the hydrolysis of the middle (sn-2) ester bond of substrate phospholipids (Schaloske et al., 2006). The hydrolysis products of this reaction are free fatty acid and lysophospholipid. The free fatty acids released by PLA₂ can be assembled into TAG via the Kennedy pathway. The other product of PLA₂ enzyme catalysis, lysophospholipid, functions in cell signaling, phospholipid remodeling and membrane perturbation. More importantly, the unusual fatty acid, for example ricinoleic acid or vernolic acid, synthesized at the sn-2 position of phospholipid PC can be released by PLA₂, and subsequently incorporated into TAG in seed oil. PLA₂ enzymes have currently been classified into 15 Groups and many subgroups and include five distinct types of enzymes, namely the secreted PLA₂s (sPLA₂), the cytosolic PLA₂s (cPLA₂), the Ca²⁺ independent PLA₂s (iPLA₂), the platelet-activating factor acetylhydrolases (PAF-AH), and the lysosomal PLA₂s.

### Cloning of BpPLA2

When the amino acid sequences obtained from the EST collection were screened by BlastX, three cDNA clones (Bp205595, Bp210054 and Bp210422) were identified that encoded proteins that were homologous to the protein sequence for an Arabidopsis phospholipase A₂ (At2g06925, Accession No. NP_{—}565337), which is one of the secretory PLA₂. The sequences from these three clones were identical in the overlapping regions, and all contained a full-length protein coding sequence. SEQ ID NOs:46 and 4 are the full-length nucleotide sequence and deduced amino acid sequence, respectively, from the longest cDNA clone Bp205595. The open reading frame encoding the BpPLA2 protein started with the ATG start codon at nucleotides 71-73 and was terminated by the TAA stop codon at nucleotides 533-535, and encoded a protein of 154 amino acids (SEQ ID NO:4).

### Expression of BpPLA2

The protein coding region of the BpPLA2 cDNA clone Bp205595 was subcloned as an EcoRI-XhoI fragment into pENTR11, resulting in entry plasmid pXZP082E. The gene was recombined from this plasmid into yeast expression vector pYES-DEST52 and plant expression vector pXZP391, resulting in pXZP239 and pXZP380, respectively. The PLA2 function and substrate specificity of the gene expressed in transformed yeast cells is analyzed as described in Example 1.

Transformation of pXZP380 in plants Ven9 and BU18 generated 22 FH and 4 FD transgenic lines, respectively. GC analysis of fatty acid composition of seed oil of T2 seeds is shown in Table 5.

TABLE 5
Seed oil composition of Arabidopsis Ven9 or BU18 lines and transgenic derivatives carrying the BpPLA2 gene.

| Plant | C16:0 | C18:0 | C18:1 | C18:1n7 | C18:2 | C20:0 | C18:3 | C20:1 | Ver | C18:2E | Total Epoxy | ODP |
|---|---|---|---|---|---|---|---|---|---|---|---|---|
| Ven9 | 5.7 | 3.4 | 30.3 | 0.0 | 10.0 | 0.0 | 23.8 | 17.1 | 6.0 | 2.7 | 8.7 | 0.58 |
| FH1 | 4.8 | 3.1 | 32.4 | 0.0 | 11.4 | 1.2 | 18.4 | 17.6 | 7.4 | 2.8 | 10.2 | 0.55 |
| FH2 | 5.0 | 2.8 | 27.7 | 0.9 | 13.1 | 1.1 | 21.0 | 17.9 | 6.8 | 2.6 | 9.4 | 0.61 |
| FH3 | 5.3 | 3.0 | 30.7 | 0.0 | 10.1 | 1.3 | 19.6 | 18.2 | 7.9 | 3.9 | 11.8 | 0.58 |
| FH4 | 5.5 | 3.0 | 25.9 | 1.0 | 12.0 | 1.3 | 20.1 | 17.8 | 8.9 | 3.6 | 12.5 | 0.63 |
| FH5 | 5.1 | 3.3 | 29.9 | 0.0 | 11.2 | 1.3 | 19.0 | 17.2 | 8.5 | 3.6 | 12.1 | 0.59 |
| FH6 | 5.4 | 2.6 | 28.6 | 0.0 | 11.7 | 1.1 | 18.9 | 17.6 | 10.4 | 3.8 | 14.2 | 0.61 |
| FH7 | 5.2 | 2.8 | 34.9 | 0.0 | 10.7 | 1.2 | 17.3 | 18.8 | 9.1 | 0.0 | 9.1 | 0.52 |
| FH8 | 5.3 | 2.8 | 28.7 | 1.1 | 12.5 | 1.2 | 19.4 | 18.3 | 7.9 | 2.9 | 10.9 | 0.60 |
| FH9 | 4.7 | 2.8 | 25.2 | 0.0 | 18.0 | 1.3 | 22.8 | 17.6 | 7.7 | 0.0 | 7.7 | 0.66 |
| FH10 | 4.7 | 3.0 | 33.3 | 0.0 | 8.3 | 1.2 | 16.2 | 19.3 | 9.8 | 4.2 | 14.0 | 0.54 |
| FH11 | 0.0 | 0.0 | 65.0 | 0.0 | 0.0 | 0.0 | 0.0 | 35.0 | 0.0 | 0.0 | 0.0 | 0.00 |
| FH12 | 5.3 | 2.9 | 28.1 | 0.0 | 10.5 | 1.2 | 19.8 | 16.4 | 10.7 | 5.1 | 15.8 | 0.62 |
| FH13 | 5.0 | 2.6 | 22.4 | 1.1 | 18.2 | 1.2 | 22.5 | 18.1 | 6.1 | 1.8 | 7.9 | 0.68 |
| FH14 | 5.1 | 2.8 | 23.2 | 1.0 | 20.1 | 1.1 | 27.1 | 16.0 | 3.6 | 0.0 | 3.6 | 0.69 |
| FH15 | 5.1 | 2.5 | 36.7 | 0.0 | 9.3 | 0.9 | 15.9 | 17.6 | 8.5 | 3.5 | 11.9 | 0.50 |
| FH16 | 6.3 | 0.0 | 32.3 | 0.0 | 16.6 | 0.0 | 24.8 | 20.1 | 0.0 | 0.0 | 0.0 | 0.56 |
| FH18 | 5.6 | 2.4 | 29.8 | 1.3 | 9.9 | 0.0 | 17.1 | 16.8 | 12.3 | 4.7 | 17.0 | 0.60 |
| FH19 | 5.3 | 2.8 | 33.6 | 1.1 | 8.4 | 1.1 | 17.0 | 17.3 | 9.1 | 4.2 | 13.3 | 0.54 |
| FH20 | 4.8 | 3.4 | 32.7 | 0.0 | 9.6 | 1.5 | 18.3 | 17.3 | 8.6 | 3.8 | 12.4 | 0.55 |
| FH21 | 4.8 | 3.6 | 29.0 | 1.0 | 8.9 | 1.6 | 17.4 | 20.0 | 8.5 | 4.1 | 12.6 | 0.57 |
| FH22 | 4.8 | 2.7 | 32.4 | 0.0 | 10.6 | 1.1 | 18.6 | 18.9 | 7.7 | 3.3 | 11.0 | 0.55 |
| FH23 | 5.0 | 2.5 | 29.7 | 1.0 | 12.6 | 0.9 | 20.7 | 16.8 | 7.4 | 2.6 | 10.0 | 0.59 |
| FH24 | 5.0 | 2.5 | 28.2 | 0.9 | 11.8 | 1.0 | 19.2 | 17.2 | 9.5 | 3.7 | 13.2 | 0.61 |
| BU18 | 7.0 | 3.7 | 15.5 | 2.3 | 51.3 | 0.5 | 2.0 | 0.0 | 17.5 | 0.0 | 17.5 | 0.82 |
| BU18 | 7.2 | 3.8 | 14.5 | 2.2 | 51.9 | 0.5 | 2.1 | 0.2 | 17.5 | 0.0 | 17.5 | 0.83 |
| BU18 | 6.9 | 3.4 | 13.9 | 2.1 | 51.1 | 0.4 | 2.0 | 0.0 | 19.9 | 0.0 | 19.9 | 0.84 |
| FD1 | 6.3 | 2.5 | 14.2 | 2.0 | 56.2 | 0.0 | 0.0 | 0.0 | 18.8 | 0.0 | 18.8 | 0.84 |
| FD2 | 5.6 | 2.5 | 15.5 | 1.6 | 53.9 | 0.0 | 0.0 | 0.0 | 20.9 | 0.0 | 20.9 | 0.83 |
| FD3 | 5.7 | 2.6 | 15.8 | 1.9 | 52.6 | 0.0 | 0.0 | 0.0 | 21.5 | 0.0 | 21.5 | 0.82 |
| FD4 | 5.6 | 2.5 | 15.3 | 1.6 | 55.3 | 0.0 | 0.0 | 0.0 | 19.6 | 0.0 | 19.6 | 0.83 |

### Example 6

### Isolation and Expression of a Gene Encoding B. pulchella Phosphatidylcholine Diacylglycerol Acyltransferase (BpPDAT)

Cloning of A. thaliana AtPDAT by PCR

The protein coding region of the A. thaliana gene encoding diacylglycerol acyltransferase, AtPDAT (gene At5g13640), was amplified from A. thaliana (ecotype Columbia) leaf cDNA with proof-reading polymerase PfuUltraII (Stratagene) and oligonucleotide primers

AtPDAT-F1
(SEQ ID NO: 88)
5′- TTAGGTACCAGTGACAGATATGCCCCTT-3′
and
AtPDAT-R1
(SEQ ID NO: 89)
5′- ATGGAGCTCACAGCTTCAGGTCAATAC-3′,

and cloned as a KpnI-SacI fragment into a pBluescript SK derivative, resulting in plasmid pXZP161. After confirming the sequence, the gene was cloned into plant expression vectors pWVec8-Fp1 (Singh et al., 2001) and pGNAP (Lee et al., 1998), resulting in plasmid pXZP306 and pXZP308, carrying Hph and NptII selectable marker genes, respectively.
Gene Cloning of Euphorbia lagascae ElPDAT by cDNA Library Screening

A cDNA library in the vector λ ZAP II (Stratagene) was prepared from mRNA obtained from E. lagascae developing embryos in a similar fashion as described for B. pulchella in Example 1. The KpnI-SacI fragment from pXZP161 containing the entire protein coding sequence of AtPDAT1 was used as probe to screen the library by hybridization at 60° C., and the membranes were washed in 1×SSC/0.1% SDS at 55° C. Three hybridizing plaques were identified and sequenced after in vivo excision of the inserts. The sequences of all three cDNA clones were partial length and showed homology to AtPDAT. The longest of the clones, designated 1510, shared 37% identity and 42% similarity to the amino acid sequence of AtPDAT. The XbaI-HincII cDNA fragment from clone 1510 was used as a probe to re-screen the E. lagascae cDNA library at 60° C. The membranes were washed twice at 60° C. in 2×SSC/0.1% SDS each for 10 min, and in 02.×SSC/0.1% SDS for 10 min. Twenty-six plaques were picked for secondary screening using the same hybridisation and washing conditions. Nine positive plaques from the secondary screening were analyzed using ElPDAT-specific PCR. Five of them were processed by in vivo excision, and the cDNA sequence of the clone with the longest insert obtained, this is shown as SEQ ID NO:47. The open reading frame encoding the E1PDAT protein started with the ATG start codon at nucleotides 266-268 and was terminated by the TGA stop codon at nucleotides 1799-1801. The deduced amino acid sequence is shown in SEQ ID NO:S. The encoded protein of 511 amino acids was 150 amino acid residues shorter than AtPDAT, and had 50.3% amino acid identity and 60.8% amino acid similarity to AtPDAT in the overlapping region.

Gene Cloning of B. pulchella BpPDAT by cDNA Library Screening The XbaI-HincII fragment from E. lagascae PDAT cDNA clone 1510 containing the partial protein coding sequence was also used as a probe to screen the B. pulchella cDNA library at a hybridization temperature of 55° C. The membranes were washed 3 times at 60° C. in 2×SSC/0.1% SDS each for 10 min, and then once in 1×SSC/0.1% SDS for 10 min Twenty-six plaques were picked for secondary screening using the same conditions. Ten positive hybridizing plaques were selected from the secondary screening. Two of them were processed by in vivo excision, and the cDNA sequence of the clone Bp101529 with the longest insert determined. The nucleotide sequence is shown in SEQ ID NO:48. The open reading frame encoding the BpPDAT protein started with the ATG start codon at nucleotides 208-210 and was terminated by the TGA stop codon at nucleotides 2254-2256. The deduced amino acid sequence of 682 amino acids shared 76.3% amino acid identity and 82.9% similarity to AtPDAT, and is shown in SEQ ID NO:6.

### Expression of AtPDAT

The plasmid pXZP306 was used to transform Ven9 plants. Expression of the AtPDAT gene the transformed plants increased ODP levels, but reduced the vernolic acid levels (Table 6). Expression of the AtPDAT gene in Ven9 plants after transformation with plasmid pXZP308 using the nptII gene as selectable marker rather than hph plants led to similar results. It is possibly that the AtPDAT has preference for oleoyl-PC or linoleoyl-PC relative to vernoyl-PC as one substrate for incorporation of the acyl group into TAG, thus reduced the available epoxygenase substrate (vernoloyl-PC). It also indicated that merely increasing PDAT enzyme activity per se would not increase the level of the unusual fatty acid in the seed oil. Indeed, the data suggested that decreasing the endogenous activity of PDAT in the oilseed plant might contribute to increasing the level of the MFA in TAG of seed oil.

### Expression of ElPDAT

An EcoRI-XhoI fragment from E. lagascae PDAT cDNA clone 1510 was inserted into pENTR11, resulted in entry vector pXZP084E. The gene was then inserted into yeast expression vector pYES-DEST52 and plant expression vector pXZP391 by Clonase LR recombinase reactions, generating plasmids pXZP241 and pXZP382, respectively. pXZP382 was used to transform Ven9 plants and BU18 plants, generating 51 GM and 20 GP transgenic lines, respectively. GC analysis of fatty acid composition of seed oil of T2 seeds is shown in Table 7.

### Expression of BpPDAT

The XbaI-SphI fragment from Bp101529 containing the BpPDAT gene was inserted into pENTR11, resulted in entry vector pXZP081E. The gene was then cloned into yeast expression vector pYES-DEST52 and plant expression vector pXZP391 by Clonase LR recombinase reactions, generating plasmids pXZP240 and pXZP379, respectively. pXZP379 was used to transform Ven9 and BU18 plants, generating 35 GL and 45 GO transgenic lines, respectively. GC analysis of fatty acid composition of seed oil of T2 seeds is shown in Table 8.

TABLE 6
Seed oil composition of Arabidopsis Ven9 or BU18 lines and transgenic derivatives carrying the AtPDAT gene.

| | | | | | | | | | | | Total | |
|---|---|---|---|---|---|---|---|---|---|---|---|---|
| Plant | C16:0 | C18:0 | C18:1 | C18:1n7 | C18:2 | C20:0 | C18:3 | C20:1 | Ver | C18:2E | Epoxy | ODP |
| Ven9 | 7.5 | 4.3 | 34.8 | 2.2 | 6.7 | 1.9 | 7.4 | 23.0 | 5.6 | 1.8 | 7.5 | 0.38 |
| CK1 | 10.6 | 2.7 | 23.2 | 1.3 | 27.7 | 1.1 | 12.5 | 14.2 | 2.9 | 0.6 | 3.5 | 0.65 |
| CK2 | 8.3 | 2.8 | 29.8 | 0.5 | 20.5 | 1.3 | 11.6 | 18.1 | 2.8 | 0.5 | 3.3 | 0.54 |
| CK3 | 7.3 | 4.2 | 34.0 | 0.5 | 11.3 | 1.9 | 10.7 | 20.8 | 4.2 | 1.4 | 5.6 | 0.45 |
| CK4 | 6.1 | 3.3 | 42.1 | 0.5 | 7.8 | 1.4 | 7.8 | 21.8 | 4.6 | 1.4 | 6.0 | 0.34 |
| CK5 | 6.5 | 3.1 | 29.4 | 0.5 | 15.6 | 1.5 | 14.1 | 21.3 | 3.2 | 0.7 | 3.9 | 0.53 |
| CK6 | 6.0 | 3.4 | 37.6 | 0.5 | 8.9 | 1.5 | 9.8 | 23.1 | 3.9 | 1.2 | 5.1 | 0.39 |
| CK7 | 6.4 | 4.2 | 36.1 | 0.6 | 10.4 | 1.8 | 10.6 | 20.9 | 4.0 | 1.4 | 5.4 | 0.42 |
| CK8 | 6.5 | 4.1 | 29.3 | 1.1 | 12.8 | 2.0 | 14.2 | 21.9 | 3.0 | 0.9 | 3.9 | 0.51 |
| CK9 | 5.9 | 3.8 | 40.0 | 0.4 | 10.2 | 1.6 | 9.4 | 21.0 | 3.1 | 1.0 | 4.1 | 0.37 |
| CK10 | 6.6 | 3.4 | 30.6 | 0.5 | 16.2 | 1.5 | 15.6 | 19.7 | 1.9 | 0.4 | 2.3 | 0.53 |
| CK11 | 7.1 | 4.1 | 30.5 | 0.5 | 12.0 | 1.9 | 13.9 | 21.0 | 3.8 | 1.2 | 5.1 | 0.50 |
| CK12 | 7.0 | 3.4 | 26.3 | 1.5 | 16.0 | 1.7 | 14.8 | 20.7 | 3.2 | 0.8 | 3.9 | 0.57 |
| CK14 | 7.6 | 4.2 | 36.9 | 0.4 | 8.6 | 1.9 | 7.2 | 22.8 | 5.3 | 2.0 | 7.3 | 0.38 |
| CK18 | 6.3 | 3.5 | 35.2 | 0.5 | 9.5 | 1.6 | 11.6 | 23.2 | 3.2 | 1.0 | 4.2 | 0.42 |
| CK19 | 6.0 | 3.4 | 40.3 | 0.5 | 7.7 | 1.5 | 7.9 | 21.5 | 5.7 | 1.5 | 7.2 | 0.36 |
| CK22 | 6.6 | 3.1 | 25.1 | 1.5 | 19.7 | 1.6 | 14.5 | 20.4 | 2.3 | 0.3 | 2.6 | 0.59 |
| CK23 | 7.5 | 4.3 | 37.5 | 0.4 | 8.5 | 1.8 | 8.3 | 21.4 | 4.4 | 1.7 | 6.1 | 0.38 |

TABLE 7
Seed oil composition of Arabidopsis Ven9 or BU18 lines and transgenic derivatives carrying the E1PDAT gene.

| | | | | | | | | | | | Total | |
|---|---|---|---|---|---|---|---|---|---|---|---|---|
| Plant | C16:0 | C18:0 | C18:1 | C18:1n7 | C18:2 | C20:0 | C18:3 | C20:1 | Ver | C18:2E | Epoxy | ODP |
| Ven9 | 5.5 | 3.2 | 30.5 | 0.0 | 9.6 | 0.0 | 23.0 | 18.4 | 5.9 | 2.7 | 8.6 | 0.57 |
| Ven9 | 5.4 | 3.3 | 30.2 | 0.0 | 9.4 | 0.0 | 23.2 | 18.6 | 5.9 | 2.8 | 8.7 | 0.58 |
| Ven9 | 5.7 | 3.4 | 30.3 | 0.0 | 10.0 | 0.0 | 23.8 | 17.1 | 6.0 | 2.7 | 8.7 | 0.58 |
| GM1 | 5.0 | 3.0 | 27.5 | 0.0 | 11.7 | 0.0 | 23.9 | 19.3 | 6.1 | 2.1 | 8.3 | 0.61 |
| GM2 | 5.0 | 3.2 | 27.0 | 0.0 | 11.8 | 0.0 | 26.3 | 18.7 | 5.0 | 1.7 | 6.8 | 0.62 |
| GM3 | 5.0 | 2.8 | 28.0 | 0.0 | 11.2 | 0.0 | 23.1 | 20.2 | 5.9 | 2.2 | 8.1 | 0.60 |
| GM4 | 5.0 | 3.1 | 33.6 | 0.0 | 8.9 | 0.0 | 22.0 | 18.4 | 5.4 | 2.4 | 7.8 | 0.54 |
| GM5 | 4.8 | 3.1 | 30.4 | 0.0 | 11.1 | 0.0 | 24.1 | 18.7 | 4.6 | 1.8 | 6.5 | 0.58 |
| GM6 | 4.8 | 2.7 | 27.5 | 0.0 | 11.7 | 0.0 | 26.3 | 18.7 | 5.1 | 1.9 | 7.0 | 0.62 |
| GM8 | 5.9 | 3.7 | 35.8 | 0.0 | 0.1 | 0.0 | 23.5 | 20.5 | 6.3 | 2.6 | 8.8 | 0.48 |
| GM9 | 4.8 | 2.6 | 19.8 | 0.0 | 17.8 | 0.0 | 32.7 | 16.3 | 3.1 | 0.9 | 4.0 | 0.73 |
| GM10 | 5.0 | 2.7 | 31.1 | 0.0 | 11.7 | 0.0 | 23.7 | 17.8 | 4.9 | 1.8 | 6.7 | 0.58 |
| GM11 | 5.2 | 3.2 | 29.4 | 0.0 | 10.1 | 0.0 | 23.1 | 19.4 | 5.9 | 2.5 | 8.4 | 0.59 |
| GM12 | 5.7 | 2.6 | 34.9 | 0.0 | 10.1 | 0.0 | 17.4 | 20.3 | 5.6 | 2.0 | 7.7 | 0.50 |
| GM13 | 4.8 | 2.9 | 25.6 | 0.0 | 11.8 | 0.0 | 24.7 | 20.3 | 6.1 | 2.2 | 8.3 | 0.64 |
| GM14 | 5.4 | 3.8 | 28.2 | 0.0 | 9.6 | 0.0 | 22.4 | 21.1 | 5.6 | 2.2 | 7.8 | 0.59 |
| GM15 | 5.1 | 3.2 | 30.6 | 0.0 | 9.1 | 0.0 | 21.6 | 21.1 | 5.6 | 2.4 | 8.1 | 0.56 |
| GM16 | 5.4 | 3.1 | 25.2 | 0.0 | 12.5 | 0.0 | 25.1 | 20.1 | 5.1 | 2.0 | 7.0 | 0.64 |
| GM17 | 5.4 | 3.0 | 23.4 | 0.0 | 12.0 | 0.0 | 24.7 | 20.0 | 7.1 | 2.6 | 9.7 | 0.66 |
| GM18 | 5.6 | 2.9 | 24.4 | 0.0 | 14.8 | 0.0 | 24.3 | 19.1 | 5.7 | 1.6 | 7.3 | 0.66 |
| GM19 | 5.6 | 3.8 | 30.8 | 0.0 | 7.6 | 0.0 | 19.5 | 21.0 | 7.0 | 3.3 | 10.3 | 0.55 |
| GM20 | 5.1 | 3.1 | 25.7 | 0.0 | 11.2 | 0.0 | 25.9 | 20.1 | 5.4 | 2.2 | 7.6 | 0.63 |
| GM21 | 6.0 | 3.5 | 26.7 | 0.0 | 11.8 | 0.0 | 24.5 | 18.8 | 6.8 | 2.0 | 8.8 | 0.63 |
| GM22 | 4.6 | 2.9 | 17.0 | 0.0 | 19.1 | 0.0 | 32.8 | 17.7 | 3.1 | 0.8 | 3.9 | 0.77 |
| GM23 | 5.2 | 3.1 | 28.4 | 0.0 | 7.6 | 0.0 | 22.3 | 22.3 | 6.3 | 3.2 | 9.5 | 0.58 |
| GM24 | 5.0 | 2.7 | 28.4 | 0.0 | 11.1 | 0.0 | 24.6 | 18.7 | 5.7 | 2.4 | 8.0 | 0.61 |
| GM25 | 5.1 | 2.8 | 22.3 | 0.0 | 14.7 | 0.0 | 29.6 | 18.3 | 4.1 | 1.5 | 5.5 | 0.69 |
| GM26 | 5.2 | 3.3 | 21.7 | 0.0 | 13.4 | 0.0 | 25.8 | 20.7 | 6.0 | 2.0 | 8.0 | 0.68 |
| GM27 | 5.0 | 3.0 | 29.0 | 0.0 | 10.8 | 0.0 | 23.3 | 19.9 | 5.4 | 2.1 | 7.6 | 0.59 |
| GM28 | 5.7 | 3.4 | 27.3 | 0.0 | 10.1 | 0.0 | 23.7 | 19.2 | 6.2 | 2.9 | 9.1 | 0.61 |
| GM29 | 5.4 | 3.1 | 24.7 | 0.0 | 11.1 | 0.0 | 26.2 | 20.9 | 4.8 | 2.1 | 6.9 | 0.64 |
| GM30 | 5.1 | 2.7 | 20.8 | 0.0 | 15.1 | 0.0 | 27.8 | 18.8 | 5.9 | 1.9 | 7.7 | 0.71 |
| GM31 | 5.4 | 3.2 | 23.9 | 0.0 | 12.2 | 0.0 | 25.4 | 20.7 | 5.5 | 2.0 | 7.5 | 0.65 |
| GM32 | 5.3 | 3.1 | 18.2 | 0.0 | 17.2 | 0.0 | 29.2 | 18.5 | 5.2 | 1.5 | 6.7 | 0.74 |
| GM33 | 5.3 | 3.1 | 17.8 | 0.0 | 17.8 | 0.0 | 33.2 | 17.2 | 2.7 | 1.0 | 3.7 | 0.75 |
| GM34 | 5.7 | 3.2 | 23.0 | 0.0 | 14.2 | 0.0 | 26.4 | 17.4 | 6.8 | 2.1 | 8.9 | 0.68 |
| GM35 | 5.4 | 2.9 | 22.5 | 0.0 | 14.4 | 0.0 | 28.0 | 19.5 | 4.0 | 1.4 | 5.4 | 0.68 |
| GM36 | 5.4 | 3.4 | 25.1 | 0.0 | 10.7 | 0.0 | 25.4 | 20.4 | 5.7 | 2.4 | 8.1 | 0.64 |
| GM37 | 5.8 | 3.3 | 25.0 | 0.0 | 11.7 | 0.0 | 24.7 | 19.8 | 6.0 | 2.3 | 8.3 | 0.64 |
| GM38 | 4.9 | 2.8 | 15.5 | 0.0 | 20.3 | 0.0 | 34.4 | 17.7 | 2.2 | 0.0 | 2.2 | 0.79 |
| GM39 | 5.6 | 3.2 | 19.8 | 0.0 | 15.7 | 0.0 | 28.5 | 18.9 | 5.1 | 1.5 | 6.6 | 0.72 |
| GM40 | 5.1 | 3.2 | 30.3 | 0.0 | 8.7 | 0.0 | 20.7 | 21.7 | 6.1 | 2.7 | 8.8 | 0.56 |
| GM41 | 4.9 | 3.1 | 26.5 | 0.0 | 11.7 | 0.0 | 25.4 | 18.5 | 6.3 | 2.4 | 8.7 | 0.63 |
| GM42 | 5.5 | 3.5 | 28.7 | 0.0 | 8.0 | 0.0 | 22.1 | 21.8 | 5.9 | 2.9 | 8.8 | 0.58 |
| GM43 | 5.4 | 3.1 | 22.8 | 0.0 | 13.1 | 0.0 | 25.3 | 20.9 | 5.7 | 1.9 | 7.6 | 0.67 |
| GM44 | 5.5 | 2.9 | 30.6 | 0.0 | 8.8 | 0.0 | 20.8 | 20.8 | 6.2 | 2.6 | 8.8 | 0.56 |
| GM45 | 5.6 | 3.2 | 25.2 | 0.0 | 11.4 | 0.0 | 24.7 | 19.9 | 6.0 | 2.4 | 8.4 | 0.64 |
| GM46 | 5.4 | 2.5 | 17.7 | 0.0 | 19.7 | 0.0 | 30.2 | 17.0 | 4.3 | 1.1 | 5.4 | 0.76 |
| GM47 | 5.5 | 3.0 | 18.9 | 0.0 | 16.1 | 0.0 | 28.3 | 19.2 | 5.3 | 1.6 | 6.9 | 0.73 |
| GM48 | 5.4 | 2.8 | 20.4 | 0.0 | 14.2 | 0.0 | 27.8 | 19.9 | 5.6 | 1.9 | 7.5 | 0.71 |
| GM49 | 5.8 | 3.0 | 21.2 | 0.0 | 13.9 | 0.0 | 25.0 | 20.0 | 6.9 | 2.3 | 9.2 | 0.69 |
| GM50 | 5.9 | 3.4 | 29.8 | 0.0 | 8.2 | 0.0 | 20.7 | 21.6 | 6.1 | 2.7 | 8.8 | 0.56 |
| GM51 | 5.4 | 3.3 | 28.3 | 0.0 | 10.2 | 0.0 | 22.5 | 21.8 | 5.2 | 2.0 | 7.2 | 0.58 |
| BU18-1 | 6.4 | 3.0 | 16.5 | 2.1 | 55.7 | 0.3 | 1.6 | 0.2 | 13.8 | 0.0 | 13.8 | 0.81 |
| BU18-2 | 6.5 | 3.4 | 17.2 | 1.8 | 54.8 | 0.4 | 1.5 | 0.2 | 13.8 | 0.0 | 13.8 | 0.80 |
| BU18-3 | 7.3 | 3.4 | 17.1 | 1.7 | 53.7 | 0.4 | 1.5 | 0.0 | 13.9 | 0.0 | 13.9 | 0.80 |
| GP1 | 6.7 | 3.4 | 17.1 | 1.9 | 49.7 | 0.0 | 2.7 | 0.0 | 18.4 | 0.0 | 18.4 | 0.81 |
| GP2 | 6.8 | 3.5 | 17.0 | 1.9 | 53.5 | 0.0 | 2.9 | 0.3 | 13.8 | 0.0 | 13.8 | 0.81 |
| GP3 | 7.0 | 3.4 | 17.2 | 1.7 | 53.8 | 0.0 | 2.6 | 0.0 | 14.1 | 0.0 | 14.1 | 0.80 |
| GP4 | 6.7 | 3.5 | 18.1 | 1.6 | 56.5 | 0.4 | 1.8 | 0.0 | 11.1 | 0.0 | 11.1 | 0.79 |
| GP5 | 7.2 | 3.9 | 17.4 | 1.9 | 56.3 | 0.4 | 2.0 | 0.3 | 10.5 | 0.0 | 10.5 | 0.80 |
| GP6 | 7.0 | 4.0 | 15.4 | 2.2 | 55.0 | 0.5 | 1.9 | 0.0 | 13.7 | 0.0 | 13.7 | 0.82 |
| GP7 | 6.6 | 3.5 | 22.1 | 1.8 | 52.8 | 0.4 | 1.9 | 0.0 | 10.7 | 0.0 | 10.7 | 0.75 |
| GP8 | 6.4 | 3.5 | 17.3 | 2.0 | 55.0 | 0.4 | 1.9 | 0.0 | 13.2 | 0.0 | 13.2 | 0.80 |
| GP9 | 7.1 | 3.6 | 16.3 | 2.1 | 54.4 | 0.4 | 1.7 | 0.2 | 13.9 | 0.0 | 13.9 | 0.81 |
| GP10 | 16.0 | 10.4 | 13.7 | 1.6 | 44.7 | 1.5 | 0.0 | 0.0 | 12.1 | 0.0 | 12.1 | 0.81 |
| GP11 | 7.3 | 3.6 | 18.0 | 1.8 | 54.2 | 0.0 | 0.0 | 0.0 | 15.1 | 0.0 | 15.1 | 0.79 |
| GP12 | 7.1 | 3.7 | 16.8 | 2.1 | 54.0 | 0.0 | 2.9 | 0.0 | 13.4 | 0.0 | 13.4 | 0.81 |
| GP13 | 6.7 | 3.4 | 16.7 | 1.9 | 58.5 | 0.0 | 3.1 | 0.0 | 9.5 | 0.0 | 9.5 | 0.81 |
| GP14 | 7.0 | 3.4 | 15.3 | 1.9 | 54.7 | 0.4 | 1.8 | 0.0 | 15.0 | 0.0 | 15.0 | 0.82 |
| GP15 | 7.0 | 3.4 | 16.9 | 1.9 | 52.7 | 0.0 | 2.8 | 0.0 | 15.4 | 0.0 | 15.4 | 0.81 |
| GP16 | 7.4 | 3.5 | 16.2 | 2.2 | 53.5 | 0.4 | 1.9 | 0.0 | 14.2 | 0.0 | 14.2 | 0.81 |
| GP17 | 6.9 | 3.4 | 18.3 | 1.8 | 50.5 | 0.0 | 2.7 | 0.0 | 16.2 | 0.0 | 16.2 | 0.79 |
| GP19 | 6.9 | 2.9 | 13.4 | 1.9 | 56.6 | 0.0 | 3.0 | 0.0 | 15.0 | 0.0 | 15.0 | 0.85 |
| GP20 | 7.0 | 3.2 | 16.3 | 2.1 | 58.2 | 0.0 | 3.2 | 0.2 | 9.5 | 0.0 | 9.5 | 0.81 |

TABLE 8
Seed oil composition of Arabidopsis Ven9 or BU18 lines and transgenic derivatives carrying the BpPDAT gene.

| | | | | | | | | | | | Total | |
|---|---|---|---|---|---|---|---|---|---|---|---|---|
| Plant | C16:0 | C18:0 | C18:1 | C18:1n7 | C18:2 | C20:0 | C18:3 | C20:1 | Ver | C18:2E | Epoxy | ODP |
| Ven9 | 5.5 | 3.4 | 35.3 | 0.0 | 8.7 | 0.0 | 21.6 | 16.5 | 5.2 | 2.3 | 7.6 | 0.52 |
| Ven9 | 6.7 | 4.0 | 38.3 | 0.0 | 6.6 | 0.0 | 18.8 | 16.6 | 4.5 | 2.2 | 6.7 | 0.46 |
| Ven9 | 5.1 | 3.1 | 33.2 | 0.0 | 9.5 | 0.0 | 22.3 | 18.2 | 5.6 | 2.2 | 7.8 | 0.54 |
| GL1 | 5.4 | 3.7 | 26.1 | 0.0 | 12.4 | 0.0 | 29.3 | 18.1 | 5.0 | 0.0 | 5.0 | 0.64 |
| GL3 | 5.0 | 3.8 | 27.6 | 0.0 | 11.4 | 0.0 | 27.8 | 16.9 | 4.3 | 1.8 | 6.1 | 0.62 |
| GL4 | 4.9 | 3.5 | 20.4 | 0.0 | 14.6 | 0.0 | 31.1 | 18.2 | 4.1 | 1.5 | 5.6 | 0.72 |
| GL5 | 4.9 | 3.2 | 25.7 | 0.0 | 13.0 | 0.0 | 24.7 | 19.0 | 5.8 | 2.0 | 7.8 | 0.64 |
| GL7 | 5.2 | 3.5 | 26.8 | 0.0 | 11.1 | 0.0 | 24.6 | 19.9 | 5.1 | 2.0 | 7.1 | 0.62 |
| GL9 | 5.2 | 3.9 | 31.6 | 0.0 | 8.1 | 1.2 | 19.2 | 21.1 | 4.8 | 2.4 | 7.2 | 0.52 |
| GL10 | 5.3 | 3.5 | 27.1 | 0.0 | 10.1 | 0.0 | 28.3 | 17.4 | 5.0 | 2.3 | 7.3 | 0.63 |
| GL11 | 4.5 | 3.2 | 32.0 | 0.0 | 8.9 | 0.0 | 23.1 | 20.1 | 4.5 | 2.1 | 6.6 | 0.55 |
| GL12 | 5.5 | 3.0 | 23.0 | 0.0 | 12.9 | 0.0 | 28.4 | 17.7 | 5.6 | 2.1 | 7.8 | 0.68 |
| GL13 | 5.3 | 3.1 | 26.8 | 0.0 | 11.1 | 0.0 | 27.4 | 18.8 | 4.5 | 2.0 | 6.4 | 0.63 |
| GL14 | 5.4 | 3.6 | 28.3 | 0.0 | 9.6 | 0.0 | 22.2 | 20.6 | 5.8 | 2.6 | 8.4 | 0.59 |
| GL15 | 5.1 | 3.2 | 19.6 | 0.0 | 15.5 | 0.0 | 32.3 | 17.9 | 3.4 | 1.3 | 4.7 | 0.73 |
| GL16 | 5.5 | 3.4 | 25.3 | 0.0 | 11.4 | 0.0 | 25.6 | 20.3 | 4.7 | 2.0 | 6.7 | 0.63 |
| GL17 | 5.1 | 3.2 | 26.5 | 0.0 | 10.1 | 0.0 | 25.7 | 19.4 | 5.7 | 2.4 | 8.1 | 0.62 |
| GL18 | 4.9 | 3.3 | 21.8 | 0.0 | 12.6 | 0.0 | 29.8 | 19.2 | 4.5 | 1.9 | 6.4 | 0.69 |
| GL19 | 5.5 | 4.0 | 34.5 | 0.0 | 0.2 | 0.0 | 23.5 | 22.3 | 5.3 | 2.9 | 8.3 | 0.48 |
| GL20 | 4.8 | 3.3 | 25.6 | 0.0 | 12.9 | 0.0 | 27.7 | 19.5 | 3.5 | 1.2 | 4.7 | 0.64 |
| GL21 | 4.9 | 3.0 | 30.6 | 0.0 | 10.7 | 0.0 | 24.8 | 18.3 | 4.7 | 2.0 | 6.7 | 0.58 |
| GL22 | 5.1 | 3.1 | 33.4 | 0.0 | 9.7 | 0.0 | 21.2 | 19.9 | 5.8 | 2.0 | 7.7 | 0.54 |
| GL24 | 5.2 | 3.3 | 26.7 | 0.0 | 10.2 | 0.0 | 25.5 | 20.4 | 4.8 | 2.3 | 7.0 | 0.62 |
| GL25 | 5.0 | 2.9 | 24.7 | 0.6 | 11.0 | 0.0 | 25.2 | 19.6 | 5.9 | 2.5 | 8.4 | 0.64 |
| GL26 | 5.1 | 3.1 | 27.4 | 0.0 | 12.8 | 0.0 | 23.9 | 18.9 | 5.6 | 1.8 | 7.4 | 0.62 |
| GL28 | 5.3 | 2.9 | 21.8 | 0.0 | 17.1 | 0.0 | 30.5 | 15.8 | 3.9 | 1.1 | 5.0 | 0.71 |
| GL29 | 5.7 | 3.5 | 24.1 | 0.0 | 12.3 | 0.0 | 26.8 | 18.6 | 4.9 | 2.0 | 6.9 | 0.66 |
| GL30 | 5.4 | 3.2 | 30.1 | 0.0 | 10.6 | 0.0 | 22.0 | 20.0 | 5.4 | 2.2 | 7.6 | 0.57 |
| GL31 | 5.1 | 3.4 | 24.6 | 0.0 | 13.2 | 0.0 | 28.2 | 17.8 | 4.9 | 1.8 | 6.7 | 0.66 |
| GL32 | 5.7 | 3.0 | 29.8 | 0.0 | 15.9 | 0.0 | 19.5 | 16.8 | 6.3 | 1.6 | 7.9 | 0.59 |
| GL33 | 5.2 | 3.8 | 27.4 | 0.0 | 10.9 | 0.0 | 26.6 | 18.1 | 4.4 | 1.9 | 6.3 | 0.62 |
| GL35 | 4.9 | 3.6 | 28.0 | 0.0 | 10.9 | 0.0 | 24.2 | 20.5 | 4.9 | 1.8 | 6.7 | 0.60 |
| BU18 | 6.4 | 3.0 | 16.5 | 2.1 | 55.7 | 0.3 | 1.6 | 0.2 | 13.8 | 0.0 | 13.8 | 0.81 |
| BU18 | 6.5 | 3.4 | 17.2 | 1.8 | 54.8 | 0.4 | 1.5 | 0.2 | 13.8 | 0.0 | 13.8 | 0.80 |
| BU18 | 7.3 | 3.4 | 17.1 | 1.7 | 53.7 | 0.4 | 1.5 | 0.0 | 13.9 | 0.0 | 13.9 | 0.80 |
| GO1 | 6.8 | 3.3 | 14.7 | 2.1 | 57.4 | 0.0 | 3.2 | 0.0 | 12.5 | 0.0 | 12.5 | 0.83 |
| GO3 | 6.3 | 3.3 | 14.8 | 1.8 | 53.0 | 0.4 | 2.1 | 0.5 | 17.5 | 0.0 | 17.5 | 0.83 |
| GO4 | 7.0 | 3.5 | 12.8 | 1.9 | 57.5 | 0.0 | 0.0 | 0.0 | 17.3 | 0.0 | 17.3 | 0.85 |
| GO5 | 6.9 | 3.3 | 14.0 | 2.0 | 52.6 | 0.4 | 1.7 | 0.0 | 18.8 | 0.0 | 18.8 | 0.84 |
| GO6 | 6.3 | 2.9 | 15.5 | 1.6 | 56.8 | 0.0 | 2.6 | 0.0 | 14.3 | 0.0 | 14.3 | 0.83 |
| GO7 | 6.8 | 3.4 | 14.5 | 2.0 | 54.6 | 0.4 | 1.8 | 0.0 | 16.2 | 0.0 | 16.2 | 0.83 |
| GO9 | 6.8 | 3.5 | 12.8 | 2.1 | 54.8 | 0.0 | 3.2 | 0.0 | 16.8 | 0.0 | 16.8 | 0.85 |
| GO11 | 6.7 | 3.5 | 15.7 | 2.2 | 51.3 | 0.0 | 4.8 | 1.6 | 13.6 | 0.0 | 13.6 | 0.82 |
| GO12 | 6.2 | 3.0 | 15.4 | 2.2 | 60.3 | 0.0 | 0.0 | 0.0 | 12.9 | 0.0 | 12.9 | 0.83 |
| GO13 | 6.6 | 3.0 | 15.3 | 1.9 | 60.3 | 0.0 | 2.8 | 0.0 | 10.1 | 0.0 | 10.1 | 0.83 |
| GO14 | 6.2 | 3.2 | 12.2 | 2.0 | 57.8 | 0.0 | 2.5 | 0.0 | 15.8 | 0.0 | 15.8 | 0.86 |
| GO15 | 5.8 | 2.8 | 12.5 | 1.9 | 58.6 | 0.0 | 2.5 | 0.1 | 15.4 | 0.0 | 15.4 | 0.86 |
| GO16 | 6.2 | 3.0 | 12.1 | 1.9 | 58.3 | 0.0 | 2.6 | 0.0 | 15.8 | 0.0 | 15.8 | 0.86 |
| GO17 | 6.6 | 3.2 | 12.9 | 2.4 | 58.8 | 0.0 | 2.8 | 0.1 | 12.9 | 0.0 | 12.9 | 0.85 |
| GO18 | 6.5 | 3.3 | 11.0 | 2.3 | 59.1 | 0.0 | 3.0 | 0.0 | 14.5 | 0.0 | 14.5 | 0.87 |
| GO19 | 6.9 | 3.3 | 11.0 | 2.5 | 58.4 | 0.0 | 3.4 | 0.2 | 14.1 | 0.0 | 14.1 | 0.87 |
| GO20 | 6.4 | 3.3 | 13.8 | 1.8 | 57.8 | 0.0 | 2.6 | 0.0 | 14.3 | 0.0 | 14.3 | 0.84 |
| GO22 | 6.2 | 3.0 | 14.4 | 2.0 | 56.8 | 0.0 | 2.5 | 0.0 | 14.8 | 0.0 | 14.8 | 0.84 |
| GO23 | 6.2 | 3.2 | 13.0 | 2.1 | 56.6 | 0.0 | 2.6 | 0.0 | 16.1 | 0.0 | 16.1 | 0.85 |
| GO25 | 6.7 | 3.2 | 11.0 | 2.5 | 57.9 | 0.0 | 3.1 | 0.0 | 15.4 | 0.0 | 15.4 | 0.87 |
| GO26 | 6.5 | 3.7 | 18.3 | 2.0 | 50.6 | 0.4 | 1.8 | 0.0 | 16.6 | 0.0 | 16.6 | 0.79 |
| GO27 | 5.7 | 2.9 | 12.5 | 2.2 | 58.1 | 0.3 | 1.7 | 0.2 | 16.1 | 0.0 | 16.1 | 0.86 |
| GO28 | 6.4 | 3.2 | 13.8 | 2.1 | 59.3 | 0.3 | 1.7 | 0.1 | 12.8 | 0.0 | 12.8 | 0.84 |
| GO29 | 6.2 | 3.3 | 12.9 | 2.1 | 54.6 | 0.3 | 1.7 | 0.1 | 18.5 | 0.0 | 18.5 | 0.85 |
| GO30 | 6.2 | 3.2 | 11.3 | 2.1 | 59.1 | 0.3 | 1.8 | 0.1 | 15.6 | 0.0 | 15.6 | 0.87 |
| GO31 | 6.2 | 3.2 | 13.7 | 2.2 | 58.2 | 0.0 | 2.7 | 0.0 | 13.4 | 0.0 | 13.4 | 0.84 |
| GO32 | 6.5 | 3.2 | 13.7 | 2.3 | 58.5 | 0.3 | 1.9 | 0.2 | 13.4 | 0.0 | 13.4 | 0.84 |
| GO33 | 6.8 | 3.7 | 14.9 | 2.1 | 51.7 | 0.0 | 4.5 | 1.3 | 14.8 | 0.0 | 14.8 | 0.83 |
| GO34 | 6.7 | 3.6 | 15.2 | 1.9 | 50.3 | 0.0 | 4.7 | 1.5 | 15.9 | 0.0 | 15.9 | 0.82 |
| GO35 | 6.9 | 3.6 | 14.4 | 2.1 | 53.1 | 0.0 | 4.0 | 0.8 | 14.8 | 0.0 | 14.8 | 0.83 |
| GO36 | 6.0 | 3.1 | 12.2 | 2.0 | 57.7 | 0.0 | 2.7 | 0.0 | 16.1 | 0.0 | 16.1 | 0.86 |
| GO37 | 6.4 | 3.3 | 14.1 | 1.7 | 59.7 | 0.0 | 2.7 | 0.0 | 11.9 | 0.0 | 11.9 | 0.84 |
| GO38 | 6.4 | 3.3 | 13.4 | 2.1 | 57.9 | 0.3 | 1.7 | 0.1 | 14.5 | 0.0 | 14.5 | 0.85 |
| GO39 | 6.2 | 3.4 | 19.1 | 1.7 | 56.6 | 0.3 | 1.8 | 0.2 | 10.5 | 0.0 | 10.5 | 0.78 |
| GO40 | 6.7 | 3.3 | 14.5 | 2.3 | 56.7 | 0.3 | 2.0 | 0.1 | 13.7 | 0.0 | 13.7 | 0.83 |
| GO41 | 5.9 | 3.0 | 13.6 | 1.9 | 53.5 | 0.0 | 2.5 | 0.0 | 19.4 | 0.0 | 19.4 | 0.85 |
| GO42 | 6.5 | 3.3 | 15.1 | 2.0 | 55.8 | 0.3 | 1.6 | 0.1 | 14.9 | 0.0 | 14.9 | 0.83 |
| GO43 | 6.7 | 3.2 | 11.7 | 2.5 | 59.9 | 0.0 | 3.3 | 0.0 | 12.5 | 0.0 | 12.5 | 0.87 |
| GO44 | 6.5 | 3.3 | 12.7 | 2.2 | 56.9 | 0.0 | 2.8 | 0.0 | 15.3 | 0.0 | 15.3 | 0.86 |
| GO45 | 6.6 | 3.3 | 14.6 | 2.1 | 56.4 | 0.3 | 1.8 | 0.1 | 14.4 | 0.0 | 14.4 | 0.83 |
| GO45 | 6.6 | 3.3 | 14.6 | 2.1 | 56.4 | 0.3 | 1.8 | 0.1 | 14.4 | 0.0 | 14.4 | 0.83 |

### Example 7

### Isolation and Expression of Gene Encoding B. pulchella CDP-Choline Diacylglycerol Choline Phosphotransferase (CPT)

Gene Cloning of A. thaliana AtCPT by PCR

In oilseed lipid synthesis, the major structural lipid of the ER, diacyl-phosphatidylcholine (PC), is also the esterified fatty acid substrate for C18:1 desaturation to C18:2 and C18:3, and for modifying enzymes such as hydroxylases, epoxygenases, acetylenases and conjugases. The acyl-PC is rapidly turned over in developing seeds as an intermediate in TAG synthesis. The enzyme CDP-choline diacylglycerol choline phosphotransferase (CPT) catalyzes the reversible synthesis of PC from DAG, which is one route by which acyl groups are made available for incorporation into TAG via a CoA-independent pathway. CPT genes have been isolated from Arabidopsis thaliana (At3g25585), Saccharomyces cerevisiae (AAA63571), Rattus norvegicus (NP_{—}001007700) and Homo sapiens (NP_{—}001007795) and others.

The full-length protein coding sequence of the A. thaliana gene encoding CDP-choline diacylglycerol choline phosphotransferase, AtCPT (gene At3g25585), was amplified with proof-reading polymerase PfuUltraII (Stratagene) and oligonucleotide primers:

A3-25585-OF
(SEQ ID NO: 90)
5′- GATTCTAGAGAGACCCAATTTGGA-3′
and
A3-25585-OR
(SEQ ID NO: 91)
5′- TTTCCCGGGTCAGGCTTCTTTCCGAGTAATCC-3′

using leaf cDNA as template. The PCR product was cloned as an XbaI-SmaI fragment into pBluescript SK, generating plasmid pXZP037. After sequencing to confirm the gene insert was correct, the EcoRI-SmaI fragment from pXZP037 containing the full-length AtCPT coding sequence was subcloned into the EcoRI-EcoRV sites of pENTR11, resulting in entry plasmid pXZP115E. The gene was then cloned using LR Clonase reactions into yeast expression vector pYES-DEST52 and plant expression vector pXZP391.
Gene Cloning of B. pulchella BpCPT by Library Screening

The XbaI fragment of pXZP115E carrying the full-length AtCPT protein coding sequence was used as a probe to screen the B. pulchella cDNA library at a hybridization temperature of 65° C. The membranes were washed at 65° C. in 2×SSC/0.1% SDS, 1×SSC/0.1% SDS and then in 0.2×SSC/0.1% SDS, each for 10 min. Ten plaques were isolated and used for secondary screening. Four positively hybridizing plaques from the secondary screen were processed by in vivo excision and the nucleotide sequences determined. The full-length sequence of one cDNA, Bp500589, is shown in SEQ ID NO:49. The open reading frame encoding the BpCPT protein started with the ATG start codon at nucleotides 514-516 and was terminated by the TGA stop codon at nucleotides 1681-1683. The deduced amino acid sequence (SEQ ID NO:7) of 389 amino acids shared 78.7% identity and 87.2% similarity with AtCPT.

### Expression of BpCPT

The EcoRI-XhoI fragment of the cDNA clone Bp500589 containing BpCPT was inserted into pENTR11, generated entry plasmid pXZP091E. The gene was then inserted into yeast expression vector pYES-DEST52 and plant expression vector pXZP391, resulted in plasmids pXZP249 and pXZP369, respectively. The CPT function and substrate specificity of the gene expressed in transformed yeast cells is analyzed as described in Example 1. The construct pXZP369 was used to transform the Arabidopsis lines, resulting in transgenic lines.

### Example 8

### Isolation and Expression of Gene Encoding Acyl-CoA:Lysophosphatidylcholine Acyltransferase (LPCAT)

Acyl-CoA:lysophosphatidylcholine acyltransferase (LPCAT; EC 2.3.1.23) catalyzes the acyl-CoA-dependent acylation of lysophosphatidylcholine (LPC) to produce phosphatidylcholine (PC) and CoA. LPCAT activity may affect the incorporation of fatty acid at the sn-2 position of PC where desaturation and/or hydroxylation, epoxygenation, acetylenation or most other modification of the acyl chains occurs. LPCAT belongs to the membrane-bound o-acyltransferase (MBOAT) family of proteins. LPCAT genes have been cloned from mouse (BAE94687, BAF47695), human (BAE94688), rat (BAE94689), yeast (Q06510), and others.

Gene Cloning of A. thaliana LPCAT-Like Sequences

When the A. thaliana genome sequence was examined, two genes (At1g12640 and At1g63050) were considered as candidates to encode membrane bound O-acyl transferase (MBOAT) family proteins, but their specific functions were unknown. The inventors considered these genes as candidates for encoding acyl-CoA:lysophosphatidylcholine acyltransferases (LPCAT). These genes were amplified from Arabidopsis (Columbia) leaf cDNA with proof-reading polymerase PfuUltraII (Stratagene) and primers A1-12640-OF 5′-TCCGAATTCAAAAAAACGGGTTTTCGACACC-3′ (SEQ ID NO:92) and A1-12640-OR 5′-CGTCTCGAGAAGAAGATAACTGCTTATTC-3′ (SEQ ID NO:93) for the first gene, and A1-63050-OF 5′-TTGGAATTCACGCAAGATACAACCATG-3′ (SEQ ID NO:94) and A1-63050-OR 5′-ATCCTCGAGACAACATTATTCTTCTTTTCTGG-3′ (SEQ ID NO:95) for the second.

The resultant amplified fragments were cloned into pGEM-T Easy (Promega) after A-tailed with Taq polymerase, generated plasmids pXZP097TA and pXZP098TA, respectively. After confirming the nucleotide sequences as correct, the genes were inserted as EcoRI-XhoI fragments into pENTR11, resulting in entry plasmids pXZP097E and pXZP098E. From there, the genes were inserted by LR recombinase reactions into yeast expression vector pYES-DEST52 and plant expression vector pXZP391, resulted in plasmids pXZP251, pXZP252, pXZP395 and pXZP396.

Gene Cloning of B. pulchella BpLPCAT-Like Sequences

A BlastX search of the library of B. pulchella EST sequences identified 4 LPCAT-like clones homologous to the two AtLPCAT-like sequences. Among them, clones Bp208211 and Bp208643 had different lengths of 5′-UTR sequence but otherwise were identical and appeared to contain full-length protein coding regions. Bp215446 was a partial cDNA clone that is identical to Bp208211 in the overlapping region. The sequences in these clones were therefore good candidates for encoding LPCAT enzymes and were designated BpLPCAT1. Another clone, Bp211438, also contained a full-length protein coding region that shared homology with the AtLPCAT-like sequences but different to BpLPCAT1, and thus was designated as BpLPCAT2. The complete cDNA sequence of Bp208211 is shown in SEQ ID NO:50.

The open reading frame encoding the BpLPCAT protein started with the ATG start codon at nucleotides 58-60 and was terminated by the TAG stop codon at nucleotides 1435-1437. The deduced amino acid sequence (SEQ ID NO:8) of 459 amino acids shared 74.4% identity and 85.2% similarity to the protein encoded by At1g12640. The complete cDNA sequence of Bp211438 is shown in SEQ ID NO:51. The open reading frame encoding the BpLPCAT-like protein started with the ATG start codon at nucleotides 139-141 and was terminated by the TGA stop codon at nucleotides 1537-1539. The deduced amino acid sequence of 466 amino acids (SEQ ID NO:9) shared 72.9% identity and 83.1% similarity to the protein encoded by At1g63050. The BpLPCAT and BpLPCAT-like sequences shared 72.9% amino acid identity and 83.1% similarity.

The EcoRI-XhoI fragment of cDNA clone Bp208211 and the BamHI-XhoI fragment of cDNA clone Bp211438 were cloned into pENRT11, resulting in entry plasmids pXZP503E and pXZP504E, respectively. The genes were then cloned by LR recombinase reactions into yeast expression vector pYES-DEST52 and plant expression vector pXZP391, resulted in plasmids pXZP253, pXZP254, pXZP397 and pXZP398.

### Expression of AtLPCAT in Plants

The LPCAT function and substrate specificity of the genes expressed in transformed yeast cells is analyzed as described in Example 1. The constructs pXZ395 and pXZP396 were used to transform the Arabidopsis lines Ven9 and BU18, resulting in transgenic lines co-expressing the genes with the Cpal2 epoxygenase in the seed. Seed oil from T2 seeds obtained from T1 plants is analyzed by GC for fatty acid composition.

### Expression of BpLPCAT in Plants

The LPCAT function and substrate specificity of the genes expressed in transformed yeast cells is analyzed as described in Example 1. The constructs pXZ397 and pXZP398 were used to transform the Arabidopsis lines Ven9 and BU18, resulting in transgenic lines co-expressing the genes with the Cpal2 epoxygenase in the seed.

### Example 9

### Isolation and Expression of Gene Encoding B. pulchella Phospholipase C (BpPLC)

### Gene Cloning of BpPLC

The EST library was screened to identify 9 sequences homologous to an Arabidopsis phospholipase C (PLC) gene (At4g34920) which were assembled into 4 different but closely related sequences. One clone, Bp200315, apparently contained a cDNA (nucleotide sequence SEQ ID NO:52, BpPLC-a) having a full-length protein coding region encoding a protein of 318 amino acids (amino acid sequence SEQ ID NO:10, BpPLC-a) which shared 79.9% identity and 87.1% similarity in amino acid sequence with Arabidopsis PLC (At4g34920). The open reading frame encoding the BpPLC protein started with the ATG start codon at nucleotides 12-14 and was terminated by the TGA stop codon at nucleotides 966-968. Clone Bp214073 was also a full-length cDNA of the BpPLC-a gene. Clones Bp202035, Bp203454 and Bp208755 contained partial-length sequences of BpPLC-a. The gene insert in Bp200315 was cloned as an EcoRI-XhoI fragment into pENTR11, resulting in entry plasmid pXZP100E. The gene was then cloned by LR recombinase reaction into yeast expression vector pYES-DEST52 and plant expression vector pXZP391, resulted in plasmids pXZP250 and pXZP390.

Clone Bp208641 contained a full-length cDNA sequence (SEQ ID NO:53) homologous to A. thaliana phospholipase C (At5g67130, NP_{—}569045). The open reading frame encoding the protein started with the ATG start codon at nucleotides 34-36 and was terminated by the TGA stop codon at nucleotides 1297-1299. Its deduced amino acid sequence (SEQ ID NO:11) shared 65.7% identity and 76.9% similarity to A. thaliana phospholipase C, Accession No. NP_{—}569045. This gene (BpPLC-b) shared only 35.2% nucleotide sequence identity with BpPLC-a and the BpPLC-b protein shared only 12.3% amino acid sequence identity with protein BpPLC-a.

Clone Bp215053 contained a partial-length cDNA sequence of a gene (BpPLC-c, SEQ ID NO:54) homologous to Medicago truncatula phosphoinositide-specific phospholipase C (AAL17948), but having only 46.5% identity to BpPLC-a. The deduced amino acid sequence (SEQ ID NO:12), which was missing about 170 amino acid residues from the N-terminal end, shared 57% identity and 69% similarity to Mt PLC (AAL17948).

Clone Bp205027 contained a partial-length sequence (SEQ ID NO:55) that shared homology to Solanum tuberosum phosphoinositide-specific phospholipase C (CAA63954). The deduced amino acid sequence (SEQ ID NO:13) shared 78.4% identity and 86.5% similarity to A. thaliana phosphoinositide-specific phospholipase C2 (At3g08510, NP_{—}187464) over the sequenced region.

### Expression of BpPLC-a in Plants

The PLC function and substrate specificity of the gene expressed in transformed yeast cells is being analyzed as described in Example 1. The construct pXZP390 was used to transform the Arabidopsis lines Ven9 and BU18, resulting in transgenic lines co-expressing the gene with the Cpal2 epoxygenase in the seed. The transformed seed of a number of lines was harvested and will be analyzed for fatty acid composition.

### Example 10

### Isolation and Expression of B. pulchella Phospholipase D BpPLD)

The phospholipase D (PLD) family of enzymes form a major family of phospholipases that were first discovered and genes encoding them cloned from plants. PLD cleaves phospholipids, producing phosphatidic acid and a free head group such as choline. The enzymes often are differentially regulated by one or more of Ca²⁺, polyphosphoinositides, free fatty acids, G-proteins, N-acylethanolamines, and membrane lipids. The biochemical properties, domain structures, and genome organization of plant PLDs are more diverse than those of other organisms (Qin and Wang, 2002) but yet they can be distinguished from other phospholipases. In Arabidopsis, 12 PLD genes have been identified and are presently grouped into five classes: PLDα (al, At3g15730; α2, At1g52570; α3, At5g25370; α4, At1g55180), PLD/β (β1, At2g42010; β2, At4g00240), PLDγ (γ1, At4g11850; γ2, At4g11830; γ3, At4g11840), PLDδ (At4g35790) and PLDξ At3g16790; ξ2, At3g05630).

### Gene Cloning of BpPLD

Examination of the library of EST sequences identified 48 clones that contained sequences homologous to phospholipase D or other lipases. Seven sequences were homologous to phospholipase D genes which belonged to subfamilies PLDα1 and PLDδ1. Clone Bp213916 contained a full-length protein coding region encoding a protein having homology to PLDα1 and its sequence is shown as SEQ ID NO:56. The open reading frame encoding the protein started with the ATG start codon at nucleotides 125-127 and was terminated by the TAA stop codon at nucleotides 2546-2548. The deduced amino acid sequence of 807 amino acids of the encoded protein is shown as SEQ ID NO:14 and shared 91.0% identity and 94.8% similarity to Ricinus communis (castor bean) phospholipase D alpha 1 precursor (Choline phosphatase 1, Phosphatidylcholine-hydrolyzing phospholipase D 1, Accession No. Q41142). Analysis of this BpPLD protein sequence revealed the existence of N-terminal Ca²⁺/phospholipids-binding C2 domain, two HIND motifs of the PLD family (residues 325-363, -TMFTHHQKIVVVDSAlpsgdperrriVSFVGGIDLCDGR-; and 653-680, FMIYVHTKMMIVDDEYIIIGSANINQRS-). The conserved “IYIENQYF” is also found between two HIED motifs, while the seventh residue, Phe(F), is substituted by a Tyr(Y). PLDα1 prefers to PC substrate than PE substrate. Four clones, Bp200708, Bp202515, Bp204745 and Bp212073 contained partial-length cDNAs, identical to Bp213916 in the overlapping regions and therefore likely to be derived from the same gene. Two other clones, Bp203486 and Bp213575, contained partial length cDNA sequences showing homology to PLDδ1. The BpPLDα1 protein coding region will be inserted into expression plasmids as for the other genes described above.

### Example 11

### Isolation and Expression of Gene Encoding B. pulchella Glycerol-3-phosphate Acyltransferase (BpGPAT)

### Gene Cloning of BpGPAT

By examining the EST library, we identified a partial length cDNA clone Bp203239 that encoded a protein homologous to the A. thaliana glycerol-3-phosphate acyltransferase 4 protein (AtGPAT4). The EcoRI-XhoI fragment from this clone was used as probe to screen the B. pulchella cDNA library at a hybridization temperature of 65° C. The membranes were washed at 65° C. for 10 min each in 2×SSC/0.1% SDS, 0.5×SSC/0.1% SDS and 0.2×SSC/0.1% SDS. Twenty-four plaques were isolated, and seven of them were used for in vivo excision and sequencing. The clone with the longest insert, Bp500619, contained full-length protein coding region whose sequence is shown as (SEQ ID NO:57). The open reading frame encoding the protein started with the ATG start codon at nucleotides 29-31 and was terminated by the TGA stop codon at nucleotides 1532-1534. The deduced amino acid sequence (SEQ ID NO:15) shared 79.1% identity and 87.9% similarity to AtGPAT4 (gene At1g016100), and 80.5% identity and 88.6% similarity to AtGPAT8 (gene At4g00400, later renamed as AtLPAAT). Screening of the B. pulchella cDNA library with the Bp500619 gene insert under lower stringency conditions is underway to isolate other members of GPAT gene family, since there are at least 7 members of the AtGPAT gene family encoding isoforms of GPAT in Arabidopsis (Zheng et al., 2003).

The cDNA insert from clone Bp500619 was cloned as a BamHI-XhoI fragment into pENTR11, generating entry plasmid pXZP505E. The gene was then cloned into yeast expression vector pYES-DEST52 and plant expression vector pXZP391, resulting in pXZP255 and pXZP400.

### Expression of BpGPAT

The GPAT function and substrate specificity of the gene expressed in transformed yeast cells is being analyzed as described in Example 1. The construct pXZP400 was used to transform the Arabidopsis lines Ven9 and BU18, resulting in transgenic lines co-expressing the gene with the Cpal2 epoxygenase in the seed. T2 seeds were harvested from a number of transgenic lines and will be analyzed for fatty acid composition.

### Example 12

### Isolation and Expression of Genes Encoding B. pulchella 1-acyl-glycerol-3-phosphate acyltransferase (BpLPAAT)

### Gene Cloning of BpLPAAT

When the EST library was examined, a partial sequence was identified on clone Bp205065 that encoded a protein which was closely related to Arabidopsis 1-acyl-glycerol-3-phosphate acyltransferase (LPAAT, At4g30580). After the completion of sequencing (SEQ ID NO:58), this clone was shown to encode an acyltransferase-like protein (SEQ ID NO:16) that shared 35.7% identity and 53.6% similarity to Clitoria ternatea putative anthocyanin malonyltransferase (BAF49307) and 35.4% identity and 51.6% similarity to A. thaliana acyltransferase-like protein (AAM65241). The open reading frame encoding the protein started with the ATG start codon at nucleotides 14-16 and was terminated by the TAA stop codon at nucleotides 1391-1393. The EcoRI-XhoI fragment of the insert in Bp205065 was used as a probe to screen the B. pulchella cDNA library at a hybridization temperature of 50° C. The membranes were washed at 50° C. in 2×SSC/0.1% SDS and 1×SSC/0.1% SDS each for 10 min, resulted in 120 positive plaques. Among them, 58 plaques were isolated and used for in vivo excision. Among 11 full-length protein sequences encoded by these gene inserts, all showed at least 90% identity to Bp205065, but all were variant in different amino acid residues. TBlastX search of B. pulchella EST sequences with Bp205065 also identified 5 more clones that shared >90% sequence identity to Bp205065.

The EcoRI-ApaI fragment carrying full-length protein coding region from clone Bp205065 was inserted into pENTR11, resulting in entry plasmid pXZP501E. The gene was then cloned into yeast expression vector pYES-DEST52 and plant expression vector pXZP391, generating plasmids pXZP290 and pXZP601.

Sequences from two further Arabidopsis LPAAT genes (At1g78690, At1g80950) were also used as probes to screen the B. pulchella library. The first of these did not identify positive clones in the library. The probe from At1g80950 was amplified in PCR reactions with forward primer 5′-GGTTAGGTGAAAACAATAATG-3′ (SEQ ID NO:96) and reverse primer 5′-GTCAGGCCAGTAAAATTTCAT-3′ (SEQ ID NO:97) using leaf and flower cDNA as template nucleic acid. The amplification product was cloned into pGEM-T Easy and the expected nucleotide sequence confirmed by sequencing. The NotI-NotI fragment containing the At1g80950 fragment was radio-labelled and used as a probe to screen the Bernardia pulchella cDNA library by hybridization under stringent conditions at 60° C. The membranes were washed twice for 10 min each at 60° C. with 2×SSC/0.1% SDS, followed by two washes for 15 min each at 60° C. with 0.5×SSC/0.1% SDS. Thirteen positive plaques were identified and isolated and used for secondary screening, followed by in vivo excision of plaques that were positive in the secondary screen.

Two nearly identical sequences were obtained, designated Bp500989 (SEQ ID NO:100) and Bp500997 (SEQ ID NO:101). The protein sequence encoded by Bp500989 (SEQ ID NO:98) was 79% identical and 89% similar to the Ricinus communis acyltransferase, Accession No. EEF52537. Bp500997 encoded a very similar protein (SEQ ID NO:99) to that of Bp500989, the differences being that it encoded a slightly longer protein, with the last 13 amino acid residues being different to the last 2 amino acid residues of Bp500989, and having a different 3′-UTR sequence.

The BamHI-XhoI and EcoRI-XhoI fragments carrying full-length protein coding region from clones Bp500989 and Bp500997 were inserted into pENTR11, resulting in entry plasmid pXZP527E and pXZP529E. The genes were then cloned into yeast expression vector pYES-DEST52 and plant expression vector pXZP391, generating plasmids pXZP528, pXZP530 and pXZP628, pXZP630.

### Expression of BpLPAATs

The LPAAT function and substrate specificity of the genes expressed in transformed yeast cells will be analyzed as described in Example 1. These genes in construct pXZP628 and pXZP630 will also be used to transform the Arabidopsis lines Ven9 and BU18 to analyze the effect on vernolic acid accumulation.

### Example 13

### Isolation and Expression of Genes Encoding Other B. pulchella Fatty Acid Metabolic Enzymes

From the library of EST sequences, 4 clones, Bp202974, Bp209013, Bp209314 and Bp213308, were identified that appeared full-length and encoded acyltransferase-like sequences. The full sequences were determined.

The complete sequence of Bp202974 (SEQ ID NO:59) contained a 1646 bp cDNA that encoded a protein which showed homology to A. thaliana putative very long-chain fatty acid condensing enzyme (gene At1g19440) and acyltransferase (gene At4g34510). The open reading frame encoding the protein started with the ATG start codon at nucleotides 99-101 and was terminated by the TAA stop codon at nucleotides 1605-1607. The deduced amino acid sequence (SEQ ID NO:17) shared 84.7% identity and 90.7% similarity to A. thaliana putative very long-chain fatty acid condensing enzyme (NP_{—}173376). The BamHI-ApaI fragment carrying full-length cDNA from clone Bp202974 was cloned into pENTR11, generating entry plasmid pXZP092E. The gene was then cloned into yeast expression vector pYES-DEST52 and plant expression vector pXZP391, generating plasmids pXZP245 and pXZP365.

The complete sequence of the gene insert in Bp209013 (SEQ ID NO:60) contained a 1569 bp DNA that encoded a protein homologous to Gossypium hirsutum acyltransferase-like protein (AAL67994). The open reading frame encoding the protein started with the ATG start codon at nucleotides 71-73 and was terminated by the TAG stop codon at nucleotides 1391-1393. The deduced amino acid sequence (SEQ ID NO:18) shared 74.0% identity and 84.1% similarity to Gossypium hirsutum acyltransferase-like protein (AAL67994), and 63.5% identity and 72.7% similarity to A. thaliana acyltransferase (At5g23940). The BamHI-ApaI fragment carrying the full-length cDNA from clone Bp209013 was cloned into pENTR11, generating entry plasmid pXZP094E. The gene was then cloned into yeast expression vector pYES-DEST52 and plant expression vector pXZP391, generating plasmids pXZP247 and pXZP367.

The complete sequence of Bp209314 (SEQ ID NO:61) contained a 1553 bp cDNA that encoded a protein which was homologous to A. thaliana putative acetyl-CoA acyltransferase (gene At2g33150). The open reading frame encoding the protein started with the ATG start codon at nucleotides 34-36 and was terminated by the TAA stop codon at nucleotides 1417-1419. The deduced amino acid sequence (SEQ ID NO:19) shared 88.8% identity and 93.3% similarity to A. thaliana putative acetyl-CoA acyltransferase (At2g33150), and 86.6% identity and 93.3% similarity to Cucumis sativus acetyl-CoA acyltransferase (CAA47926). Another EST clone, Bp211052, was identical to Bp209314 in an overlapping region and likely represented a cDNA from the same gene. The EcoRI-XhoI fragment carrying the full-length cDNA from clone Bp209314 was cloned into pENTR11, generating entry plasmid pXZP0872E. The gene was then cloned into yeast expression vector pYES-DEST52 and plant expression vector pXZP391, generating plasmids pXZP242 and pXZP385.

The complete sequence of Bp213308 (SEQ ID NO:62) contained a 1870 bp cDNA that encoded a protein that was homologous to A. thaliana putative very long-chain fatty acid condensing enzyme gene At1g04220. The open reading frame encoding the protein started with the ATG start codon at nucleotides 45-47 and was terminated by the TGA stop codon at nucleotides 1569-1571. The deduced amino acid sequence (SEQ ID NO:20) shared 81.2% identity and 86.8% similarity to Gossypium hirsutum beta-ketoacyl-CoA synthase (ABV60087), and 74.1% identity and 84.1 similarity to A. thaliana putative beta-ketoacyl-CoA synthase (NP_{—}171918). The EcoRI-XhoI fragment carrying the full-length cDNA from clone Bp213308 was cloned into pENTR11, generating entry plasmid pXZP088E. The gene was then cloned into yeast expression vector pYES-DEST52 and plant expression vector pXZP391, generating plasmids pXZP243 and pXZP386.

### Example 14

### Isolation and Expression of a Gene Encoding a B. pulchella Epoxygenase

When the EST library was examined, two partial-length clones, Bp202712 (SEQ ID NO:63) and Bp210416, encoded proteins which were homologous to epoxygenase CYP81D2 of the cytochrome P450 type and shared the highest homology to Euphoria lagascae epoxygenase, 33.7% identity and 48.3% similarity in the sequenced region. These two clones were identical except one clone was 4 bases longer at 5′-end, suggesting they were two partial cDNAs from the same gene. The deduced amino acid sequence of partial clone Bp202712 is shown in SEQ ID NO:21. The full-length cDNA clone will be obtained by screening the cDNA library.

Examination of the EST library also identified a FAD2-like sequence encoded by clone Bp203803. The full-length cDNA sequence of Bp203803 was 1492 by long (SEQ ID NO:64). The open reading frame encoding the protein started with the ATG start codon at nucleotides 117-119 and was terminated by the TGA stop codon at nucleotides 1266-1268. The deduced amino acid sequence (SEQ ID NO:22) shared 78.1% identity and 87.0% similarity to A. thaliana FAD2 (At3g12120).

Screening the cDNA library with the EcoRI-EcoRI cDNA fragment from Bp203803 at 50° C. resulted in 60 positive plaques after the membranes were washed at 50° C. in 2×SSC/0.1% SDS, 0.5×SSC/0.1% SDS and 0.2×SSC/0.1% SDS each for 15 min. Thirteen plaques were processed by in vivo excision after purification of single plaques, and their sequences were determined. From these clones, two clones with FAD2-like sequences that were highly homologous but different to Bp203803 were identified. Clone Bp500653 was a partial cDNA clone with a 1122 by cDNA (SEQ ID NO:65). Its deduced amino acid sequence (SEQ ID NO:23) shared 73.1% identity and 81.4% similarity to A. thaliana FAD2 (At3g12120), and 63.5% identity and 70.1% similarity to the protein encoded by Bp203803 (SEQ ID NO: 38). The full-length clone of this sequence will be isolated.

Another clone, Bp500673, contained a full-length cDNA 1433 by in size (SEQ ID NO:66) encoding a FAD2-like protein. The open reading frame encoding the protein started with the ATG start codon at nucleotides 111-113 and was terminated by the TGA stop codon at nucleotides 1260-1262. Its deduced amino acid sequence (SEQ ID NO:24) shared 78.4% identity and 87.2% similarity to A. thaliana FAD2 (At3g12120), and 98.2% identity and 98.7% similarity to Bp203803 (SEQ ID NO:22).

The EcoRI cDNA fragment of FAD-2 like clone Bp203803 was inserted into pENTR11, generated pXZP089E. The EcoRI cDNA fragment of Crepis palaestina Δ12-epoxygenase Cpal2 (Lee et al., 1998) was also cloned into pENTR11, generated pXZP090E. The genes in these plasmids were then cloned into yeast expression vector pYES-DEST52, resulted in plasmids pXZP244 and pXZP286, respectively. The functionality of FAD2-like gene from Bp203803 was being compared to Cpal2 in yeast cells. The addition of the gene from Bp203803 to the yeast cells resulted in production of linoleic acid (C18:2) from oleic acid (C18:1), resulting in 20.3% linoleic acid as a percentage of total fatty acid content, demonstrating that the clone encoded Δ12 desaturase (FAD2). The genes in pXZP089E and pXZP090E were also cloned into plant expression vector pXZP391, and their functions confirmed in transgenic plants. When expressed in Arabidopsis MC49, pXZP089E did not result in production of vernolic acid, showing that this gene did not encode an epoxygenase. Expression plasmids of the gene from clone Bp500673 are being constructed.

### Example 15

### Production of Epoxy Fatty Acid in Linseed

### Expression of Δ12-Epoxygenase Gene Capl2 in Flax

Flax (Linum usitatissimum) sp. Ward was transformed with binary vectors containing the Crepis palaestina Δ12-epoxygenase gene Cpal2 (single gene construct pXZP371) or both Cpal2 and the Crepis palaestina Δ12-desaturase gene Cpdes (double gene construct pXZP373), both expressed under the control of a flax linin gene promoter (WO 01/16340). GC analysis of T₁ seeds showed up to 2.1% epoxy fatty acids from 36 pXZP371 transgenic Tₒ lines and 2.3% epoxy fatty acids from 26 pXZP373 transgenic T₀ lines.

### Expression of Δ12-Epoxygenase Gene Cpal2 in Linola Flax

Linola™ is a flax mutant carrying mutations in both the endogenous Δ15-desaturase fad3 genes leading to high accumulation (70%) of linoleic acid C18:2^{Δ9,12}- the substrate for Δ12-epoxygenase, and low linolenic acid (less than 2%) C18:3^{Δ9,12,15} in the seed oil. Crossing of the transgenic flax plants expressing Cpal2 with plants of the Linola variety was carried out to transfer the Δ12-epoxygenase gene into the Linola background. The crossing generated 3000 F₁ seeds from 67 cross pollinations. F₁ seeds (heterozygotes) from 21 crosses were examined by half seed GC analysis, examining 10 seeds per cross, to identify 6 lines of crossing progeny that contained higher vernolic acid levels in seed oil. F₂ seeds were harvested from these progenies, and planted to harvest F₃ seeds. GC analysis of 10-seed pools from these F₂ plants resulted in up to 11.2% total epoxy fatty acid, with 28.8% of that being C18:3^{Δ9,12,15}, suggested that this F₂ plant (R17xEyre-43-34) was not a homozygote for the fad3 gene mutations. Single seed GC analysis from 10 F₃ seeds of this line identified a seed that contained 15.1% epoxy fatty acids and 2.8% C18:3^{Δ9,12,15}, suggesting that this seed was homozygous for both fad3 gene mutations. F₃ seeds from 4 F₂ plants were chosen based on similar analysis, and planted. GC analysis of F₄ seeds harvested from one F₃ line showed 17.1% total epoxy fatty acids (16.8% vernolic acid and 0.3% epoxy C18:2) with 3.7% C18:3 remaining. This F₃ plant could be the homozygote of both fad3 gene mutations and the Cpal2 transgene. The single seed analysis for this line is underway.

### Example 16

### Expression of Multiple Genes in Combination in Plants

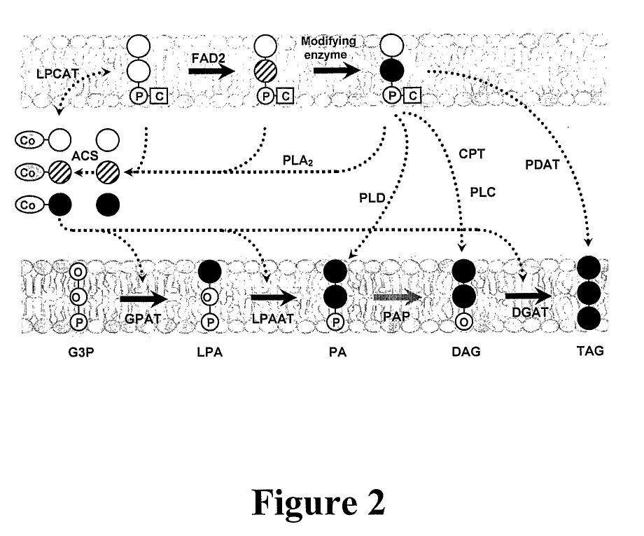

Expression of individual B. pulchella TAG assembly enzymes in the vernolic acid producing Arabidopsis lines is expected to identify the enzymes that have specificity for vernolic acid and thus function in the efficient accumulation of vernolic acid in the transgenic seed. Many enzymes are involved in the TAG assembly as shown in FIG. 2. The function of these enzymes might lead to the increased vernolic acid at different sn positions of TAG. In order to accumulate maximum levels of vernolic acid in seed oil, all 3 sn positions should be occupied by vernolic acid. Therefore, expression of more than one key enzyme, preferably each with specificity for vernolic acid compared to non-epoxygenated fatty acids, from B. pulchella TAG assembly pathway was expected to target all 3 positions and lead to maximum accumulation of vernolic acid in seed oil. Plant expression vector expressing combinations of genes from B. pulchella TAG assembly genes as described above (Examples 2-14) are being constructed and will be expressed in plants for maximum production of vernolic acid.

### Example 17

### Cloning of B. pulchella Other Acyltransferases

B. pulchella EST sequencing generated some partial sequences that shared homology to different acyltransferases. Clones Bp202873 (SEQ ID NO:67) and Bp208395 (SEQ ID NO:68) encoded amino acid sequences (SEQ ID NO:25 and 26 respectively) that were homologous to A. thaliana acyltransferase-like protein (AAM62541).

Clone Bp203237 (SEQ ID NO:69) encoded an amino acid sequence (SEQ ID NO:27) that was homologous to Bp209314.

Clones Bp215205 (SEQ ID NO:70), Bp212247 and Bp204312 represented cDNAs from the same gene, homologous to A. thaliana putative 3-ketoacyl-CoA synthase 4 (KCS-4, Very long-chain fatty acid condensing enzyme 4, NP_{—}173376) (VLCFA condensing enzyme 4) having amino acid sequence identity of 79% over the sequenced region. The partial amino acid sequence encoded by Bp215205 is shown in SEQ ID NO:28.

Clone Bp207528 (SEQ ID NO:71) encoded a partial-length sequence (SEQ ID NO:29) that shared homology with diacylglycerol acyltransferase, but different to BpDGAT1, BpDGAT2 and BpDGAT3. To isolate the full-length cDNA clone corresponding to Bp207528, the cDNA insert of clone Bp207528 was used as probe for screening the Bernardia pulchella cDNA library at high stringency. Among 24 positive plaques, two highly homologous but non-identical sequences were isolated, namely Bp207528a (SEQ ID NO:104) and Bp207528b (SEQ ID NO:105). Bp207528a and Bp207528b differed only at 11 bases in the protein-encoding regions, leading to 1 amino acid residue difference in the encoded proteins. Bp207528b also had a longer 5′-UTR which was relatively GA rich. Bp207528 encodes a protein (Bp207528a provided as SEQ ID NO:102, whereas Bp207528b provided as SEQ ID NO:103) with 325 amino acids which was 69% identical to the Ricinus communis DGAT2 protein sequence, Accession No. AAY16324. When compared to BpDGAT1, DGAT2, DGAT3, the Bp207528 protein was mostly similar to BpDGAT2, both in terms of length of the proteins (327 amino acids in DGAT2) and homology, 68% identity vs less than 12% identity to BpDGAT1 or BpDGAT3. The present inventors have designated this protein as a DGAT-like protein, although it appears to be the first member of a new class of proteins.

EcoRI-XhoI fragments carrying the full-length protein coding regions from both clones were inserted into pENTR11, resulting in entry plasmid pXZP521E and pXZP522E. The genes were then cloned into yeast expression vector pYES-DEST52 and plant expression vector pXZP391, generating plasmids pXZP299, pXZP300 and pXZP621, pXZP622. Function of the proteins will be confirmed in yeast and plant cells.

### Example 18

### Cloning of Genes Encoding Other Lipases from B. pulchella

A total of 56 EST clones were identified as encoding lipase homologues. Besides phospholipases A2, C and D described in Examples 5, 9 and 10, others lipase-like clones are included here.

Clones Bp202796 (full-length cDNA) and Bp210074 (partial length cDNA) contained sequences from same gene, shown as SEQ ID NO:72 and homologous to a Ricinus communis phospholipase (Accession No. AAV66577). The encoded protein (BpPL-a) with amino acid sequence shown as SEQ ID NO:30 had 79.24% identity and 86.3% similarity to the protein having the sequence AAV66577. Clone Bp216215 (SEQ ID NO:73) is a partial sequence same as Bp202796, except there is extra 103 by insertion in the gene, which is potential unprocessed intron. The gene from Bp202796 was cloned as a BamHI-XhoI fragment into pENTR11, resulting in entry plasmid pXZP095E. The gene was then cloned by LR recombinase reaction into yeast expression vector pYES-DEST52 and plant expression vector pXZP391, resulted in plasmids pXZP248 and pXZP368. The construct pXZP368 will be used to transform the Arabidopsis lines Ven9 and BU18, resulting in transgenic lines co-expressing the gene with the Cpal2 epoxygenase in the seed.

Clones Bp201480, Bp215365, Bp212451 contained cDNAs from a gene different to Bp202796 (BpPL-a) but also homologous to Ricinus communis phospholipase AAV66577, with 71.4% identity in the overlapping region with Bp202796. The partial sequence of this gene (assigned as BpPL-b) from full-length cDNA clone Bp201480 is shown in SEQ ID NO:74, and its amino acid sequence is shown in SEQ ID NO:31. The partial sequence from a full-length cDNA clone Bp210076 is same as BpPL-b except 8 bases change when compared to Bp201480. This might be the isomer of BpPL-b.

Clone Bp213710 contains 3′-end partial sequence (SEQ ID NO:75) that encodes an amino acid sequence (SEQ ID NO:32) which shares homology to Ricinus communis phospholipase AAV66577, but is not identical to BpPL-a or BpPL-b. This might be partial sequence of BpPL-b or another gene family member, i.e. BpPL-c.

Clone Bp214230 contained a partial-length sequence (SEQ ID NO:76, BpL-d) that was homologous to Arabidopsis thaliana lipase class 3 family protein (NP_{—}190474, At3g49050). The deduced amino acid sequence is shown in SEQ ID NO:33.

Full-length cDNA clone Bp207119 contained a sequence (BpL-e) that was homologous to another Arabidopsis thaliana lipase class 3 family protein (NP_{—}197365, At5g18640), but divergent to Bp214230. The partial sequence of clone Bp207119 is shown in SEQ ID NO:77, with its deduced amino acid sequence in SEQ ID NO:34.

Clones Bp201211, Bp203733, Bp207631 and Bp214388 were all full-length cDNAs encoding sequences that were identical in the overlapping regions, suggesting they were EST clones derived from the same gene (BpL₇-f) The partial sequence of Bp207631 is shown in SEQ ID NO:78, and the deduced amino acid sequence (SEQ ID NO:35) was homologous to A. thaliana family II extracellular lipase 3 (EXL3, NP_{—}177718, At1g75900) with 59.2% identity or 72.4% similarity.

Clones Bp201783, Bp201784 contained an identical partial-length sequence (SEQ ID NO:79, BpL-g) that was homologous to an Arabidopsis lipase (At1g73920). The deduced amino acid sequence is shown in SEQ ID NO:36.

Clone Bp201910 contained a partial-length sequence (SEQ ID NO:80, BpL-h) that was homologous to Arabidopsis esterase/lipase/thioesterase family protein NP_{—}175685 (At1g52760). The deduced amino acid sequence is shown in SEQ ID NO:37. Bp207135 was a partial cDNA, identical to Bp201910 in the overlapping region.

Bp200659 contained a sequence (SEQ ID NO:81, BpL-i) encoding an amino acid sequence (SEQ ID NO:38) that was homologous to Arabidopsis putative lysophospholipase (AAM60954).

Clone Bp202911 contained a partial-length cDNA sequence (SEQ ID NO:82) coding for an amino acid sequence (SEQ ID NO:39) which is homologous to A. thaliana esterase/lipase/thioesterase family protein (NP174694, At1g34340).

Eighteen clones contained sequences that were homologous to A. thaliana GDSL-motif lipase/hydrolase family proteins. These clones were likely encoded by three members of a gene family. Clone Bp217030 was a full-length cDNA clone that encoded a sequence homologous to A. thaliana GDSL-motif lipase/hydrolase-like protein (AAL48238, At5g45670). The partial nucleotide sequence and deduced amino acid sequence of clone Bp217030 are shown in SEQ ID NO:83 and 40. Clone Bp207002 was the same as Bp217030, but had a shorter 5′-UTR sequence.

Clone Bp204437 was a full-length cDNA with a sequence homologous to another A. thaliana GDSL-motif lipase/hydrolase-like protein (AAM62801, At5g45910), but was different to Bp217030. The partial nucleotide sequence of clone Bp204437 and its deduced amino acid sequence are shown in SEQ ID NO:84 and 41, respectively.

Fifteen other clones were identified having sequences homologous to a third A. thaliana GDSL-motif lipase/hydrolase family protein (NP_{—}974029, At1g54790). Clones Bp207026, Bp208333, Bp212608, Bp215103 and Bp215340 contained full-length cDNA, while Bp212602, Bp201566, Bp207138, Bp202663, Bp203295, Bp215057, Bp209506, Bp203770, Bp217088 and Bp201728 were partial-length cDNA clones, missing different lengths of sequences from the 5′ end. The partial nucleotide sequence of clone Bp215340 and its deduced amino acid sequence are shown in SEQ ID NO:85 and 42, respectively.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 61/125,438 filed 25 Apr. 2008, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotechnology 4:1087.
Almeida and Allshire (2005) TRENDS Cell Biol., 15:251-258.
Banas et al. (2000). Biochem. Soc. Trans. 28:703-705.
Baumlein et al. (1991) Mol. Gen. Genet. 225:459-467.
Baumlein et al. (1992) Plant J. 2:233-239.
Bourque (1995) Plant Sci. 105:125-149.
Broun et al. (1998) Plant J. 13:201-210.
Cahoon et al (2003). Plant J. 34:671-683.
Cahoon et al. (2000) Proc. Natl. Acad. Sci. 96:12935-40.
Capecchi (1980) Cell 22:479-488.
Cheng et at (1996) Plant Cell Rep. 15:653-657.
Clapp (1993) Clin. Perinatol. 20:155-168.
Curiel et al. (1992) Hum. Gen. Ther. 3:147-154.
Dahlqvist et al. (2000) Proc. Natl. Acad. Sci. USA 97:6487-6492.
Dauk et al (2007) Plant Sci. 173:43-49.
Dyer (2002) Plant Physiol. 130:2027-2038.
Dyer and Mullen (2008) Physiologia Plantarum 132: 11-22.
Eglitis et al. (1988) Biotechniques 6:608-614.
Fujimura et al. (1985) Plant Tissue Culture Letters 2:74.
Graham et al. (1973) Virology 54:536-539.
Grant et al. (1995) Plant Cell Rep. 15:254-258.
Harayama (1998). Trends Biotechnol. 16: 76-82.
Haseloff and Gerlach (1988) Nature 334:585-591.
Hatanaka et al (2004) Phytochemistry 65:2189-2196.
Hobbs et al. (2000) Biochem. Soc. Trans. 28:687-689.
Iwabuchi et al (2003) J. Biol. Chem. 278:4603-4610.
Knutzon et al. (1998) J. Biol. Chem. 273:29360-6.
Koziel et al. (1996) Plant. Mol. Biol. 32:393-405.
Lardizabal et al. (2001) J. Biol. Chem. 276:38862-38869.
Lassner et al. (1995) Plant Physiol. 109:1389-1394.
Lee et al. (1998) Science 280:915-918.
Lu et al. (1993) J. Exp. Med. 178:2089-2096.
Lu et al. (2006) Plant J. 45: 847-856.
Millar and Waterhouse (2005) Funct. Integr. Genomics 5:129-135.
Needleman and Wunsch (1970) J. Mol. Biol. 48:443-453.
Pasquinelli et al. (2005) Curr. Opin. Genet. Develop., 15:200-205.
Perriman et al. (1992) Gene 113:157-163.
Qin et al. (2002) Plant Physiol. 128:1057-1068.
Qiu et al. (2001) J. Biol. Chem. 276:31561-31566.
Saha et al. (2006) Plant Physiol. 141:1533-1543.
Schaloske et al. (2000) Biochim Biophys Acta 1761:1246-1259
Senior (1998) Biotech. Genet. Engin. Revs. 15: 79-119.
Shippy et al. (1999) Mol. Biotech. 12:117-129.
Shockey et al. (2006) Plant Cell 18:2294-2313.
Singh et al. (2001) Planta 212: 872-879.
Smith et al. (2000) Nature 407:319-320.
Stalberg et al. (1993) Plant. Mol. Biol. 23:671-683.
Stoutjesdijk et al. (2002) Plant Physiol. 129:1723-1731.
Toriyama et al. (1986) Theor. Appl. Genet. 205:34.
van de Loo et al. (1995) Proc Natl Acad Sci USA. 92:6743-7.
Wagner et al. (1992) Proc. Natl. Acad. Sci. USA 89:6099-6103.
Waterhouse et al. (1998) Proc. Natl. Acad. Sci. USA 95:13959-13964.
Zheng et al. (2003) Plant Cell 15:1872-1887.
Zhou et al. (2006) Funct. Plant Biol. 33: 585-592.
Zou et al. (1999) Plant J. 19:645-653.

1-61. (canceled)

62. A process for producing seedoil, comprising the steps of
i) obtaining a transgenic seed having one or more epoxygenated fatty acids in its seedoil such that at least 23% of the fatty acid content of the seedoil comprises an epoxy group, wherein the seed is from Brassica sp., Gossypium hirsutum, Linum usitatissimum, Helianthus sp., Carthamus tinctorius, Glycine max, Zea mays or Arabidopsis thaliana, and wherein the seedoil has one or more of
a) less than 4% (mol %) of the total fatty acid content of the seedoil is linolenic acid,
b) at least 10% (mol %) of fatty acids esterified at the sn-3 position of total triacylglycerols in the seedoil comprises the epoxy group,
c) at least 10% (mol %) of fatty acids esterified at the sn-2 position of total triacylglycerols in the seedoil comprises the epoxy group,
d) at least 10% (mol %) of fatty acids esterified at the sn-1 position of total triacylglycerols in the seedoil comprises the epoxy group,
e) at least 10% of the seedoil is bi-vernoleate, or
f) at least 4% of the seedoil is tri-vernoleate, and
ii) processing the seed to extract the seedoil.

63. The process of claim 62, wherein the method further comprises harvesting the seed, crushing the seed and/or purifying the seedoil.

64. The process of claim 62, wherein the seedoil has
a) less than 4% (mol %) of the total fatty acid content of the seedoil is linolenic acid,
b) at least 10% (mol %) of fatty acids esterified at the sn-3 position of total triacylglycerols in the seedoil comprises the epoxy group,
c) at least 10% (mol %) of fatty acids esterified at the sn-2 position of total triacylglycerols in the seedoil comprises the epoxy group,
d) at least 10% (mol %) of fatty acids esterified at the sn-1 position of total triacylglycerols in the seedoil comprises the epoxy group, and
e) at least 10% of the seedoil is bi-vernoleate.

65. The process of claim 62, wherein the fatty acids with the epoxy group are C14, C16, C18, C20, C22 or C24 fatty acids or a combination of any two or more thereof.

66. The process of claim 65, wherein the fatty acids with the epoxy group are predominantly C18 fatty acids.

67. The process of claim 66, wherein the fatty acids with the epoxy group are 12,13-epoxy derivatives of C18:1.

68. The process of claim 62, wherein the epoxy group is between carbons 12 and 13 of an acyl chain.

69. The process of claim 62, wherein at least 28% of the fatty acid content of the seedoil comprises an epoxy group.

70. Seedoil comprising one or more epoxygenated fatty acids such that at least 28% (mol %) of the fatty acid content of the seedoil comprises an epoxy group, wherein the seedoil is from Brassica sp., Gossypium hirsutum, Linum usitatissimum, Helianthus sp., Carthamus tinctorius, Glycine max, Zea mays or Arabidopsis thaliana and wherein the seedoil has one or more of
a) less than 4% (mol %) of the total fatty acid content of the seedoil is linolenic acid,
b) at least 10% (mol %) of fatty acids esterified at the sn-3 position of total triacylglycerols in the seedoil comprises the epoxy group,
c) at least 10% (mol %) of fatty acids esterified at the sn-2 position of total triacylglycerols in the seedoil comprises the epoxy group,
d) at least 10% (mol %) of fatty acids esterified at the sn-1 position of total triacylglycerols in the seedoil comprises the epoxy group,
e) at least 10% of the seedoil is bi-vernoleate, or
f) at least 4% of the seedoil is tri-vernoleate.
